(19) United States
(12) Patent Application Publication
(10) Pub. No.: US 2008/0305962 A1
(43) Pub. Date: Dec. 11, 2008

(54) Methods and Kits for the Prediction of Therapeutic Success, Recurrence Free and Overall Survival in Cancer Therapies

(76) Inventor: Ralph Markus Wirtz, Cologne (DE)

Correspondence Address: CHOATE, HALL & STEWART LLP, TWO INTERNATIONAL PLACE, BOSTON, MA, 02110, US

(21) Appl. No.: 11/996,680

(22) PCT Filed: Jul. 20, 2006
(86) PCT No.: PCT/US06/28230

§ 371 Date: Jun. 27, 2008

Related U.S. Application Data
(60) Provisional application No. 60/703,682, filed on Jul. 29, 2005.

(51) Int. Cl. C40B 30/04; C12Q 1/68; C12Q 1/02

(52) U.S. Cl. 506/9; 435/29

(57) ABSTRACT

The invention provides novel compositions, methods and uses, for the prediction, diagnosis, prognosis, prevention and treatment of malignant neoplasia and cancer. The invention further relates to genes that are differentially expressed in tissue of cancer patients versus those of normal “healthy” tissue. Differentially expressed genes for the identification of patients which are likely to respond to chemotherapy are also provided. The present invention relates to methods for prognosis the prediction of therapeutic success in cancer therapy. In a preferred embodiment of the invention it relates to methods for prediction of therapeutic success of combinations of signal transduction inhibitors, therapeutic antibodies, radio- and chemotherapy. The methods of the invention are based on determination of expression levels of 48 human genes which are differentially expressed prior to the onset of anti-cancer chemotherapy. The methods and compositions of the invention are most useful in the investigation of advanced colorectal cancer, but are useful in the investigation of other types of cancer and therapies as well.

### TECHNICAL FIELD OF THE INVENTION

The present invention relates to methods for prognosis the prediction of therapeutic success in cancer therapy. In a preferred embodiment of the invention it relates to methods for prediction of therapeutic success of combinations of signal transduction inhibitors, therapeutic antibodies, radio- and chemotherapy. The methods of the invention axe based on determination of expression levels of 48 human, genes which are differentially expressed prior to the onset of anti-cancer chemotherapy, The methods and compositions of the invention are most useful in the investigation of advanced colorectal cancer, but are useful in the investigation of other types of cancer and therapies as well.

### BACKGROUND OF THE INVENTION AND PRIOR ART

Cancer is the second leading cause of death in the United States after cardiovascular disease. One in three Americans will develop cancer in his or her lifetime, and one of every four Americans will die of cancer. Tumors in general are classified based on different parameters, such as tumor size, invasion status, involvement of lymph nodes, metastasis, histolopathology, immunohistochemical markers, and molecular markers (WHO. International Classification of diseases; Sabin and Wittekind, 1997). With the recent advances in gene chip technology, researchers are increasingly focusing on the categorization of tumors based on the distinct expression of marker genes Sorlie et al., 2001: van't Veer et ah, 2002.

It is a well established fact, that systemic treatment before or after surgery reduces the risk of disease relapse and death in patients with operable cancer. In general, all patients of a given cohort do receive the same treatment, even though many will fail in treatment success. Bio-markers reflecting or being causative for the tumor response can function as sensitive short-term surrogates of long-term outcome. The use of such bio-markers will make chemotherapy more effective for the individual patient and will allow to change regimen early in case of the non responding tumors.

Colorectal cancer (CRC) represents the second leading cause of cancer related deaths in the European Union (Eucan, Cancer Mondial Database 1998). One million people worldwide are diagnosed with this cancer annually, about half of them will succumb, mostly to metastatic disease (Globocan, Cancer Mondial Database 1998). Though much is known about the genetic pathways leading to colorectal neoplasia, the exact molecular mechanisms underlying tumor growth, local invasion, angiogenesis, intravasation and finally metastasis remain poorly understood. Moreover, the relevance of these mechanisms for therapy success or failure have not been resolved and prognostic/predictive markers helping to guide therapy decisions have not yet been identified or validated for clinical routine usage with sufficient level of evidence. Although much effort has been made to develop an optimal clinical treatment course for an individual patient with cancer, only little progress could be achieved predicting the individual's response to a certain therapy.

About 75% percent of patients who are diagnosed with CRC undergo curative treatment. The long term survival of CRC patients depends on the local rumor stage and the potential development of synchronous or metachronous distant metastases. The 5-year-survival rate of CRC patients exceeds 90% in the UICC stage 1 (limited invasion without regional lymph node metastasis), but decreases to below 20% in the UICC stage IV (presence of distant metastasis). Neoadjuvant and adjuvant chemotherapeutic and radiotherapeutic strategies are used to prevent locoregional and distant recurrences, but are effective only in a fraction of stage IV CRC patients. Chemotherapy can lead to a partial remission of distant metastases and can enable secondary palliative surgeries and thereby result in long-term survival. Approximately 25,000 metastatic colorectal cancer patients receive palliative chemotherapy in Germany every year. Response rates of up to 50% have been achieved by the application of modern chemotherapy regimens such as 5-Fluorourical (5-FU), folinic acid (FA) and oxaliplatin. For 15% of the patients, a secondary R0 resection of the liver metastasis is possible and leads to long term survival. Clinical decisions on the therapeutic procedure and extent of resectional treatment in colorectal carcinoma are presently based on imaging and on conventional histopathological features. The diagnostic accuracy of these approaches is limited, which leads to surgical interventions that are most often more radical than required, or to chemotherapeutic treatment of patients who do not benefit from this harsh regimen. As CRC progresses, it can metastasize to the liver and lower a patient's chances of survival. A detailed analysis of reliable prognostic and/or predictive markers for a chemotherapy response would lead to an individually tailored therapy, and would increase the beneficial outcome (e.g. median survival time) and the rate of secondary curative metastatic resection. However, to date, no such predictive markers in the palliative setting have been validated sufficiently. Moreover, biomarkers being indicative of tumor response of metastatic lesions are of special interest also for less advanced stages (i.e. stage I, II and III) as they potentially are also indicative for the response of disseminated and yet dormant cancer cells. However, to date, no such predictive have been analyzed in depth by comparative analysis of primary tumor and corresponding metastasis.

Breast cancer claims the lives of approximately 40,000 women and is diagnosed in approximately 200,000 women annually in the United States alone. For breast cancer, predictions are usually based on standard clinical parameters such as tumor stage and grade, estrogen (ER) and progesterone (PgR) receptors' status, growth rate, overexpression of the HER2/neu and p53 oncogenes. However, evidences about association of ER and/or PgR gene expression with outcome prediction for adjuvant endocrine chemotherapy are still controversial. Studies have shown that levels of ER and PgR gene expression of breast cancer patients are of prognostic importance independently from a subsequent adjuvant chemotherapy. From the theoretical point of view, it is unexpected that the therapeutic response in patients with breast cancer might be independent from the ER/PgR status. It is more probable that the prognostic impact of receptors' expression depends on the impact of other parameters, for example of the ERBB2 receptor. It causes problems finding such factors using conventional biological techniques because all these analyses survey one gene at a time.

Researchers are increasingly focusing on the categorization of tumors based on the distinct expression of marker genes and the DNA microarray technology has been very useful for quantitative measurements of expression levels of thousands of genes simultaneously in one sample. So far this technology has been applied for the classification of cancer tissues e.g., breast tumors [Sorlie et al., 1997], prediction of metastasis and patient's outcome [van't Veer et al., 2002], and tumor response to chemotherapy.

But nevertheless chemotherapy remains a mainstay in therapeutic regimens offered to patients with breast cancer, particularly those who have cancer that has metastasized from its site of origin [Perez, 1999]. There are several chemotherapeutic agents that have demonstrated activity in the treatment of cancer and research is continuously in an attempt to determine optimal drugs and regimens. However, different patients tend to respond differently to the same therapeutic regimen. Currently, the individuals response to certain therapy can only be assessed statistically, based on data of former clinical studies. There are still a great number of patients who will not benefit from a systemic chemotherapy. Most cancers are very heterogeneous in their aggressiveness and treatment response. They contain different genetic mutations and variations affecting growth characteristic and sensitivity to several drugs. Identification of each tumor's molecular fingerprint, then, could help to segregate patients who have particularly aggressive tumors or who need to be treated with specific beneficial therapies. As research involving genetics and associated responses to treatment matures, standard practice will undoubtedly become more individualized, enabling physicians to provide specific treatment regimens matched with a tumors genetic profiles to ensure optimal outcomes. As an alternative therapeutic concept neoadjuvant or primary systemic therapy (PST) can be offered to those patients with either larger inoperable breast cancers. The PST in general do not offer a survival advantage over standard adjuvant treatment, but may identify patients with a pathologically confirmed complete response (CR). In this therapeutic setting such biomarkers capable of predict response can be measured in vivo by correlating gene expression directly to the tumor response.

Assessing the severity and progression of cancerous disease is difficult, and most often entails biopsying. Biopsying involves possible clinical complications and technological difficulties. Moreover, serial sampling to assess early effectiveness of treatment, and elaborate imaging technologies (e.g. computer tomography), clinically are not feasible for routine use. Consequently the development of less invasive and expensive methods, that identify effective regimens before or shortly after first treatment, is of high clinical value.

United States Patent Application Document No. 20030219842 discloses a method of monitoring the progression of disease or cancer treatment effectiveness in a cancer patient by measuring the level of the extracellular domain (BCD) of the epidermal growth factor receptor (EGFR) in a sample taken from the cancer patient, preferably before treatment, at the start of treatment, and at various time intervals during treatment, wherein a decrease in the level of the ECD of the EGFR in the cancer patient compared with the level of the ECD of the EGFR in normal control individuals serves as an indicator of cancer advancement or progression and/or a lack of treatment effectiveness for the patient.

United States Patent Application Document No. 20040151278 discloses a method for detecting the presence of colorectal cancer in an individual, wherein: colorectal cancer is detected by detecting the presence of Reg1α, or TIMP1 nucleic acid or amino acid molecules in a clinical sample obtained from the patient and Reg1α or TIMP1 expression is indicative of the presence of colorectal cancer.

United States Patent Application Document No. 20040146921 discloses a method for providing a patient diagnosis for colon cancer, comprising the steps of; (a) determining the level of expression of one or more genes or gene products in a first biological sample taken from the patient; (b) determining the level of expression of one or more genes or gene products in at least a second biological sample taken from a normal patient sample; and (c) comparing the level of expression of one or more genes or gene products in the first biological sample with the level of expression of one or more genes or gene products in the second biological sample; wherein a change in the level of expression of one or more genes or gene products in the first biological sample compared to the level of expression of one or more genes or gene products in the second biological sample is a diagnostic of the disease.

United States Patent Application Document No. 20040146879 discloses nucleic acid sequences and proteins encoded thereby, as well as probes derived from the nucleic acid sequences, antibodies directed to the encoded proteins, and diagnostic and prognostic methods for detecting and monitoring cancer, especially colon cancer. The sequences disclosed in United States patent Application Document No. 20040146879 have been found to be differentially expressed in samples obtained from colon cancer cell lines and/or colon cancer tissue.

U.S. Pat. No. 6,262,333 discloses nucleic acid sequences and proteins encoded thereby, as well as probes derived from the nucleic acid sequences, antibodies directed to the encoded proteins, and diagnostic methods for detecting cancerous cells, especially colon cancer cells.

Notwithstanding the diagnostic, predicative, and prognostic methods described above, the need continues to exist for improved predictive methods which facilitate an accurate and affordable assessment of whether a patient will respond positively to a particular anti-cancer treatment regimen. Cancer patients cannot afford the time and adverse effects associated with current trial and error therapy selection and inaccurate and risky biopsies.

Reliable predictive markers for a chemotherapy response would lead to an individually tailored therapy, and would increase the beneficial outcome (e.g. median survival time) and the rate of secondary curative metastatic resection. However, to date, no such predictive markers in the palliative setting have been validated sufficiently

### SUMMARY OF THE INVENTION

The present invention is based on the unexpected finding, that 48 human genes are differentially expressed in neoplastic tissue of patients having bad prognosis due to lack of sustained response to anti cancer regimen as compared to patients having better outcome due to sustained response to therapy. Moreover by a knowledge based approach we could identify underlying biological processes that dramatically affect the overall survival of colorectal cancer patients, irrespective of the administered standard therapeutic regimen and which suggest implementation of alternative therapy options. The determination of as few as 4 and up to 48 human genes are sufficient to predict clinical outcome.

It is part of this invention, that the determination of the biological interplay of mechanisms underlying tumor growth, differentiation status, metabolism, loss of adherence and cell-cell contact, local invasion, angiogenesis and intravasation by assessing defined biomarker sets as disclosed within this invention is informative for prognosis and prediction of cancer and can be used to assist therapy decision by analyzing clinical routine specimen. Moreover therapeutic interventions can be deduced targeting these activities in high risk cancer patients and are therefore advantageous for clinical outcome and prolonged survival. Surprisingly, elevated expression of certain EGFR-family members (EGFR) has been found to be prominent in tumors of worse clinical outcome, whereas the simultaneous overexpression of other EGFR family members (e.g. Her-2/neu) did account for less aggressive tumors. Target genes for newly available therapeutics (Iressa, sorafenib, SU 11248, Trastuzumab, Avastin), i.e. EGFR and VEGF alpha were prominently expressed in bad outcome patients, and therefore could be administered to subcohorts of patients. Therefore, especially for the bad prognosis patients, a benefit from such therapeutic strategies could be apparent, as the standard chemotherapy regimen fail in these situations. Similar processes could be identified in breast and colon cancer patients. Therefore this invention comprises also the prediction and prognosis of breast and colon cancer based on said genes as described in table 1.

While not wishing to be bound by any theory, we have discovered that the interplay of certain biological motifs are indicative of cancer progression and can be used to predict the response to anti-cancer regimen. These comprises but is not limited to the following features:

1) differentiation and proliferation status, as determined by HOX and EGFR gene family members
2) recruitment of lymphatic vessels and angiogenesis, as determined by VEGF ligand and VEGFR gene family members
3) metabolism shift to aerobic glycolysis (Warburg effect), as determined by pentose phosphate pathway enzymes and Malic Enzyme gene family members
4) loss of adherence and local invasion, as determined by low PPARG expression, low PLCB4 expression and overexpression of MMP gene family members
5) proliferative and anti-proliferative signaling activities, as determined by expression of MAP3K5, Conductin, PLCB4, as well as expression of HOX, EGFR, TGF ligand and TGF receptor gene family members, as well as mutations in ras/raf, beta-Catenin, APC, EGFR, Her-2/neu, TGFβR2 and SMAD2
6) anti-apoptotic events, as determined by expression of Spondin, PLCB4, BCL-2, p53 as well as mutations in p53, Bax, EGFR and Her-2/neu

Response to an local and systemic therapy may be the prolonged recurrence free survival time after intervention for the primary tumor, but may also reflect the over all survival time. Hence, elevated or decreased levels of expression in one or several of the 48 genes at the time of tumor surgery or prior to any intervention (e.g. biopsy sample) was found to provide valuable information on whether or not a patient is likely to progress despite the given mode of therapy. This would also imply, that those individuals predicted to not progress within a given time frame (e.g. 5 years) will benefit from such chemotherapy regimen and their tumors do respond to the drugs. In a preferred embodiment of the invention, said given mode of chemotherapy is targeted therapy (small molecule inhibitors (e.g., Iressa, Sorafenib, Tarceva, Lapatinib), therapeutic antibodies (e.g. Trastuzumab, Bevacizumab) to the genes being identified as prognostic/predictive markers and chemotherapy.

The present invention relates to 48 human genes, which are differentially expressed in neoplastic tissue of patients responding well to treatment as compared to patients not responding well as determined by overall survival time in the non responding cohort.

The present invention furthermore relates to methods of investigating the response of a patient to anti-cancer chemotherapy by determination of the differential expression of one or several genes of a group of 48 human genes, at the time of tumor excision and before the onset of anti-cancer chemotherapy in a patient. Said investigation of the response can be performed immediately after surgery or at time of first biopsy, at a stage in which other methods can not provide the required information on the patient's response to chemotherapy.

Hence the current invention provides means to decide—shortly after tumor surgery—whether or not a certain mode of chemotherapy is likely to be beneficial to the patient's health and/or whether to maintain or change the applied mode of chemotherapy treatment.

The present invention relates to the identification of 48 human genes being differentially expressed in neoplastic tissue resulting in an altered clinical behavior of a neoplastic lesion. The differential expression of these 48 human genes is not limited to a specific neoplastic lesion in a certain tissue of the human body.

Genes undergoing expressional changes as response to a therapeutic agent, can serve further on as monitoring markers for the therapy and, if they do correlate with the clinical outcome, such genes may also work as efficacy biomarkers.

In preferred embodiments of this invention the neoplastic lesion is colorectal cancer. However this invention also relates to predictive/prognostic value of said genes in lung, ovarian, cervix, stomach, pancreas, head and neck, colon or breast cancer.

The invention relates to various methods, reagents and kits for the prediction of therapeutic success in the therapy of cancer. “Cancer” as used herein includes carcinomas, (e.g., carcinoma in situ, invasive carcinoma, metastatic carcinoma) and pre-malignant conditions, neomorphic changes independent of their histological origin. The compositions, methods, and kits of the present invention comprise comparing the level of mRNA expression of a single or plurality (e.g. 1, 2, 3, 4, 5, 10, 20, 30, 40 or 48) of genes (hereinafter “marker genes”, listed in Table 1, and the respective polypeptide sequences coded by them) in a patient sample, and the average level of expression of the marker gene(s) in a sample from a control subject (e.g., a human subject without cancer). Comparison of the expression level of one or several marker genes can also be performed on any other reference (e.g. tissue samples from responding tumors).

The invention relates further to various compositions, methods, reagents and kits, for prediction of clinically measurable tumor therapy response to a given cancer therapy. The compositions, methods of the present invention comprise comparing the level of mRNA expression of a single or plurality (e.g. 1, 2, 3, 4, 5, 10, 20, 30, 40 or 48) of cancer marker genes in an unclassified patient sample, and the average level of expression of the marker gene(s) in a sample cohort comprising patient responding in different intensity to an administered adjuvant cancer therapy. In preferred embodiments of this invention the specific expression of the marker genes can be utilized for discrimination of responders and non-responders to a targeted or chemotherapeutic intervention.

In further preferred embodiments, the control level of mRNA expression is the average level of expression of the marker gene(s) in samples from several (e.g., 2, 4, 5, 10, 15, 30 or 50) control subjects. These control subjects may also be affected by cancer and be classified by their clinical and not necessarily by their individual expression profile.

As elaborated below, a significant change in the level of expression of one or more of the marker genes (set of marker genes) in the patient sample relative to the control level provides significant information regarding the patient's cancer status and responsiveness to chemotherapy, preferably targeted or chemotherapy. In the compositions, methods, and kits of the present invention the marker genes listed in Table 1 may also be used in combination with well known cancer marker genes (e.g. Ki-67 and PTEN).

According to the invention, the marker gene(s) and marker gene sets are selected such that the positive predictive value of the compositions, methods, and kits of the invention is at least about 10%, preferably about 25%, more preferably about 50% and most preferably about 90% in any of the following conditions: stage 0 cancer patients, stage I cancer patients, stage II cancer patients, stage III cancer patients, stage IV cancer patients, grade I cancer patients, grade II cancer patients, grade III cancer patients, malignant cancer patients, patients with primary carcinomas, and all other types of cancers, malignancies and transformations associated with the lung, ovary, cervix, head and neck, stomach, pancreas, colon or breast.

The detection of marker gene expression is not limited to the detection within a primary, secondary or metastatic lesion of cancer patients, and may also be detected in lymph nodes affected by cancer cells or minimal residual disease cells either locally deposited (e.g. bone marrow, liver, kidney, brain) or freely floating throughout the patients body.

In one embodiment of the compositions, methods, reagents and kits of the present invention, the sample to be analyzed is tissue material from neoplastic lesion taken by aspiration or punctuation, excision or by any other surgical method leading to biopsy or resected cellular material. In one embodiment of the compositions, methods, and kits of the present invention, the sample comprises cells obtained from the patient. The cells may be found in a cell “smear” collected, for example, by a biopsy. In another embodiment, the sample is a body fluid. Such fluids include, for example, blood fluids, lymph, ascitic fluids, gynecological fluids, stool or urine but not limited to these fluids.

In accordance with the compositions, methods, and kits of the present invention the determination of gene expression is not limited to any specific method or to the detection of mRNA. The presence and/or level of expression of the marker gene in a sample can be assessed, for example, by measuring and/or quantifying of:

1) a protein encoded by the marker gene in Table 1 or a protein comprising a polypeptide corresponding to a marker gene in Table 1 or a polypeptide resulting from processing or degradation of the protein (e.g. using a reagent, such as an antibody, an antibody derivative, or an antibody fragment, which binds specifically with the protein or polypeptide)
2) a metabolite which is produced directly (i.e., catalyzed) or indirectly by a protein encoded by the marker gene in Table 1 or by a polypeptide encoded thereby.
3) a RNA transcript (e.g., mRNA, hnRNA) encoded by the marker gene in Table 1, or a fragment of the RNA transcript (e.g. by contacting a mixture of RNA transcripts obtained from the sample or cDNA prepared from the transcripts with a substrate having nucleic acid comprising a sequence of one or more of the marker genes listed within Table 1 fixed thereto at selected positions). The mRNA expression of these genes can be detected e.g. with DNA-microarray as provided by Affymetrix Inc. or other manufacturers (U.S. Pat. No. 5,556,752). The mRNA expression of these genes can also be detected e.g. with DNA-microarray on basis of planar waveguide technology. In a further embodiment the expression of these genes can be detected with bead based direct fluorescent readout techniques such as provided by Luminex Inc. (WO 97/14028).

The composition, method, and kit of the present invention is particularly useful for identifying patients who will not respond to a certain therapy and therefor have unfavorable clinical outcome. For this purpose the composition, method, and kit comprises comparing

a) the level of expression of a single or plurality of marker genes in a patient sample, wherein at least one (e.g. 2, 5, 10, or 50 or more) of the marker genes is selected from the marker genes of Table 1 and
b) the level of expression of the marker gene in a control subject or any other reference expression pattern. The control subject may either be not affected by cancer or be identified and classified by their clinical response to the particular chemotherapy.

It will be appreciated that in this composition, method, and kit the “therapy” may be any therapy for treating cancer including, but not limited to, chemotherapy, small molecule inhibitor, anti-hormonal therapy, directed antibody therapy, radiation, therapy and surgical removal of tissue, e.g., a tumor. Thus, the compositions, methods, and kits of the invention may be used to evaluate a patient before, during and after therapy, for example, to evaluate the reduction in tumor burden.

In another aspect, the invention provides a composition, method, and kit for in vitro selection of a therapy regime (e.g. the kind of chemotherapeutic argents) for inhibiting cancer in a patient. This composition, method, and kit comprises the steps of:

a) obtaining a sample comprising cancer cells from the patient;
b) separately maintaining aliquots of the sample in the presence of a diverse test compositions;
c) comparing expression of a single or plurality of marker genes, selected from the marker genes listed in Table 1;
in each of the aliquots; and
d) selecting one of the rest compositions which induces a lower level of expression of genes from Table 1 and/or a higher level of expression of genes from Table 1 in the aliquot containing that test composition, relative to the level of expression of each marker gene in the aliquots containing the other test compositions.

The invention further provides a composition, method, and kit of making an isolated hybridoma which produces an antibody useful for assessing whether a patient is afflicted with cancer. The composition, method, and kit comprises isolating a protein encoded by a marker gene listed within Table 3 or a polypeptide fragment of the protein, immunizing a mammal using the isolated protein or polypeptide fragment, isolating splenocytes from the immunized mammal, fusing the isolated splenocytes with an immortalized cell line to form hybridomas, and screening individual hybridomas for production of an antibody which specifically binds with the protein or polypeptide fragment to isolate the hybridoma. The invention also includes an antibody produced by this method. Such antibodies specifically bind to a full-length or partial polypeptide comprising a polypeptide listed in Table 1.

The invention also provides various kits. Such kit comprises reagents for assessing expression of a single or a plurality of genes selected from the marker genes listed in Table 1.

In an additional aspect, the invention provides a kit for assessing the presence of cancer cells. This kit comprises an antibody, wherein the antibody binds specifically with a protein encoded by a marker gene listed within Table 1 or polypeptide fragment of the protein. The kit may also comprise a plurality of antibodies, wherein the plurality binds specifically with the protein encoded by each marker gene of a marker gene set listed in Table 1.

In yet another aspect, the invention, provides a kit for assessing the presence of cancer cells, wherein the kit comprises a nucleic acid probe. The probe hybridizes specifically with a RNA transcript of a marker gene listed within Table 1 or cDNA of the transcript. The kit may also comprise a plurality of probes, wherein each of the probes hybridizes specifically with a RNA transcript of one of the marker genes of a marker gene set listed in Table 1.

It will be appreciated that the compositions, methods, and kits of the present invention may also include additional cancer marker genes including known cancer marker genes. It will further be appreciated that the compositions, methods, and kits may be used to identify cancers other than cancer.

### BRIEF DESCRIPTION OF THE DRAWINGS

FIG. 1: Analysis of candidate genes by 2D Hierarchical clustering based on relative expression of candidate genes as determined by Affymetrix profiling of fresh tissue from primary tumors (PR) and liver metastasis of CRC patients. Response of metastatic lesions as determined by computertomography is depicted as “PR”=Partial response, “SD”=Stable Disease and “PD”=Progressive Disease.Expression levels of adjacent normal tissues (Muc=Mucosa; Liv=liver) are presented. Absolute expression levels normalized by global scaling of each indicated gene are depicted in lines. Patients are depicted in rows, starting with the patient number followed by the tumor type (primary tumor “PR” or metastatic lesion “LM”), Colour code is depicted on the upper left side to visualize tumor response.

FIG. 2A-SIBS analysis of HOX and MMP gene families by 2D Hierarchical clustering based on relative expression of candidate genes as determined by Affymetrix profiling of fresh tissue from primary tumors (PR) and liver metastasis of CRC patients. Response of metastatic lesions as determined by computertomography is depicted as “PR”=Partial response, “SD”=Stable Disease and “PD”=Progressive Disease.Expression levels of adjacent normal tissues (Muc=Mucosa; Liv=liver) are presented. Absolute expression levels normalized by global scaling of each indicated gene are depicted in lines. Patients are depicted in rows, starting with the patient number followed by the tumor type (primary tumor “PR” or metastatic lesion “LM”). Colour code is depicted on the upper left side to visualize tumor response.

FIG. 2B: SIBS analysis of a reduced number of the HOX and MMP gene families (i.e. HOXA9, HOXD11 and MMP7, MMP12) by 2D Hierarchical clustering based on relative expression of candidate genes as determined by Affymetrix profiling of fresh, tissue from primary tumors (PR) and liver metastasis of CRC patients. Response of metastatic lesions as determined by computertomography is depicted as “PR”=Partial response, “SD”=Stable Disease and “PD” Progressive Disease.Expression levels of adjacent normal tissues (Muc=Mucosa; Liv=liver) are presented. Absolute expression levels normalized by global scaling of each indicated gene are depicted in lines. Patients are depicted in rows, starring with the patient number followed by the tumor type (primary tumor “PR” or metastatic lesion “LM”). Colour code is depicted on the upper left side to visualize tumor response.

FIG. 3A: Principal component analysis based on relative expression of HOX and MMP genes as determined by Affymetrix profiling of fresh tissue from primary tumors (PR) and liver metastasis of CRC patients. All HOX and MMP gene family members depicted in table 1 were used for the analysis. Adjacent normal tissues (Muc=Mucosa; Liv=liver are included in the analysis. Response of metastatic lesions as determined by computertomography is depicted as “PR”=Partial response, “SD”=Stable Disease and “PD”=Progressive Disease.

FIG. 3B: Principal component analysis based on relative expression of HOXA9, HOXD11, MMP7 and MMP12 as determined by Affymetrix profiling of fresh tissue from primary tumors (PR) and liver metastasis of CRC patients. Adjacent normal tissues (Muc=Mucosa; Liv=liver are included in the analysis. Response of metastatic lesions as determined by computertomography is depicted as “PR”=Partial response, “SD”=Stable Disease and “PD”=Progressive Disease.

FIG. 4A: SIBS analysis of a reduced number of the HOX and MMP gene families (i.e. HOXA9, HOXD11 and MMP7, MMP12) by 2D Hierarchical clustering based on relative expression of candidate genes as determined by qRT-PCR analysis of fixed tissue from primary tumors of CRC patients. Response of the corresponding metastatic lesions as determined by computertomography is depicted as “PR”=Partial response, “SD”=Stable Disease and “PD” Progressive Disease. Expression levels of adjacent normal tissues (Muc=Mucosa; Liv=liver) are presented. Absolute expression levels after normalization to one housekeeping gene (RPL37A) for each indicated gene are depicted in lines. Patients are depicted in rows and depicted by their patient ID. Colour code is depicted on the upper left side to visualize tumor response.

FIG. 4B: Principal component analysis based on normalized expression of HOXA9, HOXD11, MMP7 and MMP12 as determined by qRT-PCR of fixed tissue from primary tumors of CRC patients. Adjacent normal tissues (Muc=Mucosa; Liv=liver are included in the analysis. Response of the corresponding metastatic lesions to the anti-cancer regimen as determined by computertomography is depicted as “PR” Partial response, “SD”=Stable Disease and “PD” Progressive Disease.

FIG. 5A: SIBS analysis of a reduced number of the HOX and MMP gene families (i.e. HOXA9, HOXD11 and MMP7, MMP12) by 2D Hierarchical clustering based on relative expression of candidate genes as determined by qRT-PCR analysis of fixed tissue from primary tumors of CRC patients. Overall survival of the patients suffering the respective primary tumors is colour coded (“alive> 10 month=green, dead in 7 to 12 month=red, dead<4 month=purple). Absolute expression levels after normalization to one housekeeping gene (RPL37A) for each indicated gene are depicted in lines. Patients are depicted in rows and depicted by their patient ID.

FIG. 5B; Principal component analysis based on normalized expression of HOXA9, HOXD11, MMP7 and MMP12 as determined by qRT-PCR of fixed tissue from primary tumors of CRC patients. Adjacent normal tissues (Muc=Mucosa; Liv=liver are included in the analysis. Overall survival of the patients suffering the respective primary tumors is colour coded (“alive> 10 month=green, dead in 7 to 12 month=red, dead<4 month=purple).

FIG. 6: Principal component analysis based on normalized expression of all HOX genes depicted in table 1 as determined by qRT-PCR of fixed tissue from primary tumors of CRC patients. Adjacent normal tissues (Muc=Mucosa; Liv=liver are included in the analysis. Overall survival of the patients suffering the respective primary tumors is colour coded (“alive> 18 month=light blue, dead in 7 to 12 month=light brown, dead< 4 month=dark brown).

FIG. 7: Relative expression of the ERB receptor tyrosine kinase family members in FFPE tissues from primary tumor resectates of patients as described in Example 1 and as determined by qRT-PCR profiling. Genes are displayed in lines. Survival of patients is depicted above each row, with 1 or 0 meaning “dead” or “alive” and the numbers in brackets meaning month of survival since primary diagnosis.

FIG. 8: illustration of process for model generation and cross-validation. From: Slonim, D. K., Nat. Genet. 2002 December; 32 Suppl:502-8.

FIG. 9: Classification based on K-nearest neighbour analysis based on relative expression of 4 candidate genes (HOXA9, HOXD11, MMP7 and MMP12) as determined by qRT-PCR profiling in primary tumors of mCRC patients and grouping of samples on basis of response of metastatic lesion to 5′FU based anti cancer chemotherapy.

FIG. 10: Classification of patients according to model output.

FIG. 11: Survival time vs. model output (mean of test set). The red line represents the tumour response classification as in FIG. 9

### DETAILED DESCRIPTION OF THE INVENTION

### Definitions

“Differential expression”, or “expression” as used herein, refers to both quantitative as well as qualitative differences in the genes' expression patterns observed in at least two different individuals or samples taken from individuals. Differential expression may depend on differential development, different genetic background of tumor cells and/or reaction to the tissue environment of the tumor. Differentially expressed genes may represent “marker genes,” and/or “target genes”. The expression pattern of a differentially expressed gene disclosed herein may be utilized as part of a prognostic or diagnostic cancer evaluation.

The term “pattern of expression” refers, e.g., to a determined level of gene expression compared either to a reference gene (e.g. housekeeper) or to a computed average expression value (e.g. in DNA-chip analyses). A pattern is not limited to the comparison of two genes but even more related to multiple comparisons of genes to a reference genes or samples. A certain “pattern of expression” may also result and be determined by comparison and measurement of several genes disclosed hereafter and display the relative abundance of these transcripts to each other.

Alternatively, a differentially expressed gene disclosed herein may be used in methods for identifying reagents and compounds and uses of these reagents and compounds for the treatment of cancer as well as methods of treatment. The differential regulation of the gene is not limited to a specific cancer cell type or clone, but rather displays the interplay of cancer cells, muscle cells, stromal cells, connective tissue cells, other epithelial cells, endothelial cells and blood vessels as well as cells of the immune system (e.g. lymphocytes, macrophages, killer cells).

A “reference pattern of expression levels”, within the meaning of the invention shall be understood as being any pattern of expression levels that can be used for the comparison to another pattern of expression levels. In a preferred embodiment of the invention, a reference pattern of expression levels is, e.g., an average pattern of expression levels observed in a group of healthy or diseased individuals, serving as a reference group.

“Primer pairs and probes”, within the meaning of the invention, shall have the ordinary meaning of this term which is well known to the person skilled in the art of molecular biology. In a preferred embodiment of the invention “primer pairs and probes”, shall be understood as being polynucleotide molecules having a sequence identical, complementary, homologous, or homologous to the complement of regions of a target polynucleotide which is to be detected or quantified.

“Individually labeled probes”, within the meaning of the invention, shall be understood as being molecular probes comprising a polynucleotide or oligonucleotide and a label, helpful in the detection or quantification of the probe. Preferred labels are fluorescent labels, luminescent labels, radioactive labels and dyes.

“Arrayed probes”, within the meaning of the invention, shall be understood as being a collection of immobilized probes, preferably in an orderly arrangement. In a preferred embodiment of the invention, the individual “arrayed probes” can be identified by their respective position on the solid support, e.g., on a “chip”.

The phrase “tumor response”, “therapeutic success”, or “response to therapy” refers, in the therapeutic setting to the observation of a defined tumor free, recurrence free or overall survival time (e.g. 2 years, 4 years, 5 years, 10 years). This time period of disease free survival may vary among the different tumor entities but is sufficiently longer than the average time period in which most of the recurrences appear. In a neoadjuvant therapy modality response may be monitored by measurement of tumor shrinkage due to apoptosis and necrosis of the tumor mass.

The term “recurrence” or “recurrent disease” does include distant metastasis that can appear even many years after the initial diagnosis and therapy of a tumor, or to local events such as infiltration of tumor cell into regional lymph nodes, or occurrence of tumor cells at the same site and organ of origin within an appropriate time.

“Prediction of recurrence” or “prediction of success” does refer to the methods an compositions described in this invention. Wherein a tumor specimen is analyzed for it's gene expression and furthermore classified based on correlation of the expression pattern to known ones from reference samples. This classification may either result in the statement that such given tumor will develop recurrence and therefore is considered as a “non responding” tumor to the given therapy, or may result in a classification as a tumor with a prorogued disease free post therapy time.

“Discriminant function analysis” is a technique used to determine which variables discriminate between two or more naturally occurring mutually exclusive groups. The basic idea underlying discriminant function analysis is to determine whether groups differ with regard to a set of predictor variables which may or may not be independent of each other, and then to use those variables to predict group membership (e.g., of new cases).

Discriminant function analysis starts with an outcome variable that is categorical (two or more mutually exclusive levels). The model assumes that these levels can be discriminated by a set of predictor variables which, like ANOVA (analysis of variance), can be continuous or categorical (but are preferably continuous) and, like ANOVA assumes that the underlying discriminant functions are linear. Discriminant analysis does not “partition variation”. It does look for canonical correlations among the set of predictor variables and uses these correlates to build eigenfunctions that explain percentages of the total variation of all predictor variables over all levels of the outcome variable.

The output of the analysis is a set of linear discriminant functions (eigenfunctions) that use combinations of the predictor variables to generate a “discriminant score” regardless of the level of the outcome variable. The percentage of total variation is presented for each function.

In addition, for each eigenfunction, a set of Fisher Discriminant Functions are developed that produce a discriminant score based on combinations of the predictor variables within each level of the outcome variable.

Usually, several variables are included in a study in order to see which variable contribute to the discrimination between groups. In that case, a matrix of total variances and co-variances is generated. Similarly, a matrix of pooled within group variances and co-variances may be generated. A comparison of those two matrices via multivariate F tests is made in order to determine whether or not there are any significant differences (with regard to all variables) between groups. This procedure is identical to multivariate analysis of variance or MANOVA. As in MANOVA, one could first perform the multivariate test, and, if statistically significant, proceed to see which of the variables have significantly different means across the groups.

For a set of observations containing one or more quantitative variables and a classification variable defining groups of observations, the discrimination procedure develops a discriminant criterion to classify each observation into one of the groups. In order to get an idea of how well a discriminant criterion “performs”, it is necessary to classify (a priori) different cases, that is, cases that were not used to estimate the discriminant criterion. Only the classification of new cases enables an assessment of the predictive validity of the discriminant criterion.

In order to validate the derived criterion, the classification can be applied to other data sets. The data set used to derive the discriminant criterion is called the training or calibration data set or patient training cohort. The data set used to validate the performance of the discriminant criteria is called the validation data set or validation cohort.

The discriminant criterion (function(s) or algorithm), determines a measure of generalized squared distance. These distances are based on the pooled co-variance matrix. Either Mahalanobis or Euclidean distance can be used to determine proximity. These distances can be used to identify groupings of the outcome levels and so determine a possible reduction of levels for the variable.

A “pooled co-variance matrix” is a numerical matrix formed by adding together the components of the covariance matrix for each subpopulation in an analysis.

A “predictor” is any variable that may be applied to a function to generate a dependent or response variable or a “predictor value”. In one embodiment of the instant invention, a predictor value may be a discriminant score determined through discriminant function analysis of two or more patient blood markers (e.g., plasma or serum markers). For example, a linear model specifies the (linear) relationship between a dependent (or response) variable T, and a set of predictor variables, the X's, so that

Y=b₀+b₁X₁+b₂+X₂+ . . . +bₖXₖ

In this equation b₀ is the regression coefficient for the intercept and the b₁ values are the regression coefficients (for variables 1 through k) computed from the data.

“Classification trees” are used to predict membership of cases or objects in the classes of a categorical dependent variable from their measurements on one or more predictor variables. Classification tree analysis is one of the main techniques used in so-called Data Mining. The goal of classification trees is to predict or explain responses on a categorical dependent variable, and as such, the available techniques have much in common with the techniques used in the more traditional methods of Discriminant Analysis, Cluster Analysis, Nonparametric Statistics, and Nonlinear Estimation.

The flexibility of classification trees makes them a very attractive analysis option, but this is not to say that their use is recommended to the exclusion of more traditional methods. Indeed, when the typically more stringent theoretical and distributional assumptions of more traditional methods are met, the traditional methods may be preferable. But as an exploratory technique, or as a technique of last resort when traditional methods fail, classification trees are, in the opinion of many researchers, unsurpassed. Classification trees are widely used in applied fields as diverse as medicine (diagnosis), computer science (data structures), botany (classification), and psychology (decision theory). Classification trees readily lend themselves to being displayed graphically, helping to make them easier to interpret than they would be if only a strict numerical interpretation were possible.

“Neural Networks” are analytic techniques modeled after the (hypothesized) processes of learning in the cognitive system and the neurological functions of the brain and capable of predicting new observations (on specific variables) from other observations (on the same or other variables) after executing a process of so-called learning from existing data. Neural Networks is one of the Data Mining techniques. The first step is to design a specific network architecture (that includes a specific number of “layers” each consisting of a certain number of “neurons”). The size and structure of the network needs to match the nature (e.g., the formal complexity) of the investigated phenomenon. Because the latter is obviously not known Very well at this early stage, this task is not easy and often involves multiple “trials and errors.”

The neural network is then subjected to the process of “training.” In that phase, computer memory acts as neurons that apply an iterative process to the number of inputs (variables) to adjust the weights of the network in order to optimally predict the sample data on which the “training” is performed. After the phase of learning from an existing data set, the new network is ready and it can then be used to generate predictions.

In one embodiment of the invention, neural networks can comprise memories of one or more personal or mainframe computers or computerized point of care device.

“Cox Regression Analysis” is a statistical technique whereby Cox proportional-hazards regression is used to analyze the effect of several risk factors on survival. The probability of the endpoint (death, or any other event of interest, e.g. recurrence of disease) is called the hazard. The hazard is modeled as:

H(t)=H_{D}(t)×exp(b₁X₁+b₂X₂+b₂X₃+ . . . +bₖXₖ)

where X₁ . . . Xₖ are a collection of predictor variables and H₀(t) is the baseline hazard at time t, representing the hazard for a person with the value 0 for all the predictor variables.

By dividing both sides of the above equation by H₀(t) and taking logarithms, we obtain:

$$ln ( \frac{H ( t )}{{H}_{0} ( t )} ) = {b}_{1} {X}_{1} + {b}_{2} {X}_{2} + {b}_{3} {X}_{3} + … + {b}_{k} {X}_{k}$$

H(t)/H₀(t) is the hazard ratio. The coefficients bᵢ . . . bₖ are estimated by Cox regression, and can be interpreted in a similar manner to that of multiple logistic regression.

If the covariate (risk factor) is dichotomous and is coded 1 if present and 0 if absent, then the quantity exp(bᵢ) can be interpreted as the instantaneous relative risk of an event, at any time, for an individual with the risk factor present compared with an individual with the risk factor absent, given both individuals are the same on all other covariates. If the covariate is continuous, then the quantity exp(bᵢ) is the instantaneous relative risk of an event, at any time, for an individual with an increase of 1 in the value of the covariate compared with another individual, given both individuals are the same on all other covariates.

“Kaplan Meier curves” are a nonparametric (actuarial) technique for estimating time-related events (the survivorship function). 1 Ordinarily, Kaplan Meier curves are used to analyze death as an outcome. It may be used effectively to analyze time to an endpoint, such as remission. Kaplan Meier curves are a univariate analysis, an appropriate starting technique, and estimate the probability of the proportion of individuals in remission at a particular time, starting from the initiation of active date (time zero), is especially applicable when length of follow-up varies from patient to patient, and takes into account those patients lost during follow-up or not yet in remission at end of a clinical study (e.g., censored patients, where the censoring is non-informative). Kaplan Meier is therefore useful in evaluating remissions following loosing a patient. Since the estimated survival distribution for the cohort study has some degree of uncertainty, 95% confidence intervals may be calculated for each survival probability on the “estimated” curve.

A variety of tests (log-rank, Wilcoxan and Gehen) may be used to compare two or more Kaplan-Meier “curves” under certain well-defined circumstances. Median remission time (the time when 50% of the cohort has reached remission), as well as quantities such as three, five, and ten year probability of remission, can also be generated from the Kaplan-Meier analysis, provided there has been sufficient follow-up of patients.

Kaplan-Meier and Cox regression analysis can be performed by using commercially available software packages, e.g., Graph Pad Prism® and SPSS version 11.

“Receiver Operator Characteristic Curve” (“ROC”): is a graphical representation of the functional relationship between the distribution of a marker's sensitivity and I-specificity values in a cohort of diseased persons and in a cohort of non-diseased persons.

“Area Under the Curve” (“AUC”) is a number which represents the area under a Receiver Operator Characteristic curve. The closer this number is to one, the more the marker values discriminate between diseased and non-diseased cohorts

“McNemar Chi-square Test” (“The McNemar test”) is a statistical test used to determine if two correlated proportions (proportions that share a common numerator but different denominators) are significantly different from each other.

A “nonparametric regression analysis” is a set of statistical techniques that allows the fitting of a line for bivariate data that make little or no assumptions concerning the distribution of each variable or the error in estimation of each variable. Examples are: Theil estimators of location, Passing-Bablok regression, and Deming regression.

“Cut-off values” or “Threshold values” are numerical value of a marker (or set of markers) that defines a specified sensitivity or specificity.

“Biological activity” or “bioactivity” or “activity” or “biological function”, which are used interchangeably, herein mean an effector or antigenic function that is directly or indirectly performed by a polypeptide (whether in its native or denatured conformation), or by any fragment thereof in vivo or in vitro. Biological activities include but are not limited to binding to polypeptides, binding to other proteins or molecules, enzymatic activity, signal transduction, activity as a DNA binding protein, as a transcription regulator, ability to bind damaged DMA, etc. A bioactivity can be modulated by directly affecting the subject polypeptide. Alternatively, a bioactivity can be altered by modulating the level of the polypeptide, such as by modulating expression of the corresponding gene.

The term “marker” or “biomarker” refers a biological molecule, e.g., a nucleic acid, peptide, hormone, etc., whose presence or concentration can be detected and correlated with a known condition, such as a disease state.

The term “marker gene,” as used herein, refers to a differentially expressed gene which expression pattern may be utilized as part of predictive, prognostic or diagnostic process in malignant neoplasia or cancer evaluation, or which, alternatively, may be used in methods for identifying compounds useful for the treatment or prevention of malignant neoplasia and lung, ovarian, cervix, head and neck, stomach, pancreas, colon or breast cancer in particular. A marker gene may also have the characteristics of a target gene.

“Target gene”, as used herein, refers to a differentially expressed gene involved in ovarian, cervix, stomach, pancreas, head and neck, colon or breast cancer in a manner by which modulation of the level of target gene expression or of target gene product activity may act to ameliorate symptoms of malignant neoplasia and lung, ovarian, cervix, head and neck, stomach, pancreas, colon or breast cancer in particular. A target gene may also have the characteristics of a marker gene.

The term “neoplastic lesion” or “neoplastic disease” or “neoplasia” refers to a cancerous tissue this includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The term “cancer” is not limited to any stage, grade, histomorphological feature, invasiveness, agressivity or malignancy of an affected tissue or cell aggregation. In particular stage 0 cancer, stage I cancer, stage II cancer, stage III cancer, stage IV cancer, grade I cancer, grade II cancer, grade III cancer, malignant cancer, primary carcinomas, and all other types of cancers, malignancies and transformations associated with the lung, ovary, cervix, head and neck, stomach, pancreas, colon or breast are included. The terms “neoplastic lesion” or “neoplastic disease” or “neoplasia” or “cancer” are not limited to any tissue or cell type they also include primary, secondary or metastatic lesion of cancer patients, and also comprises lymph nodes affected by cancer cells or minimal residual disease cells either locally deposited (e.g. bone marrow, liver, kidney, brain) or freely floating throughout the patients body.

Furthermore, the term “characterizing the sate of a neoplastic disease” is related to, but not limited to, measurements and assessment of one or more of the following conditions: Type of tumor, histomorphological appearance, dependence on external signal (e.g. hormones, growth factors), invasiveness, motility, state by TNM (2) or similar, agressivity, malignancy, metastatic potential, and responsiveness to a given therapy.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, cell-containing body fluids, free floating nucleic acids, urine, stool, peritoneal fluid, and pleural fluid, or cells therefrom. Biological samples may also include sections of tissues such as frozen or fixed sections taken for histological purposes. A biological sample to be analyzed is tissue material from neoplastic lesion taken by aspiration or punctuation, excision or by any other surgical method leading to biopsy or resected cellular material. Such biological sample may comprises cells obtained from a patient. The cells may be found in a cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “therapy modality”, “therapy mode” “regimen” or “chemo regimen” as well as “therapy regime” refers to a timely sequential or simultaneous administration of anti tumor, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in dose of the single agent, timeframe of application and frequency of administration within a defined therapy window. Currently various combinations of various drugs and/or physical methods, and various schedules are under investigation.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. Arrays wherein the probes axe oligonucleotides are referred to as “oligonucleotide arrays” or “oligonucleotide chips,” A “microarray,” herein also refers to a “biochip” or “biological chip”, an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. A “protein array” refers to an array containing polypeptide probes or protein probes which can be in native form or denatured. An “antibody array” refers to an array containing antibodies which include but are not limited to monoclonal antibodies (e.g. from a mouse), chimeric antibodies, humanized antibodies or phage antibodies and single chain antibodies as well as fragments from antibodies.

The term “agonist”, as used herein, is meant to refer to an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The term “antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide, a ligand or an enzyme substrate. An antagonist can also be a compound that downregulates expression of a gene or which reduces the amount of expressed protein present.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The terms “modulated” or “modulation” or “regulated” or “regulation” and “differentially regulated” as used herein refer to both upregulation (i.e., activation or stimulation (e.g., by agonizing or potentiating) and down regulation [i.e., inhibition or suppression (e.g., by antagonizing, decreasing or inhibiting)].

“Transcriptional regulatory unit” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived. The term “derivative” furthermore refers to phosphorylated forms of a polypeptide sequence or protein.

The term “nucleotide analog” refers to oligomers or polymers being at least in one feature different from naturally occurring nucleotides, oligonucleotides or polynucleotides, but exhibiting functional features of the respective naturally occurring nucleotides (e.g. base paring, hybridization, coding information) and that can be used for said compositions. The nucleotide analogs can consist of non-naturally occurring bases or polymer backbones, examples of which are LNAs, PNAs and Morpholinos. The nucleotide analog has at least one molecule different from its naturally occurring counterpart or equivalent.

The term “equivalent”, with respect to a nucleotide sequence, is understood to include nucleotide sequences encoding functionally equivalent polypeptides. Equivalent nucleotide sequences will include sequences that differ by one or more nucleotide substitutions, additions or deletions, such as allelic variants and therefore include sequences that differ due to the degeneracy of the genetic code. “Equivalent” also is used to refer to amino acid sequences that are functionally equivalent to the amino acid sequence of a mammalian homolog of a marker protein, but which have different amino acid sequences, e.g., at least one, but fewer than 30, 20, 10, 7, 5, or 3 differences, e.g., substitutions, additions, or deletions.

“Homology”, “homologs of,” “homologous”, or “identity” or “similarity” refers to sequence similarity between two polypeptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences.

The term “percent identical” refers to sequence identity between two amino acid sequences or between two nucleotide sequences. Identity can each be determined by comparing a position in each sequence which may be aligned for purposes of comparison. When an equivalent position in die compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for determining sequence identity are well-known and described in the art Preferred nucleic acids used in the instant invention have a sequence at least 70%, and more preferably 80% identical and more preferably 90% and even more preferably at least 95% identical to, or complementary to, a nucleic acid sequence of a mammalian homolog of a gene that expresses a marker as defined previously. Particularly preferred nucleic acids used in the instant invention have a sequence at least 70%, and more preferably 80% identical and more preferably 90% and even more preferably at least 95% identical to, or complementary to a nucleic acid sequence of a mammalian homolog of a gene that expresses a marker as defined previously.

“Prognostic Markers” as used herein refers to factors, that provide information about the clinical outcome of patients with or without treatment. The information provided by prognostic markers is not affected by therapeutic interference.

“Predictive Markers” as used herein refers to factors, that provide information about the possible response of a tumor to a distinct therapeutic agent or regimen

The term “marker” or “biomarker” refers a biological molecule, e.g., a nucleic acid, peptide, hormone, etc., whose presence or concentration can be detected and correlated with a known condition, such as a disease state.

Staging is a method to describe how advanced a cancer is. Staging for colorectal cancer takes into account the depth of invasion into the colon wall, and spread to lymph nodes and other organs. Stage 0 (Carcinoma in Situ): Stage 0 cancer is also called carcinoma in situ. This is a precancerous condition, usually found in a polyp. Stage I (Dukes A): The cancer has spread through the innermost lining of the colon to the second and third layers of the colon wall. It has not spread outside the colon. Stage II (Dukes B): The cancer has spread through the colon wall outside the colon to nearby tissues. Stage III (Dukes C); Cancer has spread to nearby lymph nodes, but not to other parts of the body. Stage IV: Cancer has spread to other parts of the body, e.g. metastasized to the liver or lungs.

“CANCER GENES” or “CANCER GENE” as used herein refers to the polynucleotides Table 1, as well as derivatives, fragments, analogs and homologues thereof, the polypeptides encoded thereby as well as derivatives, fragments, analogs and homologues thereof and the corresponding genomic transcription units which can be derived or identified with standard techniques well known in the art using the information disclosed in Tables 1. The Gene symbol, Gene Description, Reference sequence, Unigene ID, and OMIM number are shown in Table 1.

The term “kit” as used herein refers to any manufacture (e.g. a diagnostic or research product) comprising at least one reagent, e.g. a probe, for specifically detecting the expression of at least one marker gene disclosed in the invention, in particular of those genes listed in Table 1, whereas the manufacture is being sold, distributed, and/or promoted as a unit for performing the methods of the present invention. Also reagents (e.g. immunoassays) to detect the presence, the stability, activity, complexity of the respective marker gene products comprising polypeptides encoded by the genes listed in Table 1 regard as components of the kit. In addition, any combination of nucleic acid and protein detection as disclosed in the invention are regard as a kit.

The present invention provides polynucleotide sequences and proteins encoded thereby, as well as probes derived from the polynucleotide sequences, antibodies directed to the encoded proteins, and predictive, preventive, diagnostic, prognostic and therapeutic uses for individuals which are at risk for or which have malignant neoplasia and lung, ovarian, pancreas, head and neck, stomach, pancreas, colon or breast cancer in particular. The sequences disclosure herein have been found to be differentially expressed in samples from head and neck, colon and breast cancer.

The present invention is based on the identification of 48 genes that are differentially regulated (up- or down regulated) in tumor biopsies of patients with clinical evidence of head and neck, colon and breast cancer. The combined analysis and characterization of the co-expression and interaction of these genes provides newly identified roles for disease outcome. Moreover 4 of these genes are targets of anti-cancer regimen. The detailed analysis of these genes thereby not only provides prognostic information, but also offers possibilities for risk adapted and individualized treatment options.

It is obvious to the person skilled in the art that a reference to a nucleotide sequence is meant to comprise the reference to the associated protein sequence which is coded by said nucleotide sequence.

“% identity” of a first sequence towards a second sequence, within the meaning of the invention, means the % identity which is calculated as follows: First the optimal global alignment between the two sequences is determined with the CLUSTALW algorithm [Thomson J D, Higgins D G, Gibson T J. 1994. ClustalW: Improving the sensitivity of progressive multiple sequence alignment through sequence weighting, positions-specific gap penalties and weight matrix choice. Nucleic Acids Res., 22: 4673-4680], Version 1.8, applying the following command line syntax: ./clustalw -infile=Yinfile.txt -output= -outorder=aligned -pwmatrix=gonnet -pwdnamatrix=clustalw -pwgapopen=10.0-pwgapext=0.1-matrix-gonnet -gapopen=10.0-gapext=0.05-gapdist=8-hgapresidues=GPSNDQERK -maxdiv=40. Implementations of the CLUSTAL W algorithm are readily available at numerous sites on the internet, including, e.g., http://www.ebi.ac.uk.

Thereafter, the number of matches in the alignment is determined by counting the number of identical nucleotides (or amino acid residues) in aligned positions. Finally, the total number of matches is divided by the number of nucleotides (or amino acid residues) of the longer of the two sequences, and multiplied by 100 to yield the % identity of the first sequence towards the second sequence.

The present invention relates to:
1. A method for predicting therapeutic success of a given mode of treatment in a subject having cancer, comprising
   (i) determining the pattern of expression levels of at least 1, 2, 3, 4, 5, 10, 15, 20, 25, 30, 35 or 48 marker genes, comprised in the group of marker genes listed in Table 1,
   (ii) comparing the pattern of expression levels determined in (i) with one or several reference pattern(s) of expression levels,
   (iii) predicting therapeutic success for said given mode of treatment in said subject from the outcome of the comparison in step (ii).
2. A method for adapting therapeutic regimen based on individualized risk assessment for a subject having cancer, comprising
   (i) determining the pattern of expression levels of at least 1, 2, 3, 4, 5, 10, 15, 20, 25, 30, 35 or 48 marker genes, comprised in the group of marker genes listed in Table 1,
   (ii) comparing the pattern of expression levels determined in (i) with one or several reference pattern(s) of expression levels,
   (iii) implementing therapeutic regimen targeting said marker genes in said subject from the outcome of the comparison in step (ii).
3. A method of count 1, wherein said given mode of treatment
   (i) acts on recruitment of lymphatic vessels
   (ii) acts on cell proliferation, and/or
   (iii) acts on cellular differentiation
   (iv) acts on cell motility; and/or
   (v) acts on cell survival, and/or
   (vi) acts on cellular metabolism
   (vii) acts on detoxification
   (viii) comprises administration of a chemotherapeutic agent
4. A method of count 1, 2 or 3, wherein said given mode of treatment comprises chemotherapy (5-FU based, anthracycline based, taxol based), small molecule inhibitors (Iressa, Sorafenib, SU 11248), antibody based regimen (Trastuzumab, avastin), anti-proliferation regimen, pro-apoptotic regimen, pro-differentiation regimen, radiation and surgical therapy.
5. A method of any of counts 1 to 3, wherein a predictive algorithm is used.
6. A method of treatment of a neoplastic disease in a subject, comprising
   (i) predicting therapeutic success for a given mode of treatment in a subject having cancer by the method of any of counts 1 to 4,
   (ii) treating said neoplastic disease in said patient by said mode of treatment, if said mode of treatment is predicted to be successful.
7. A method of selecting a therapy modality for a subject afflicted with a neoplastic disease, comprising
   (i) obtaining a biological sample from said subject,
   (ii) predicting from said sample, by the method of any of counts 1 to 4, therapeutic success in a subject having cancer for a plurality of individual modes of treatment,
   (iii) selecting a mode of treatment which is predicted to be successful in step (ii).
8. A method of any of counts 1 to 6, wherein the expression level is determined
   (i) with a hybridization based method, or
   (ii) with a hybridization based method utilizing arrayed probes, or
   (iii) with a hybridization based method utilizing individually labeled probes, or
   (iv) by real time real time PCR, or
   (v) by assessing the expression of polypeptides, proteins or derivatives thereof, or
   (vi) by assessing the amount of polypeptides, proteins or derivatives thereof.
9. A kit comprising at least 1, 2, 3, 4, 5, 10, 15, 20, 25, 30, 35 or 48 primer pairs and probes suitable for marker genes comprised in the group of marker genes listed in Table 1.
10. A kit comprising at least 1, 2, 3, 4, 5, 10, 15, 20, 25, 30, 35 or 48 individually labeled probes, each having a sequence complementary to any of sequences listed in Table 1.
   11. A kit comprising at least 1, 2, 3, 4, 5, 10, 15, 20, 25, 30, 35 or 48 arrayed probes, each having a sequence complementary to any of the sequences listed in Table 1.

It is apparent to the person, skilled in the art that, in order to determine the expression of a gene, parts and fragments of said gene can be used instead.

The invention also relates to methods for determining the probability of successful application of a given mode of treatment in a subject having lung, ovarian, cervix, head and neck, stomach, pancreas, colon or breast cancer, wherein sequences being homologues to the sequences of Table 1 are used. Preferred homologues have 30, 90, 95, or 99% sequence identity towards the original sequence. Preferably the homologues still have the same biological activity and/or function as have the original molecules.

### Experimental Procedures and Settings

The present invention relates to predicting the successful application of a given mode of treatment to a cancer patient, as those individual will have prolonged disease or overall survival. In a preferred embodiment of the invention, said mode of treatment comprises chemotherapy (5-FU based, anthracycline based), small molecule inhibitors (Iressa, Sorafenib, Tarceva, Lapatinib. SU 31248), antibody based regimen (Trastuzumab, avastin).

Cytotoxic and cytostatic agents are common therapeutics for advanced lung, ovarian, cervix, head and neck, stomach, pancreas, colon or breast cancer. These compounds have been established as important chemotherapeutic agents in the armamentarium of drugs to treat cancer in the 1970s and are still in use. Expression profiles of 20 fresh frozen biopsies of liver metastasis and 11 surgical resectates of synchronous primary colorectal cancer have been obtained by the use of RT-PCR strategies and oligonucleotide microarrays (Affymetrix). 31 tumors, 1 normal liver tissue and 3 normal mucosa tissues were used for marker identification approaches. In addition 49 FFPE tissues from stage IV primary tumor resectates were available for RT-PCR strategies and mutation analysis.

Analyzing the data for 34 fresh frozen tumors by statistical methods as described in EXAMPLES we identified 48 significantly differentially expressed genes listed in Table 1.

Biological Relevance of the Genes which are Part of the Invention

Multiple genes listed in Table 1 are related and represent biological and cellular processes, that are characterized by similar regulation. It is part of this invention, that the combined analysis of such motifs improves the accuracy of the diagnostic analysis with respect to sensitivity, specificity and/or assay robustness. These genes are “siblings”. By the way of illustration but limited to the following examples a few characteristic genes from Tablet are described in greater detail:

### HOX Gene Family

Homeobox genes are regulatory genes encoding nuclear proteins that act as transcription factors, regulating aspects of morphogenesis and cell differentiation during normal embryonic development of several animals. In vertebrates, HOX genes exhibit spatially restricted patterns of expression coincident with the morphogenesis of body-segmented structures. The specific combination of HOX genes expressed in a particular segment determines tissue identity. Vertebrate homeobox genes can be divided in two subfamilies: clustered, or HOX genes, and nonclustered, or divergent, homeobox genes (Nunes et al, 2003). Class I human homeobox-containing genes (HOX genes) are organized in four clusters on different chromosomes. The order of the genes within each cluster is highly conserved throughout evolution suggesting that the physical organization of HOX genes may be (1) essential for their expression and (2) responsible for major biological functions. The homeotic genes, whose products serve as determinants of embryonic cell fate, are expressed in a series of different but partially overlapping domains that extend along the anterior-posterior (A-P) axis of the embryo. The Hox genes share a 180-bp homeobox, which encodes a 60-amino acid homeodomain that binds specifically to DNA. There are 4 Hox gene clusters: HOXA (formerly HOX1) on chromosome 7, HOXB (formerly HOX2) on chromosome 17, HOXC (formerly HOX3) on chromosome 12, and HOXD (formerly HOX4) on chromosome 2. By sequence comparison, the genes of each cluster are assigned to 1 of 13 groups. The order of the HOX genes along the chromosome reflects where they are expressed along the body axis. This principle is followed in homeobox gene nomenclature. For a review of homeobox gene nomenclature (Scott M. P, 1992).

During the last decades, several homeobox genes, clustered and nonclustered ones, were identified in normal tissue, in malignant cells, and in different diseases and metabolic alterations. Homeobox genes are involved in the normal teeth development and in familial teeth agenesis. However, normal development and cancer have a great deal in common, as both processes involve shifts between cell proliferation and differentiation. Many cancers exhibit expression of or alteration in homeobox genes, including leukemias, colon, skin, prostate, breast and ovarian cancers. HOX gene expression has been studied in several human tissues and organs as well as in their neoplastic counterparts (Cillo, 1994). It has been observed (a) characteristic patterns of HOX gene expression for each normal solid organ analyzed, (b) altered HOX gene expression in kidney and colon cancer, (c) a correlation between HOX gene expression and different histological types of primary small cell lung cancer (SCLC) and (d) marked alterations of HOX gene expression among primary and metastatic SCLC variant types. Furthermore, differential patterns of HOX gene expression seem to correlate with the adhesion profile (VLA-2, VLA-5, VLA-6 and ICAM-1) and N-RAS mutation in melanoma. This suggests that HOX genes act as a network of transcriptional regulators involved in the process of cell to cell communication during normal morphogenesis, the alteration of which may contribute to the evolution of cancer. Homeobox genes are a network of genes encoding nuclear proteins functioning as transcriptional regulators. HOX gene expression has been analyzed in normal human colon and in primary and metastatic colorectal carcinomas (de VITA et al, 1993). The majority of HOX genes are active in normal adult colon and their overall expression pattern is characteristic of this organ. Furthermore, the expression of some HOX genes is identical in normal and neoplastic colon indicating that these genes may exert an organ-specific function. In contrast, other HOX genes exhibit altered expression in primary colon cancers and their hepatic metastases which may suggest an association with colon cancer progression. Overall the role of the Hox genes in carcinogenesis is complex and not well understood. In particular there are no data indicating a role of the HOX genes as being of predictive value or of causative importance for the clinical response of tumors to anti-cancer treatment like 5′FU based regimen in colorectal cancer.

By using mice models it could be shown that an overlapping, yet different, set of HOX D genes contribute to the formation of the iliocecal sphincter, which divides the small intestine from the large bowel (Zakany and Duboule, 1999). All homozygous mice with the HOXD1-3 deletions lacked the ileocecal valve, having instead a continuous transition from the lower ileum to the colon. At the ileocecal transition, the smooth muscle layer was thin and disorganized in homozygotes, leading to the absence of the sphincter. Analysis of the upper gut revealed signs of aberrant cell differentiation in the pyloric region of the stomach, where ectopic islands of alkaline phosphatase-positive cells were found in the epithelium. These results indicated that HOXD genes are required to set up physiologic constrictions along the previously unsubdivided gut mesoderm. In the absence of Hoxd function, mice lacked sphincters. Moreover, by performing bidirectional complementation of HOX genes in mice, it has been demonstrated that proteins, which share less than 50% identity in the amino acid sequence, are capable of carrying out equivalent biologic functions in the developmental processes recognized to require respective HOX gene activity. In addition, direct evidence has been provided that the different roles played by these genes during embryogenesis are mainly the result of cis-acting sequences that modulate expression of the individual loci. In contrast, ectopic expression of different HOX genes in defined tumor models induced histologically different tumors (see below), claiming for non interchangeable characteristics of HOX gene factors.

### HOXA9

The HOXA9 gene encodes a class I homeodomam protein potentially involved in myeloid differentiation. HOXA9 gene has been cloned and several splice variants have been identified. Using exon-specific probes in Northern blot analysis, a 1.8-kb homeobox-containing transcript in all fetal tissues tested (brain, lung, liver, and kidney); 2.2- and 3.3-kb transcripts in fetal and adult kidney and in adult skeletal muscle; and a 1.0-kb transcript in all adult and fetal tissues tested has been detected. HOXA9 is phosphorylated by protein kinase C (PKC) and more weakly by casein kinase II. PKC phosphorylates HOXA9 on ser204 and thr205, which are located within a highly conserved N-terminal sequence (STRK), PKC phosphorylation on ser204 decreased Hoxa9 DNA binding affinity in vitro and blocked formation of DNA-binding complexes between endogenous HOXA9 and PBX in a human hematopoietic cell line. Phorbol ester induction of myeloid cell differentiation correlated with phosphorylation of HOXA9 on ser204 and the loss of in vivo DNA binding activity, suggesting that PKC regulates the role of HOXA9 in myeloid cell proliferation and differentiation. HOX genes, which normally regulate mullerian duct differentiation, are not expressed in normal ovarian surface epithelium, but are expressed in epithelial ovarian cancer subtypes according to the pattern of mullerian-like differentiation of the cancers. Ectopic expression of HOXA9 in tumorigenic mouse ovarian surface epithelial cells gave rise to papillary tumors resembling serous ovarian cancers. In contrast, HOXA10 and HOXA11 induced morphogenesis of endometrioid-like and mucinous-like tumors, respectively. HOXA7 showed no lineage specificity, but promoted the abilities of HOXA 9, HOXA 10, and HOXA 11 to induce differentiation along their respective pathways.

### HOXA10

HOXA10 is expressed as 3.0- and 2.2-kb transcripts in a limited number of myeloid cell lines. The HOXA 10 mRNAs are generated by alternative splicing of the 5-prime region to a common 3-prime region containing the homeobox resulting in homeodomains of predicted 496- and 94-amino acids. HOXA10 is expressed in the adult human endometrium. Expression of HOXA 10 dramatically increased during the midsecretory phase of the menstrual cycle, corresponding to the time of implantation and increase in circulating progesterone. Expression of HOXA10 in cultured endometrial cells was stimulated by estrogen or progesterone. Stimulation of HOXA 10 by progesterone was concentration-dependent within the physiologic range, and the effect of estrogen was inhibited by cycloheximide. These results identified sex hormones as novel regulators of HOX gene expression. HOXA 10 may have an important function in regulating endometrial development during the menstrual cycle and in establishing conditions necessary for implantation in the human. HOXA 10 expression has also been demonstrated in the myometrium throughout the menstrual cycle. HOXA 10 expression decreased in the midsecretory phase, coinciding with high serum progesterone levels. Treatment of primary myometrial cell cultures with progesterone decreased HOXA 10 expression in vitro, paralleling the expression seen in vivo. Apparently, differential tissue-specific response of HOXA 10 in response to progesterone is likely mediated by sex steroid receptor coactivators or compressors. HOX genes, which normally regulate mullerian duct differentiation, are not expressed in normal ovarian surface epithelium, but are expressed in epithelial ovarian cancer subtypes according to the pattern of mullerian-like differentiation of the cancers. Ectopic expression of HOXA9 in tumorigenic mouse ovarian Surface epithelial cells gave rise to papillary tumors resembling serous ovarian cancers. In contrast, HOXA 10 and HOXA11 induced morphogenesis of endometrioid-like and mucinous-like tumors, respectively. Hoxa7 showed no lineage specificity, but promoted the abilities of HOXA 9, HOXA 10, and HOXA 11 to induce differentiation along their respective pathways. There are no data on the interplay between sex hormones and HOX gene functions during tumor development.

### HOXD11

HOXD 31 gene is fused to the NUP98 gene in acute myeloid leukemia associated with the translocation t(2;11)(q31;p15). Four genes had been found to be fused to a variety of partner genes in AML: AML1 (RUNX1), MIX, MOZ and TEL (ETV6), in addition to NUP98. Among the partner genes of the NUP98 gene, HOXA9, HOXD13, and PMX1 are homeobox genes and part of their DNA binding homeodomain is fused in-frame to a domain encoding the NH2-terminal FG repeat of the NUP98 gene. In the t(2;11) translocation 2 alternatively spliced 5-prime NUP98 transcripts is fused in-frame to the HOXD11 gene. The NUP98/HOXD fusion genes encode similar fusion proteins, suggesting that NUP98/HOXD11 and NUP98/HOXD13 fusion proteins play a role in leukemogenesis through similar mechanisms. Targeted meiotic recombination has been used to produce unequal recombination between the HOXD13, HOXD12, and HOXD11 loci. Furthermore, some deletions and duplications were engineered along with other mutations in cis. HOXD genes compete for a remote enhancer that recognizes the locus in a polar fashion, with a preference for the 5-prime extremity. Modifications in either the number or topography of HOXD loci induced regulatory reallocations affecting both the number and morphology of digits. These results demonstrated why genes located at the extremity of the cluster are expressed at the distal end of the limbs, following a gradual reduction in transcriptional efficiency, and thus highlight the mechanistic nature of collinearity in limbs. Moreover, RXII, a DNA fragment that displays sequence conservation with the chicken genome and is located between HOXD13 and EVX2, was required along with the HOXD13 locus to implement the position-dependent, preferential activation. Removal of both RXII and the HOXD13 locus abrogated quantitative collinearity. By using an inversion of and a large deficiency in the mouse HoxD cluster, a perturbation in the early collinear expression of HOXD11, HOXD12, and HOXD13 in limb buds led to a loss of asymmetry. Interestingly, ectopic HOX gene expression triggered abnormal Shh transcription, which in turn induced symmetrical expression of HOX genes in digits, thereby generating double posterior limbs. It has been concluded that early posterior restriction of Hox gene products sets up an anterior-posterior prepattern, which determines the localized activation of Shh. This signal is subsequently translated into digit morphologic asymmetry by promoting the late expression of Hoxd genes, 2 collinear processes relying on opposite genomic topographies, upstream and downstream Shh signaling. Interestingly, it has been demonstrated that a wide range of digestive tract tumors, including most of those originating in the esophagus, stomach, biliary tract, and pancreas, but not in the colon, display increased hedgehog pathway activity, which is suppressive by cyclopamine, a hedgehog pathway antagonist. Cyclopamine also suppresses cell growth in vitro and causes durable regression of xenograft tumor in vivo.

### HOXC6

2 distinct forms of HOXC6 were cloned from the human breast cancer cell line MCF7. These cDNAs correspond to 2.2- and 1.8-kb transcripts that differ at their 5-prime ends and encode 153- and 235-amino acid homeodomain-containing proteins, respectively. The 2.2-kb HOXC6 transcript is downregulated in human breast cancer cells, whereas the 1.8-kb transcript is expressed in many human tumors, including breast and ovarian carcinomas. Both HOXC6 gene products can repress transcription from a consensus HOX-binding sequence in MDA-MB231 breast cancer cells and can cooperate with other HOX proteins, such as HOXB7, on their target genes.

### MMP Gene Family

The family of matrix metalloproteinases (MMPs) as the main extracellular matrix remodeling enzymes have been studied extensively. There are at least 24 members of the MMP family that can degrade all constituents of connective tissue and thus facilitate invasion. MMPs can be grouped into collagenases (e.g. MMP1, −8, −13), gelatinases (e.g. MMP2, −9), stromelysins (e.g. MMP3, −10, −11) and matrilysins (e.g. MMP7) according to their substrate specificity. Newer classification systems discriminate 8 classes of MMPs on the basis of common structural motifs (Visse and Nagase, 2003). MMP activity in vivo is tightly controlled by transcriptional activation, by a complex proteolytic activation cascade and by an endogenous system of tissue inhibitors of metalloproteinases (TIMPs). Numerous studies have established increased MMP expression in colorectal cancer tissue compared to normal mucosa, and some have shown direct correlations of MMP levels with tumor stage, grade, invasion, metastasis and prognosis suggesting a pivotal role of these enzymes in the development of a malignant phenotype (Wagenaar-Miller et al, 2004). Furthermore, observational and experimental studies in mice strongly implicate these MMPs in tumor progression as well as metastasis (Shah et al, 1994; Itoh et al, 1998; Masuda and Aoki, 1999; Hasegawa et al, 1998, Matsuyama et al, 2002), and preclinical studies using synthetic MMP inhibitors have revealed marked anti-tumor activity (An et al, 1997; Lozonschi et al, 1999; Aparicio et al, 1999). However, this sharply contrasts with the lack of efficacy of MMP inhibitors in clinical phase III trials where patients with advanced disease were treated (Coussens et al., 2002). It became increasingly clear that the biological role of MMPs is not confined to their ability to degrade extracellular matrix. They also participate in the regulation of cellular processes like differentiation, proliferation, angiogenesis, migration, invasion and apoptosis by interacting with growth factors, cytokines, integrins and cell surface receptors (Leeman et al, 2003), suggesting a complex in vivo function that remains poorly understood.

There is evidence that MMPs are involved in the tumorigenesis of colorectal cancer. MMP activity is the result of interactions between the tumor cells and the microenvironment, i.e. the stroma component. It is therefore likely that the liver microenvironment communicates differently with colorectal cancer cells than the orthotopic microenvironment in the bowel. This idea is supported by cell culture experiments showing different MMP inducibility in fibroblasts from different organs (Fabra et al, 1992). Other groups have found downregulation of various MMPs in metastatic prostate cancer (Dhanasekaran et al., 2001; LaTulippe et ai, 2002). These findings raise the possibility that MMPs do not play an essential role in the biology of metastases. This is in line with the observation that synthetic MMP inhibitors are only effective when given early in the phase of tumor establishment but not once metastatic disease is present (Waagenar-Miller et al, 2004).

Interstitial collagenase (MMP1): MMP1, also called interstitial collagenase, is the main enzyme that cleaves intact fibrillar collagen and has been implicated in tumor invasion and metastasis due to its ability to degrade interstitial stroma (Shiozawa et al, 2000; Bendardaf et al, 2003; Murray et al, 1996). It also has a regulatory role by cleaving other MMPs, namely proMMP2 and −9. MMP1 has been inconsistently upregulated in colorectal cancer primary tumors, but the collectives studied so far included early stage patients with primary resectable disease (Roeb et al, 2004, Sunami et al, 2000).

Matrilysin (MMP7): An important role in colorectal tumorigenesis has been ascribed to MMP7, also called matrilysin. MMP7 possesses strong ECM-degradative activity cleaving proteoglycans, fibronectin, entactin, laminin, gelatin, type TV collagen and insoluble elastin (Wilson and Matrisian, 1996). Other mechanisms of action in the promotion and progression of cancer include its ability to activate the gelatinases MMP2 and −9 (Crabbe et al, 1994; Imai et al, 1995) as well as numerous interactions with growth factor signaling pathways (for review: Leeman et al, 2003). MMP7 is overexpressed in colorectal adenomas and carcinomas (McDonnel et al, 1991; Adachi et al, 2001) and correlates with stage, metastasis and adverse outcome in early invasive and advanced CRC (Masaki et al, 2001; Adachi et al, 1999). Zeng et al. have shown high expression of the active form of MMP7 at the invasive front of colorectal cancer liver metastases suggesting that it participates in the establishment of liver metastases (Zeng et al, 2002).

The stromelysins (MMP3 and MMP11): The role of MMP3 and MMP11 in colorectal carcinogenesis is less clear. Both stromelysins are expressed in the stromal component of CRCs and thought to represent a late event in the progression of these tumors (Newell et al, 1994). MMP3 can activate proMMP7 (Imai et al, 1995) and pro MMP9 (Ramos-DeSimone et al, 1999) and thus contribute to the malignant phenotype. Furthermore, it has been implicated in epithelial-mesenchymal transition by disrupting adherens junctions by cleaving E-Cadherin (Lochter et al, 1997). MMP11^{null} mice exhibit markedly reduced growth of colon cancer cell lines suggesting that it has a role in the microenvironment of a growing tumor (Boulay et al, 2001). Increased MMP3 levels in advanced colorectal cancers has been demonstrated (Roeb et al, 2004).

The gelatinases (MMP2 and MMP9):_ MMP2 and MMP9 possess the ability to degrade basement membranes due to their type IV collagenase activity and have been linked to invasion, angiogenesis and liver metastasis (Zeng et al, 1999; Shah et al, 1994; Matsuyama et al, 2002; Masuda and Aoki, 1999; Liabakk et al, 1996; Parsons et al, 1998; Itoh et al, 1998; Bergers et al, 2000; Zeng et al, 1996). Numerous additional activities, i.e. interactions with growth factors and signaling molecules have been identified making the gelatinases prime candidates for mediating invasion, migration and progression of cancer cells (Giannelli et al., 1997; Leeman et al, 2003). MMP9 has been found to be upregulated in colon cancer but not in rectal cancer compared to normal mucosa (Roeb et al, 2001). Chan et al. analyzed MMP2 expression levels in 65 advanced colorectal cancers using ELISA, Western Blot and in-situ hybridisation and found an increase of MMP2 in the primary tumor but a decrease in the liver metastasis (Chan et al, 2001

Macrophage metalloelastase (MMP12): The role of MMP12 in human tumors is contradictory. On one hand, high tumoral MMP12 expression has been linked to increased elastolytic activity and advanced disease in various human cancers (Balaz et al, 2002), on the other hand antiangiogenic properties have been described for MMP12 due to its ability to convert plasminogen into angiostatin (Dong et al, 1997; Yang et al, 2001).

Besides confirming upregulation of key matrix metalloproteinases in colorectal tumors some MMP expression data establish the concept of MMP downregulation in CRC metastases. However, there are some limitations for such a conclusion. First, the mRNA expression levels cannot differentiate between active and latent forms of MMPs. Some groups have shown selective localization of active MMPs to tumor areas while normal tissue mainly contained inactive forms of the MMP (Zeng and Guillem, 1998), Solely measuring mRNA levels might lead to an underestimation of MMP activity in tumor tissue. Second, MMP activity is the result of delicate tumor-stroma interactions, with some MMPs being made by the tumor cells themselves (e.g. MMP7, MMP13) while others being stroma derived as a response to tumor cell signals (e.g. MMP3), and still others being produced by both tumor and stroma cells (e.g. MMP1). It has been hypothesized that differences in Stroma content account for the Variation of MMP levels in different prognostic groups (Liabakk et al, 1996). If MMPs are mediators of metastasis, one possible consequence of decreased MMP expression in liver metastases could be a reduced ability to metastasize secondarily, e.g. from the liver to the Jung.

Surprisingly, we have found that the expression of MMPs within the primary tumor is of high predictive and prognostic value even for stage IV tumors, which have already metastasized and do exhibit downregulation of multiple MMP expression within the liver metastasis, in particular, if combined with additional biomarkers as disclosed within this invention.

### TIMP Family

The tissue inhibitors of metalloproteinases (TIMPs) are naturally occurring proteins that specifically inhibit matrix metalloproteinases and regulate extracellular matrix turnover and tissue remodeling by forming tight-binding inhibitory complexes with the MMPs. Thus, TIMPs maintain the balance between matrix destruction and formation. An imbalance between MMPs and the associated TIMPs may play a significant role in the invasive phenotype of malignant tumors. The TIMP proteins share several structural features. These include the twelve cysteine residues in conserved regions of the molecule that form six disulfide bonds, essential for the formation of native conformations, and the N-terminal region that is necessary for inhibitory activities. The N-terminus of each TIMP contains a consensus sequence (VIRAK) and each TIMP is translated with a 29 amino acid leader sequence that is cleaved off to produce the mature protein. The C-terminal regions are divergent, which may enhance the selectivity of inhibition and binding efficiency. Although the TEMP proteins share high homology, they may either be secreted extracellularly in soluble form (TIMP-1, TIMP-2 and TIMP-4) or bind to extracellular matrix components (TIMP-3). MMPs and TIMPs can be divided into two groups with respect to gene expression: the majority exhibit inducible expression and a small number are produced constitutively or are expressed at very low levels and are not inducible. Among agents that induce MMP and TIMP production are the inflammatory cytokines TNF alpha and IL1 beta. A marked cell type specificity is a hallmark of both MMP and TIMP gene expression (i.e., a limited number of cell types can be induced to make these proteins).

### TIMP1

TIMP-1 is produced and secreted in soluble form by a variety of cell types and is widely distributed throughout the body. It is an extensively glycosylated protein with a molecular mass of 28.5 kDa. TIMP-1 inhibits the active forms of MMPs, and complexes with the proform of MMP9. Like MMP9, TIMP-1 expression is sensitive to many factors. Increased synthesis of TIMP-1 is caused by a wide variety of reagents that include: TGF beta, EGF, PDGF, FGFb, PMA, alltransretinoic acid (RA), IL1 and IL11. The human TIMP-1 gene, about 0.9 kb, has the chromosomal location of Xp11.23 and encodes a 28,000 MW glycoprotein. TIMP-1 appears to play a major role in modulating the activity of interstitial collagenase as well as a number of connective tissue metalloendoproteases. TIMP-1 functions through the formation of a tight 1:1 complex with active collagenase. Collagenase and related metalloproteases are responsible for much of the remodeling that occurs in connective tissue. The extracellular activity of these enzymes may be regulated by TIMP.

### TIMP2

TIMP-2 (also called CSC-21K) is a 21 kDa glycoprotein that is expressed by a variety of cell types. It forms a non-covalent, stoichiometric complex with both latent and active MMPs. TIMP-2 shows a preference for MMP-2. Addition of purified TIMP2 to activated type TV procollagenase resulted in inhibition of the collagenolytic activity in a stoichiometric fashion. TIMP2 abrogates angiogenic factor-induced endothelial cell proliferation in vitro and angiogenesis in vivo independent of MMP inhibition. These effects required alpha-3/beta-1 integrin-mediated binding of TIMP2 to endothelial cells. Furthermore, TIMP2 induced a decrease in total protein tyrosine phosphatase (PTP) activity associated with beta-1 integrin subunits as well as dissociation of the phosphatase SHP1 from beta-L TIMP2 treatment also resulted in a concomitant increase in PTP activity associated with tyrosine kinase receptors FGFR1 and KDR.

### TIMP3

TIMP-3 was first purified from chicken embryo fibroblasts and identified as ChIMP3. The human homologue of TIMP-3, was originally detected as an inducible serum protein in WI-38 fibroblasts. The TIMP-3 localization differs from that of the other three TIMPs, and is thought to be primarily deposited into the extracellular matrix (ECM). TIMP-3 is insoluble, binds to the ECM associated with a variety of cell types, and is widely distributed throughout the body. TIMP-3 shows 30% amino acid homology with TIMP-1 and 38% homology with TIMP-2. TIMP-3 has been shown to promote the detachment of transformed cells from ECM and to accelerate morphological changes associated with cell transformation. Furthermore, up-regulation of TIMP-3 has been associated with a block in the G1 phase of the cell cycle during differentiation of HL-60 leukemia cells. The human TIMP-3 gene has the chromosomal location of 22q12-22q13. Interestingly, TIMP3 encodes a potent angiogenesis inhibitor and is mutated in Sorsby fundus dystrophy, a macular degenerative disease with submacular choroidal neovascularization. The ability of TIMP3 to inhibit VEGF-mediated angiogenesis has been demonstrated and the potential mechanism by which this occurs has been identified: TIMP3 blocks the binding of VEGF to VEGFR2 and inhibits downstream signaling and angiogenesis. This property seems to be independent of its MMP-inhibitory activity, indicating a new function for TIMP3.

With regard to immune function, the balance of MMP and TIMP determines the net migratory capacity of DCs, while TIMP3 may be a marker for mature DCs. TIMP3 contributes to the tumorigenesis of pancreatic endocrine tumors (PETs). Allelic deletions at chromosome 22q12.3 were detected in about 30 to 60% of PETs, suggesting that inactivation of one or more tumor suppressor genes on this chromosomal arm is important for their pathogenesis. Thirteen of 21 PETs (62%) revealed TIMP3 alterations, including promoter hypermethylation and homozygous deletion. The predominant TIMP3 alteration was promoter hypermethylation, identified in 8 of 18 PETs (44%). It was tumor-specific and corresponded to loss or strong reduction of TIMP3 protein expression. Notably, 11 of 14 PETs (79%) with metastases had TIMP3 alterations, compared with only 1 of 7 PETs (14%) without metastases (P less than 0.02). These data suggested a possibly important role of TIMP3 in the tumorigenesis of human PETs, especially in the development of metastases.

Wildtype TIMP3 is localized entirely to the extracellular matrix (ECM) in both its glycosylated (27 kD) and unglycosylated (24 kD) forms. A COOH-terminally truncated TIMP3 molecule was found to be a non-ECM-bound matrix metalloproteinase (MMP) inhibitor, whereas a chimeric TIMP molecule, consisting of the NH2-terminal domain of TIMP2 fused to the COOH-terminal domain of TIMP3, displayed ECM binding, albeit with a lower affinity than the wildtype TIMP3 molecule. Thus, as in TIMP1 and TIMP2, the NH2-terminal domain is responsible for MMP inhibition, whereas the COOH-terminal domain is most important in mediating the specific functions of the molecule. Deletion of the mouse gene Timp3 resulted in an increase in the activity of TNF-alpha converting enzyme (TACE), constitutive release of TNF, and activation of TNF signaling in the liver. The increase in TNF in Timp3 −/− mice culminated in hepatic lymphocyte infiltration and necrosis, features that are also seen in chronic active hepatitis in humans. This pathology was prevented when deletion of Timp3 was combined with deficiency of tumor necrosis factor receptor superfamily, member 1a (TNFRSF1A). In a liver regeneration model that required TNF signaling, Timp3 −/− mice succumbed to liver failure. Hepatocytes from the null mice completed the cell cycle but then underwent cell death owing to sustained activation of TNF. This hepatocyte cell death was completely rescued by a neutralizing antibody to TNF. Dysregulation of TNF occurred specifically in Timp3 −/− mice and not in mice null for the Timp1 gene. These data indicated that TIMP3 is a crucial innate negative regulator of TNF in both tissue homeostasis and tissue response to injury.

### TIMP4

TIMP-4 was identified by molecular cloning. TIMP-4 shows 37% amino acid identity with TIMP-1 and 51% homology with TIMP-2 and TIMP-3. TIMP-4 is secreted extracellularly, predominantly in heart and brain tissue. It may function in a tissue specific fashion in extracellular matrix (ECM) homeostasis. TIMP-4 has a strong inhibitory effect on the invasion of human breast cancer cells across reconstituted basement membranes suggesting that TIMP-4 may have an important role in inhibiting primary tumor growth and progression. The human TIMP-4 gene has the chromosomal location of 3p25.

### VEGF Ligand and Receptor Families

Vascular endothelial growth factor is a mitogen primarily for vascular endothelial cells. There are various isoforms of VEGFA. The deduced protein has a 26-amino acid signal peptide at its N terminus, and the prominent mature protein contains 165 amino acids. There are VEGF species with 121 amino acids and 189 amino acids, which result from a 44-amino acid deletion at position 116 and a 24-amino acid insertion at position 116, respectively. VEGF shares homology with the PDGF A chain (PDGFA) and B chain (PDGFB), including conservation of all 8 cysteines found in PDGFA and PDGFB. However, VEGF has 8 additional cysteines within its C-terminal 50 amino acids. A VEGF isoform predicted to contain 145 amino acids and to lack exon 7, has been found in tumor cell lines, which has been termed VEGF 145. The VEGF gene contains 8 exons. The various VEGF coding region forms arise through alternative splicing: the 165-amino acid form is missing the residues encoded by exon 6, whereas the 121-amino acid form is missing the residues encoded by axons 6 and 7. VEGFA has been shown to bes mitogenic to adrenal cortex-derived capillary endothelial cells and to several other vascular endothelial cells, but it was not mitogenic toward nonendothelial cells. VEGF, a homodimeric glycoprotein of relative molecular mass 45,000, is the only mitogen that specifically acts on endothelial cells. It may be a major regulator of tumor angiogenesis in vivo. Its expression is upregulated by hypoxia and its cell surface receptor, Flk1, is exclusively expressed in endothelial cells. The importance of VEGF and its receptor system in tumor growth and suggested that intervention in this system provides promising approaches to cancer therapy (Folkman J., 1995).

VEGF A, B and D and placental growth factor constitute a family of regulatory peptides capable of controlling blood vessel formation and permeability by interacting with 2 endothelial tyrosine kinase receptors, FLT1 and KDR/FLK1. Another member of this family VEGFC is the ligand of the related FLT4 receptor involved in lymphatic vessel development. VEGF is a candidate hormone for facilitating glucose passage across the blood-brain barrier under critical conditions. Hypoglycemia is accompanied by a brisk increase in circulating VEGF concentration. VEGF 145 is secreted as an approximately 41-kD homodimer and induces skin induced angiogenesis. VEGF145 inhibited binding by VEGF165 to the KDR/FLK1 receptor in cultured endothelial cells. Like VEGF189, but unlike VEGF 165, VEGF 145 binds efficiently to the extracellular matrix (ECM) by a mechanism that is not dependent on ECM-associated heparan sulfates.

This isoform-specific VEGF receptor (VEGF165R) binds VEGF165 but not VEGF121 and is identical to human neuropilin-1, a receptor for the collapsin/semaphorin family that mediates neuronal cell guidance. When coexpressed in cells with KDR, neuropilin-1 enhances the binding of VEGF165 to KDR and VEGF165-mediated chemotaxis. Conversely, inhibition of VEGF165 binding to neuropilin-1 inhibits its binding to KDR and its mitogenic activity for endothelial cells

VEGF and angiopoietins collaborate during tumor angiogenesis. Angiopoietin-1 is antiapoptotic for cultured endothelial cells and expression of its antagonist angiopoietin-2 was induced in the endothelium of co-opted tumor vessels before their regression. In contrast, marked induction of VEGF expression occurred much later in tumor progression, in the hypoxic periphery of tumor cells surrounding the few remaining internal vessels, as well as adjacent to the robust plexus of vessels at the tumor margin. Expression of Ang2 in the few surviving internal vessels and in the angiogenic vessels at the tumor margin suggested that the destabilizing action of angiopoietin-2 facilitates the angiogenic action of VEGF at the tumor rim

Autocrine endothelial VEGF contributes to the formation of blood vessels in a tumor and promotes its survival. Oxygen gradients can induce a gradient of VEGF expression in the opposite direction. VEGF mediated angiogenic activity in a variety of estrogen target tissue is controlled by an estrogen response element (ERE) located 1.5 kb upstream from the transcriptional start site. To assess the ability of constitutive VEGF to block tumor regression in an inducible RAS melanoma model, mice were implanted with VEGF-expressing tumors and sustained high mortality and morbidity that were out of proportion to the tumor burden were found. Documented elevated serum levels of VEGF were associated with a lethal hepatic syndrome characterized by massive sinusoidal dilation and endothelial cell proliferation and apoptosis. Systemic levels of VEGF correlated with the severity of liver pathology and overall clinical compromise. A striking reversal of VEGF-induced liver pathology and prolonged survival were achieved by surgical excision of VEGF-seoreting tumor or by systemic administration of a potent VEGF antagonist, thus defining a paraneoplastic syndrome caused by excessive VEGF activity. Moreover, this VEGF-induced syndrome resembles peliosis hepatis, a rare human condition that is encountered in the setting of advanced malignancies, high-dose androgen therapy, and Bartonella henselae infection. Anti-VEGF therapy may be useful in the treatment of peliosis hepatis associated with excessive tumor burden or the underlying malignancy.

VEGF is a potent stimulator of endothelial cell proliferation that has been implicated in tumor growth of thyroid carcinomas. VEGF immunostaining score is a helpful marker for metastasis spread in differentiated thyroid cancers. Levels above a certain threshold value are considered as high risk for metastasis threat, prompting the physician to institute a tight follow-up of the patient. Moreover, in a thyroid carcinoma cell lines IGF1 upregulates VEGF mRNA expression and protein secretion. Transfection with vector expressing a constitutively active form of AKT, a major mediator of IGF1 signaling, also stimulates VEGF expression. The IGF1-induced upregulation of VEGF production is associated with activation of API and HIF1-alpha and was abrogated by phosphatidylinositol 3-kinase inhibitors, a JUN kinase inhibitor, HIF1-alpha antisense oligonucleotide, or geldanamycin, an inhibitor of the heat shock protein-90 molecular chaperon, which regulates the 3-dimensional conformation and function of IGF1 receptor and AKT.

Inactivation of the tumor suppressor gene PTEN and overexpression of VEGF are 2 of the most common events observed in high-grade malignant gliomas. Transfer of PTEN to glioma cells under normoxic conditions decreased the level of secreted VEGF protein by 42 to 70% at the transcriptional level. Assays suggested that PTEN acts on VEGF most likely via downregulation of the transcription factor HIF1-alpha and by inhibition of PI3K. Increased PTEN expression also inhibited the growth and migration of glioma-activated endothelial cells in culture.

Placental growth factor (PGF) regulates inter- and intramolecular cross-talk between the VEGF receptor tyrosine kinases FLT1 and FLK1. Activation of FLT1 by PGF resulted in intermolecular transphosphorylation of FLK1, thereby amplifying VEGF-driven angiogenesis through FLK1. Even though VEGF and PGF both bind FLT1, PGF uniquely stimulates the phosphorylation of specific FLT1 tyrosine residues and the expression of distinct downstream target genes. Furthermore, the VEGF/PGF heterodimer activated intramolecular VEGF receptor cross-talk through formation of FLK1/FLT1 heterodimers. The inter- and intramolecular VEGF receptor cross-talk is likely to have therapeutic implications, as treatment with VEGF/PGF heterodimer or a combination of VEGF plus PGF increased ischemic myocardial angiogenesis in a mouse model that was refractory to VEGF alone.

FGFB and VEGF differentially activate Raf1, resulting in protection from distinct pathways of apoptosis in human endothelial cells. FGFB activates Raf1 via p21-activated protein kinase-1 (PAK1) phosphorylation of serines 338 and 339, resulting in Raf1 mitochondrial translocation and endothelial cell protection from the intrinsic pathway of apoptosis, independent of the mitogen-activated protein kinase kinase-1 (MEK1). In contrast, VEGF activates Raf1 via Src kinase (CSK), leading to phosphorylation of tyrosines 340 and 341 and MEK1-dependent protection from extrinsic-mediated apoptosis. Therefore RAF1 may be a pivotal regulator of endothelial cell survival during angiogenesis.

### EGFR Family

The activity of epidermal growth factor (EGF) and its receptor the EGFR7 has been identified as key drivers in the process of cell growth and replication. Heightened activity at the EGF receptor, whether caused by an increase in the concentration of ligand around the cell, an increase in receptor numbers, a decrease in receptor turnover or receptor mutation can lead to an increase in the drive for the cell to replicate. EGFR-mediated drive is increased in a wide variety of solid tumors including non-small cell lung cancer, prostate cancer, breast cancer, gastric cancer, colorectal cancer and tumors of the head and neck. Furthermore, excessive activation of EGFR on the cancer cell surface is discussed to be associated with advanced disease, the development of a metastatic phenotype and a poor prognosis in cancer patients. Understanding how increased EGFR-mediated signalling can lead to the rapid and uncontrolled cell division characteristic of cancer is an important focus of current research—raising the possibility of new therapeutic options for the control of cancer within our reach.

The EGFR is a transmembrane receptor with an extracellular ligand-binding domain, a helical transmembrane domain, and an intracellular tyrosine kinase domain. Activation of EGFR by epidermal growth factor (EGF) and other ligands (e.g. amphiregulin, TGF-a) which bind to its extracellular domain is the first step in a series of complex signalling pathways which take the message to proliferate from the cell membrane to the genetic material deep within the cell nucleus. The EGFR is part of a subfamily of four closely related receptors EGFR (or ERBB1), Her-2/neu (ERBB2), Her-3 (ERBB3) and Her-4 (ErbB-4). Receptors exist as inactive single units or monomers that, on activation by ligand binding, pair to form an active dimer. The two receptors that form a pair are not necessarily identical, for example an EGF-1 receptor (EGFR) may pair with another EGF-1 receptor, giving a so-called homodimer, or an EGFR may pair with another member of the receptor family, such as Her 2/neu, to give an asymmetrical heterodimer. Once pairing takes place, the tyrosine kinase enzyme in the intracellular domain of the receptor becomes activated, transphosphorylating both intracellular domains, and initiating the cascade of intracellular events which results in the signal reaching the nucleus. Once activated the EGF receptor recruits a variety of proteins from the cell cytoplasm to form a linked complex. The interactions between the proteins in this activated receptor complex trigger the next step in the signalling pathway—the activation of a protein called ras which, in turn, initiates a cascade of phosphorylations which activate mitogen activated protein kinase (MAPK). MAP kinase takes the signal through the cytoplasm to the nucleus where it triggers events, which drive resting cells into cell division.

Proliferation: In the cell nucleus, two sets of molecules are crucial to orchestrating the advance of the cell through the phases of cell division: the cyclins and the cyclin-dependent kinases or cdks. Without their cyclin partners, the cdks are inactive. Once physically associated with the cdks, however, the cyclins move the cell out of its resting phase and activate the cell division process. One of these crucial cyclins, cyclin D, plays a particularly important role in this process. It is the accumulation of cyclin D that forms the last step in the pathway linking EGF receptor activation and cell division. When MAPK enters the nucleus of the cell, it triggers accumulation of cyclin D which, in association with the cdks, overcomes the “biological brake” holding the cell in its resting phase. Once the ‘biological brake’ is inactivated, the cell moves irrevocably into the active phases of division. Increased EGFR-mediated signalling can ultimately contribute to a cell moving into a state of continuous, uncontrolled cell division; the population of malignant cells expands and tumor mass increases. Traditional models of ligand-receptor interactions envisaged a linear pathway of intracellular signals linking receptor activation to a single discrete cell response. The cellular events which flow from activated membrane receptors are highly complex with several different pathways being regulated simultaneously. Increased EGFR-mediated signalling, however, is important to a number of other processes that are crucial for a number of other biological features of tumour progression such as apoptosis, angiogenesis and metastatic spread.

Apoptosis: Apoptosis is a homeostatic process which ensures that abnormal cells (old, mutated or damaged) die or are killed. In cancer cells this mechanism appears frequently to be disrupted, malignant cells do not die but, instead, continue to proliferate. Research now suggests that heightened EGFR-mediated signalling in cancer cells may play a role in blocking the normal process of apoptosis allowing abnormal cells live on to replicate and spread. The molecular processes involved in this event are not yet well understood, but it has been shown that treating cancer cells with EGFR antibodies or EGFR tyrosine kinase inhibitors promotes apoptosis and so shrinks the tumor.

Angiogenesis: Cancer cells, like all cells, need oxygen and nutrition from the blood and a rapidly increasing mass of tissue can have problems with its blood supply. Without an adequate supply of blood, a proliferating cell mass can increase to only a few hundreds of cells in size. A key strategy evolved by cancer cells to overcome this hurdle is to induce angiogenesis (the development of new vasculature from adjacent host vessels) by secreting angiogenic factors. Heightened EGFR-mediated signalling in cancer cells is linked to the increased production of some of these factors, including vascular endothelial growth factor (VEGF), a powerful stimulant of angiogenesis.

Metastasis: Increased EGFR-mediated signalling is associated with poor prognostic indicators, including a higher incidence of metastatic disease. EGFR activation promotes the ability of tumour cells to invade neighbouring tissues, especially the vascular endothelium, thus giving access to the circulation. Trapped in capillaries at a distant site tumour cells can pass out of the vessel and can establish metastases. However, the role of EGFR and its interaction with other pathways involved in these processes is not yet so well established. In summary, heightened EGFR-mediated signalling within a cancer cell may be an important factor in promoting tumour cell growth, blocking apoptosis and facilitating the processes of metastasis in several different ways. If these processes can be modified or curtailed there could be profound implications for the treatment of cancer.

### EGFR

EGFR is located on a 110-kb locus encoding the human EGF receptor and the regulation of EGF receptor expression encoded therein on human chromosome 7p13-q22 region, EGF enhances phosphorylation of several endogenous membrane proteins, including EGF receptor. The EGF receptor is a tyrosine protein kinase. It has 2 components of different molecular weight; both contain phosphotyrosine and phosphothreonine but only the higher molecular weight form contains phosphoserine. The EGFR molecule has 3 regions: one projects outside the cell and contains the site for binding EGF; the second is embedded in the membrane; the third projects into the cytoplasm of the cell's interior. EGFR is a kinase that attaches phosphate groups to tyrosine residues in proteins. EGFR signaling involves small GTPases of the Rho family, and EGFR trafficking involves small GTPases of the Rab family. EPS8 protein connects these signaling pathways, EPS8 is a substrate of EGFR that is held in a complex with SOS1 by the adaptor protein E3B1, thereby mediating activation of RAC. Through its SH3 domain, EPS8 interacts with RNTRE, which in turn is a RAB5 GTPase-activating protein whose activity is regulated by EGFR. By entering in a complex with EPS8, RNTRE acts on RAB5 and inhibits internalization of the EGFR. Furthermore, RNTRE diverts EPS8 from its RAC-activating function, resulting in the attenuation of RAC signaling. Thus, depending on its state of association with E3B1 or RNTRE, EPS8 participates in both EGFR signaling through RAC and EGFR trafficking through RAB5. There is evidence for a novel signaling mechanism consisting of ligand-independent lateral propagation of receptor activation in the plasma membrane. Phosphorylation of green fluorescent protein-tagged ERBB1 receptors in cells focally stimulated with EGF covalently attached to beads. The rapid and extensive propagation of receptor phosphorylation over the entire cell after focal stimulation demonstrated a signaling wave at the plasma membrane resulting in full activation of all receptors.

Treatment with genistein, an inhibitor of tyrosine kinase activity, inhibits EGF-induced tyrosine phosphorylation and degradation of EGFR in cancer cell lines, suggesting that tyrosine kinase activity is required for either the internalization or the degradation of EGF-EGFR receptor complexes. It has been found that the oncogene ERBB is derived from the gene coding for EGFR. Strikingly, the most consistent chromosomal finding in a series of human glioblastoma cell lines was an increase in copy number of chromosome 7. Accordingly, ERBB-specific mRNAs are increased to levels even higher than expected from the number of chromosomes 7 present. These changes were not found in benign astrocytomas.

In a multiinstitutional phase II trial, a higher rate of response to the tyrosine kinase inhibitor gefitinib (Iressa) in Japanese patients with nonsmall cell lung cancer than in a predominantly European-derived population (27.5% vs 10.4%). Most patients with NSCLC have no response to gefitinib, which targets the epidermal growth factor receptor. However, approximately 10% of patients have a rapid and often dramatic clinical response. Somatic mutations in the tyrosine kinase domain of the EGFR gene were found in 8 of 9 patients with gefitinib-responsive lung cancer as compared with none of the 7 patients with no response (P less than 0.001). Mutations were either small in-frame deletions or amino acid substitutions that were clustered around the ATP-binding pocket of the tyrosine kinase domain. Similar mutations were detected in tumors from 2 of 25 patients (8%) with primary NSCLC who had not been exposed to gefitinib. All mutations were heterozygous, and identical mutations were observed in multiple patients, suggesting an additive specific gain of function. In vitro, EGFR mutations demonstrated enhanced tyrosine kinase activity in response to epidermal growth factor and increased sensitivity to inhibition by gefitinib. Somatic mutations in EGFR were found in 15 of 58 unselected NSCLC tumors from Japan and 1 of 61 from the United States. EGFR mutations showed a striking correlation with patient characteristics. Mutations were more frequent in adenocarcinomas than in other NSCLCs, being present in 15 of 70 (21%) and 1 of 49 (2%), respectively; more frequent in women than in men, being present in 9 of 45 (20%) and 7 of 74 (9%), respectively; and more frequent in patients from Japan than in those from the United States, being present in 15 of 58 (26%) and 14 of 41 adenocarcinomas (32%) versus 1 of 61 (2%) and 1 of 29 adenocarcinomas (3%), respectively. The patient characteristics that correlated with the presence of EGFR mutations were those that correlated with clinical response to gefitinib treatment. It has been suggested that identification of EGFR mutations in other malignancies, perhaps including glioblastomas in which EGFR alterations had previously been identified (Yamazaki et al. 1988), may identify other patients who would similarly benefit from treatment with EGFR inhibitors. The striking difference in the frequency of EGFR mutation and response to gefitinib between Japanese and U.S. patients raised general questions regarding variation in the molecular pathogenesis of cancer in different ethnic, cultural, and geographic groups and argued for the benefit of population diversity in cancer clinical trials.

EGFR is required for skin development and is implicated in epithelial tumor formation. Transgenic mice expressing SOS-F (a dominant form of ‘son of sevenless’ (SOS1) lacking the C-terminal region containing the GRB2-binding site and instead carrying the c-Ha-ras farnesylation site, which provides constitutive activity) driven by the keratin-5 (K5, or KRT5) promoter in basal keratinocytes developed skin papillomas with 100% penetrance. Tumor formation was inhibited, however, in mice with a hypomorphic and null Egfr background. Similarly. Egfr-deficient fibroblasts were resistant to transformation by SOS-F and rasV12, although tumorigenicity could be restored by expression of the antiapoptotic Bcl2 gene. The K5-SOS-F papillomas and primary keratinocytes displayed increased apoptosis and reduced Akt phosphorylation, and grafting experiments implied a cell-autonomous requirement for Egfr in keratinocytes. Therefore, the authors concluded that EGFR functions as a survival factor in oncogenic transformation and provides a valuable target for therapeutic intervention.

Activation of epidermal growth factor receptor triggers mitogenic signaling in gastrointestinal mucosa, and its expression is also upregulated in colon cancers and most neoplasms. It has been investigated whether prostaglandins transactivate EGFR. Prostaglandin E2 (PGE2) rapidly phosphorylates EGFR and triggers the extracellular signal-regulated kinase 2 (ERK2)-mitogenic signaling pathway in normal gastric epithelial and colon cancer cell lines. Inactivation of EGFR kinase with selective inhibitors significantly reduced PGE2-induced ERK2 activation, c-fos mRNA expression, and cell proliferation. Inhibition of matrix metalloproteinases, TGFA, or c-Src blocked PGE2-mediated EGFR transactivation and downstream signaling, indicating that PGE2-induced EGFR transactivation involves signaling transduced via TGF-alpha, an EGFR ligand, likely released by c-Src-activated MMPs.

### Her-2/neu

The oncogene originally called NEU was derived from rat neuro/glioblastoma cell lines. It encodes a tumor antigen, p185, which is serologically related to EGFR, the epidermal growth factor receptor, EGFR maps to chromosome 7. In 1985 it was found, that the human homologue, which they designated NGL (to avoid confusion with neuraminidase, which is also symbolized NEU), maps to 17q12-q22 by in situ hybridization and to 17q21-qter in somatic cell hybrids. Thus, the SRO is 17q21-q22. Moreover, in 1985 a potential cell surface receptor of the tyrosine kinase gene family was identified and characterized by cloning the gene. Its primary sequence is very similar to that of the human epidermal growth factor receptor. Because of the seemingly close relationship to the human EGF receptor, the authors called the gene HER2. By Southern blot analysis of somatic cell hybrid DNA and by in situ hybridization, the gene was assigned to 17q21-q22. This chromosomal location of the gene is coincident with the NEU oncogene, which suggests that the 2 genes may in fact be the same; indeed, sequencing indicates that they are identical. In 1988 a correlation between overexpression of NEU protein and the large-cell, comedo growth type of ductal carcinoma was found. The authors found no correlation, however, with lymph-node status or tumor recurrence. The role of HER2/NEU in breast and ovarian cancer was described in 1989, which together account for one-third of all cancers in women and approximately one-quarter of cancer-related deaths in females.

An ERBB-related gene that is distinct from the ERBB gene, called ERBB1 was found in 1985. ERBB2 was not amplified in vulva carcinoma cells with EGFR amplification and did not react with EGF receptor mRNA. About 30-fold amplification of ERBB2 was observed in a human adenocarcinoma of the salivary gland. By chromosome sorting combined with velocity sedimentation and Southern hybridization, the ERBB2 gene was assigned to chromosome 17. By hybridization to sorted chromosomes and to metaphase spreads with a genomic probe, they mapped the ERBB2 locus to 17q21. This is the chromosome 17 breakpoint in acute promyelocyte leukemia (APL). Furthermore, they observed amplification and elevated expression of the ERBB2 gene in a gastric cancer cell line. Antibodies against a synthetic peptide corresponding to 14 amino acid residues at the COOH-terminus of a protein deduced from the ERBB2 nucleotide sequence were raised in 1986. With these antibodies, the ERBB2 gene product from adenocarcinoma cells was precipitated and demonstrated to be a 185-kD glycoprotein with tyrosine kinase activity. A cDNA probe for ERBB2 and by in situ hybridization to APL cells with a 15;17 chromosome translocation located the gene to the proximal side of the breakpoint. The authors suggested that both the gene and the breakpoint are located in band 17q21.1 and, further, that the ERBB2 gene is involved in the development of leukemia. In 1987 experiments indicated that NEU and HER2 are both the same as ERBB2. The authors demonstrated that overexpression alone can convert the gene for a normal growth factor receptor, namely, ERBB2, into an oncogene. The ERBB2 to 17q11-q21 by in situ hybridization. By in situ hybridization to chromosomes derived from fibroblasts carrying a constitutional translocation between 15 and 17, they showed that the ERBB2 gene was relocated to the derivative chromosome 15; the gene can thus be localized to 17q12-q21.32. By family linkage studies using multiple DNA markers in the 17q12-q21 region the ERBB2 gene was placed on the genetic map of the region.

Interleukin-6 is a cytokine that was initially recognized as a regulator of immune and inflammatory responses, but also regulates the growth of many tumor cells, including prostate cancer. Overexpression of ERBB2 and ERBB3 has been implicated in the neoplastic transformation of prostate cancer. Treatment of a prostate cancer cell line with IL6 induced tyrosine phosphorylation of ERBB2 and ERBB3, but not ERBB1/EGFR. The ERBB2 forms a complex with the gp130 subunit of the IL6 receptor in an IL6-dependent manner. This association was important because the inhibition of ERBB2 activity resulted in abrogation of IL6-induced MAPK activation. Thus, ERBB2 is a critical component of IL6 signaling through the MAP kinase pathway. These findings showed how a cytokine receptor can diversify its signaling pathways by engaging with a growth factor receptor kinase.

Overexpression of ERBB2 confers Taxol resistance in breast cancers. Overexpression of ERBB2 inhibits Taxol-induced apoptosis. Taxol activates CDC2 kinase in MDA-MB-435 breast cancer cells, leading to cell cycle arrest at the G2/M phase and, subsequently, apoptosis. A chemical inhibitor of CDC2 and a dominant-negative mutant of CDC2 blocked Taxol-induced apoptosis in these cells. Overexpression of ERBB2 in MDA-MB-435 cells by transfection transcriptionally upregulates CDKN1A which associates with CDC2, inhibits Taxol-mediated CDC2 activation, delays cell entrance to G2/M phase, and thereby inhibits Taxol-induced apoptosis. In CDKN1A antisense-transfected MDA-MB-435 cells or in p21 −/− MEF cells, ERBB2 was unable to inhibit Taxol-induced apoptosis. Therefore, CDKN1A participates in the regulation of a G2/M checkpoint that contributes to resistance to Taxol-induced apoptosis in ERBB2-overexpressing breast cancer cells.

A secreted protein of approximately 68 kD was described, designated herstatin, as the product of an alternative ERBB2 transcript that retains intron 8. This alternative transcript specifies 340 residues identical to subdomains I and II from the extracellular domain of p185ERBB2, followed by a unique C-terminal sequence of 79 amino acids encoded by intron 8. The recombinant product of the alternative transcript specifically bound to ERBB2-transfected cells and was chemically crosslinked to p185ERBB2, whereas the intron-encoded sequence alone also bound with high affinity to transfected cells and associated with p185 solubilized from cell extracts. The herstatin mRNA was expressed in normal human fetal kidney and liver, but was at reduced levels relative to p185ERBB2 mRNA in carcinoma cells that contained an amplified ERBB2 gene. Herstatin appears to be an inhibitor of p185ERBB2, because it disrupts dimers, reduces tyrosine phosphorylation of p185, and inhibits the anchorage-independent growth of transformed cells that overexpress ERBB2. The HER2 gene is amplified and HER2 is overexpressed in 25 to 30% of breast cancers, increasing the aggressiveness of the tumor. Finally, it was found that a recombinant monoclonal antibody against HER2 increased the clinical benefit of first-line chemotherapy in metastatic breast cancer that overexpresses HER2.

### ERBB3

In 1989 a DNA fragment related to but distinct from epidermal growth factor receptor EGFR and ERBB2 was detected. cDNA cloning showed a predicted 148-kD transmembrane polypeptide with structural features identifying it as a member of the ERBB gene family, prompting the designation ERBB3. Markedly elevated ERBB3 mRNA levels were demonstrated in certain human mammary tumor cell lines, suggesting that it may play a role in some human malignancies just as does EGFR (also called ERBB1). Epidermal growth factor, transforming growth factor alpha and amphiregulin are structurally and functionally related growth regulatory proteins. They all are secreted polypeptides that bind to the 170-kD cell-surface EGF receptor, activating its intrinsic kinase activity. These 3 proteins differentially interact with a homolog of EGFR. They failed to show any interaction between these 3 secreted growth factors and ERBB2, a known EGFR-related protein. Searching for other members of this family of receptor tyrosine kinases, however, they cloned and studied the expression of ERBB3, which they referred to as HER3. The cDNA was isolated from a human carcinoma cell line, and its 6-kb transcript was identified in various human tissues. ERBB3 is a receptor for heregulin and is capable of mediating HGL-stimulated tyrosine phosphorylation of itself. The 2.6-angstrom crystal structure of the entire extracellular region of human HER3 has been determined. The structure consists of 4 domains with structural homology to domains found in the type I insulin-like growth factor receptor. The HERB structure revealed a contact between domains n and TV that constrains the relative orientations of ligand-binding domains and provides a structural basis for understanding both multiple-affinity forms of EGFRs and conformational changes induced in the receptor by ligand binding during signaling. By in situ hybridization ERBB3 gene has been mapped to chromosome 12q13.

### ERBB4

The HER4/ERBB4 gene is a member of the type I receptor tyrosine kinase subfamily that includes EGFR, ERBB2, and ERBB3. It encodes a receptor for NDF/heregulin (NRG1). Using in situ hybridization and immunohistochemical analysis, it was shown that Erbb4 was extensively expressed in adult and fetal mouse tissues. Expression was strong in the lining epithelia of the gastrointestinal, urinary, reproductive, and respiratory tracts, as well as in skin, skeletal muscle, circulatory, endocrine, and nervous systems. The developing brain and heart expressed high levels of Erbb4. Neuregulins and their receptors, the ERBB protein tyrosine kinases, are essential for neuronal development. ERBB4 is enriched in the postsynaptic density and associates with PSD95. Heterologous expression of PSD95 enhanced NRG activation of ERBB4 and MAP kinase. Conversely, inhibiting expression of PSD95 in neurons attenuated NRG-mediated activation of MAP kinase. PSD95 formed a ternary complex with 2 molecules of ERBB4, suggesting that PSD95 facilitates ERBB4 dimerization. Finally, NRG suppressed induction of long-term potentiation in the hippocampal CA1 region without affecting basal synaptic transmission. Thus, NRG signaling may be synaptic and regulated by PSD95. The role of NRG signaling in the adult central nervous system may be modulation of synaptic plasticity. ERBB4 and PSD95 coimmunoprecipitated from rat forebrain lysates and that the direct interaction was mediated through the C-terminal end of ERBB4. Immunofluorescent studies of cultured rat hippocampal cells showed that ERBB4 colocalized with PSD95 and NMDA receptors at interneuronal postsynaptic sites. The findings suggested that certain ERBB receptors interact with other receptors and may be important in activity-dependent synaptic plasticity. ERBB4 is a transmembrane receptor tyrosine kinase that regulates cell proliferation and differentiation. After binding its ligand, heregulin, or activation of protein kinase C by TPA, the ERBB4 ectodomain is cleaved by a metalloprotease. Subsequent cleavage by gamma-secretase that releases the ERBB4 intracellular domain from the membrane and facilitates its translocation to the nucleus. Gamma-secretase cleavage was prevented by chemical inhibitors or a dominant-negative presenilis Inhibition of gamma-secretase also prevented growth inhibition by heregulin. Gamma-secretase cleavage of ERBB4 may represent another mechanism for receptor tyrosine kinase-mediated signaling. Using human cDNA probes in fluorescence in situ hybridization the ERBB4 gene has been mapped to chromosome 2q33.3-q34. The finding established that the ERBB4 gene, like the related EGFR, ERBB2, and ERBB3 genes, is located in close proximity to homeobox and collagen gene loci. ErbB4 −/− mouse embryos develop trigeminal ganglion and geniculate/cochleovestibular ganglia that are displaced toward each other and show axonal misprojections. These morphologic changes correlate with aberrant migration of a subpopulation of hindbrain-derived cranial neural crest cells. The aberrant migration is also accompanied by an apparent downregulation of HoxB2 gene expression. Through transplantation experiments, it was determined that neural crest cells deviated from their normal pathway only when transplanted into mutant embryos, suggesting that ErbB4 signaling within the host environment provides patterning information essential for the proper migration of neural crest cells. Transgenic mice were generated that expressed a dominant-negative ErbB4 receptor specifically in nonmyelinating Schwann cells. The mutant mice developed a progressive peripheral neuropathy characterized by extensive Schwann cell proliferation and death, loss-of unmyelinated axons, and marked hot and cold pain insensitivity. At later stages, the mutant mice showed a loss of C-fiber dorsal root ganglion neurons. The findings indicated that the NRG1-ErbB4 signaling system contributes to reciprocal interactions between unmyelinated sensory axons and nonmyelinating Schwann cells that appear to be critical for Schwann cell and C-fiber sensory neuron survival. ERBB4 was expressed at high levels in neural precursor cells in the rat subventricular zone (SVZ) and rostral migratory system (RMS) that are destined to become olfactory interneurons. ERBB4 was also detected in a subset of glial cells. Mice with targeted deletion of the ErbB4 gene in the CNS showed cellular disorganization of the SVZ and RMS as well as altered distribution and differentiation of olfactory interneurons. In vivo, cells explanted from mutant mice failed to form migratory neuronal chains and showed impaired orientation compared to wildtype cells. It has been concluded that ERBB4 plays a role in RMS neuroblast tangential migration and olfactory interneuronal placement.

Mice lacking neural Erbb4 expression had reduced numbers of GABA-positive neurons in the postnatal cortex and hippocampus. Nrg1 is a neural guidance molecule for GABAergic interneurons from the medial ganglionic eminence. Thus, the loss of GABAergic neurons in Erbb4 mutant mice attributed to abnormal migration of these interneurons to the neocortex.

### Metabolism Motif

### Malic Enzymes

NADP(+)-dependent malic enzyme catalyzes the reversible oxidative decarboxylation of malate and is a link between the glycolytic pathway and the citric acid cycle. The reaction is L-malate plus NADP(+) to form pyruvate, CO(2) and NADPH. There are 2 types of NADP(+)-dependent malic enzymes, a cytosolic form (ME1) and a mitochondrial form (ME3). These enzymes are also called NADP(+)-dependent malate dehydrogenases. ME2, which is NAD(+)-dependent, is a third type of malic enzyme. The soluble malic enzyme and a mitochondrial form of malic enzyme are tetrameric. The predicted ME1 protein contains 572 amino acids and has a calculated molecular mass of 64.1 kD. The human ME1 protein is 89% identical to mouse and rat MeI, 77% identical to duck ME1, and 54% identical to human ME2. The 5-prime flanking region of the human ME1 gene harbors 2 regions that mediate positive transcriptional regulation by triiodothyronine (T3). Therefore hormones such as T3 appears to control ME1 transcription by inducing both the dissociation of thyroid hormone receptor-beta (THRB) homodimers and the functional activation of ligand-bound heterodimers. Computer analysis revealed the presence of additional putative recognition motifs for numerous transcription factors and hormone receptors, which suggests that the ME1 gene is under complex regulatory control. Nonidentity of the cancer cell malic enzyme to that from the normal human cell has been discussed.

### AKRIC1

Aldo-keto reductase family member 1 (AKR1C1) is also called dihydrodiol dehydrogenase type 1 odr aldo-ketoreductase. It belongs to the aldo-keto reductase superfamily, which also includes aldehyde reductase, aldose reductase, 3-alpha-hydroxysteroid dehydrogenase (3-alpha-HSD), and several other closely related proteins. These enzymes catalyze the conversion of aldehydes and ketones to their corresponding alcohols by utilizing NADH and/or NADPH as cofactors and exist in cellular cytoplasm as monomeric 34- to 36-kD proteins. The enzymes display overlapping but distinct substrate specificity. The importance of dihydrodiol dehydrogenase activity in the detoxification of polycystic aromatic hydrocarbons was demonstrated by its ability to reduce the mutagenic activity of benzo[a]pyrene in the Ames test. Chlordecone reductase is the enzyme involved in the detoxification of organochloride pesticides. The gene spans approximately 16 kb and consists of 9 exons. Several additional hybridizing DNA bands have been found by northern blotting techniques, suggesting the existence of multiple related genes.

### PPARG

The peroxisome proliferator-activated receptors (PPARs) are members of the nuclear hormone receptor subfamily of transcription factors. PPARs form heterodimers with retinoid X receptors (RXRs) and these heterodimers regulate transcription of various genes. There are 3 known subtypes of PPARs, PPAR-alpha, PPAR-delta and PPAR-gamma. PPAR-gamma (=PPARG) is believed to be involved in adipocyte differentiation, showed that PPAR-gamma is expressed at significant levels in human primary and metastatic breast adenocarcinomas. Ligand activation of this receptor in cultured breast cancer cells caused extensive lipid accumulation, changes in breast epithelial gene expression associated with a more differentiated, less malignant state, and a reduction in growth rate and clonogenic capacity of the cells. Inhibition of MAP kinase, a powerful negative regulator of PPAR-gamma, improves the TZD ligand sensitivity of nonresponsive cells. These data suggested that the PPAR-gamma transcriptional pathway can induce terminal differentiation of malignant breast epithelial cells. PPARG is involved in the regulation metabolic activities of diseased and non-diseased tissues. For examples it is involved in the regulation of the healthy adipose tissue but also involved in the formation of foam cells from macrophages in the aterial wall during aterosclerotic lesions. Natural and synthetic agonists of PPAR-gamma regulate adipocyte differentiation, glucose homeostasis, and inflammatory responses. PPAR-gamma is expressed in human prostate adenocarcinomas and cell lines derived from these tumors. Activation of this receptor with specific ligands exerts an inhibitory effect on the growth of prostate cancer cell lines. Prostate cancer tumors and cell lines do not have intragenic mutations in the PPARG gene, although 40% of the informative tumors have hemizygous deletions of this gene. Oral treatment advanced prostate cancer with troglitazone (Rezulin), a PPAR-gamma ligand used for the treatment of type II diabetes, was administered to 41 men with histologically confirmed prostate cancer and no symptomatic metastatic disease. An unexpectedly high incidence of prolonged stabilization of prostate-specific antigen (KLK3) was seen in patients treated with troglitazone. In addition, 1 patient had a dramatic decrease in serum prostate-specific antigen to nearly undetectable levels. The findings suggested that PPAR-gamma may serve as a biologic modifier in human prostate cancer and that its therapeutic potential should be studied further. RT-PCR and immunocytochemical analysis demonstrated that the malignant T cell lines, but not normal resting T cells, expressed PPARG mRNA as well as cytoplasmic and nuclear PPARG protein. In addition, PPARG agonists, but not PPARA agonists, mimicked the action of PGD2 and its metabolite, 15-d-PGJ2, in inhibiting the proliferation and viability of the T-cell tumor lines and in inducing apoptosis in these cells. Therefore PPARG ligands, which may include PGD2, provide strong apoptotic signals to transformed but not normal T lymphocytes. Adrenocorticotrophic hormone (ACTH)-secreting pituitary tumors are associated with high morbidity due to excess glucocorticoid production. PPAR-gamma protein is expressed exclusively in normal ACTH-secreting human anterior pituitary cells. PPAR-gamma activators induced G0/G1 cell cycle arrest and apoptosis and suppressed ACTH secretion in human and murine corticotroph tumor cells. Development of murine corticotroph tumors, generated by subcutaneous injection of ACTH-secreting AtT20 cells, was prevented in 4 of 5 mice treated with the TZD compound rosiglitazone, and ACTH and corticosterone secretion was suppressed in all treated mice. Based on these findings TZDs may be an effective therapy for Cushing disease.

### PLCB4

In the phosphoinositide (PI) cycle, phospholipase C (PLC) catalyzes hydrolysis of a plasma membrane phospholipid, phosphatidylinositol 4,5-biphosphate, generating 2 second messengers, the water soluble 1,4,5-inositol trisphosphate and the membrane-associated 1,2-diacylglycerol. In mammalian tissues, 3 groups of PLCs have been characterized, termed beta (e.g., PLCB3), gamma (e.g., PLCG1), and delta, (e.g., PLCD1) and each group consists of at least 3 isoforms. These proteins are single polypeptides, ranging in molecular mass from 65 to 154 kD. Several lines of evidence suggested signal transduction via the PI cycle plays a role in the light response in vertebrate and invertebrate retinas. Defects in the Drosophila norpA (‘no receptor potential A’) gene encoding a phosphoinositide-specific PLC block invertebrate phototransduction and lead to retinal degeneration. Phospholipase C beta-4 is expressed in the suprachiasmatic nucleus (SCN) in the mouse. PLCB4 −/− mice had a pronounced loss of persistent circadian rhythm under constant darkness and a significantly decreased spontaneous firing rate of suprachiasmatic neurons during the subjective day. Antagonist studies showed that PLCB4 is coupled to metabotropic glutamate receptors in the SCN, and that this signaling pathway is involved in translating circadian oscillations of the molecular clock into rhythmic outputs of SCN neurons.

### Apoptosis/Signaling Motif

### MAP3K5

Mitogen-activated protein kinase (MAPK) signaling cascades include MAPK or extracellular signal-regulated kinase (ERK), MAPK kinase (MAP2K, also called MKK or MEK), and MAPK kinase kinase (MAP3K, also called MAPKKK or MEKK). MAPKK kinase/MEKK phosphorylates and activates its downstream protein kinase, MAPK kinase/MEK, which in turn activates MAPK. The kinases of these signaling cascades are highly conserved, and homologs exist in yeast, Drosophila, and mammalian cells.

The MAP3K5 protein contains 1,374 amino acids with all 11 kinase subdomains. MAP3K5 transcript is abundantly expressed in human heart and pancreas. The MAP3K5 protein phosphorylates and activates MKK4 in vitro, and activates c-jun N-terminal kinase (JNK)/stress-activated protein kinase. MAP3K5 does not activate MAPK/ERK. A nearly identical MAP3K5 cDNA, termed ASK1 for apoptosis signal-regulating kinase has been identified. The deduced protein contains 1,375 amino acids, and is most closely related to yeast SSK2 and SSK22, which are upstream regulators of yeast HOG 2 MAPK. ASK1 expression complements a yeast mutant lacking functional SSK2 and SSK22. ASK1 also activates MKK3, MKK4 (SEK1), and MKK6. Overexpression of ASK1 induces apoptotic cell death, and ASK1 is activated in cells treated with tumor necrosis factor-alpha (TNFA). ASK1 interacts with members of the TRAP family and is activated by TRAF2 in the TNF-signaling pathway. After activation by TRAF2, ASK1 activates MKK4, which in turn activates INK. Thus, ASK1 is a mediator of TRAF2-induced JNK activation. A virulence factor from Yersinia pseudotuberculosis, YopJ, is a 33-kD protein that perturbs a multiplicity of signaling pathways. These include inhibition of the extracellular signal-regulated kinase ERK, c-jun NH2-terminal kinase (JNK) and p38 mitogen-activated protein kinase pathways and inhibition of the nuclear factor kappa B pathway. The expression of YopJ has been correlated with the induction of apoptosis by Yersinia. Mammalian binding partners of YopJ include MAPK kinases MKK1, MKK2, and MKK4/SEK1. YopJ was found to bind directly to MKKs in vitro, including MKK1, MKK3, MKK4, and MKK5. Binding of YopJ to the MKK blocked both phosphorylation and subsequent activation of the MKKs. These results explain the diverse activities of YopJ in inhibiting the ERIC, JNK, p38, and NF-kappa-B signaling pathways, preventing cytokine synthesis and promoting apoptosis.

JNK3 is a binding partner of beta-arrestin-2 (ARBB2). The upstream JNK activators ASK1 and MKK4 are found in complex with ARBB2. Cellular transfection of ARBB2 caused cytosolic retention of JNK3 and enhanced JNK3 phosphorylation stimulated by ASK1. Moreover, stimulation of the angiotensin II type 1A receptor (AGTR1) activated JNK3 and triggered the colocalization of ARBB2 and active JNK3 to intracellular vesicles. ARBB2 acts as a scaffold protein, which brings the spatial distribution and activity of this MAPK module under the control of a G protein-coupled receptor.

Activity of ASK1, but not of TAK1 (MAP3K7) or an ASK1 lys709-to-arg mutant, is potentiated by coexpression with DAXX or the JNK activation domain (amino acids 501 to 625) of DAXX. FAS activation was found to enhance endogenous ASK1 activity. ASK1 directly interacts with DAXX but not FAS, indicating that DAXX acts as a bridge between FAS and ASK1. The DAXX-ASK1 connection provides a mechanism for caspase-independent activation of JNK by FAS and perhaps other stimuli. Fas triggers cell death specifically in motor neurons by transcriptional upregulation of neuronal nitric oxide synthase (nNOS) mediated by p38 kinase. ASK1 and Daxx act upstream of p38 in the Fas signaling pathway. The authors also showed that synergistic activation of the NO pathway and the classic FADD/caspase-8 pathway were needed for motor neuron cell death, No evidence for involvement of the Fas/NO pathway was found in other cell types. Motor neurons from transgenic mice expressing amyotrophic lateral sclerosis (ALS)-linked SOD1 mutations displayed increased susceptibility to activation of the Fas/NO pathway. This signaling pathway was unique to motor neurons and suggested that these cell death pathways may contribute to motor neuron loss in ALS.

### SPON 1

The deduced 624-amino acid partial SPON1 protein shares 96.8% amino acid sequence identity with the rat F-spondin precursor across 624 residues. Analysts of SPON1 expression in 10 human tissues by RT-PCR followed by ELISA detected highest SPON1 expression in lung, lower expression in brain, heart, kidney, liver, and testis, and lowest expression in pancreas, skeletal muscle, and ovary; no expression was found in spleen. Rat brain Spon1 immunoprecipitated with a critical central sequence of APP. In vitro binding assays using mutated human proteins confirmed that SPON1 specifically bound to the central APP domain (CAPPD). SPON1 inhibited APP cleavage by BACEI, the primary beta-secretase involved in APP processing. Binding also impaired APP- and FE65-dependent transactivation of the chromosome remodeling factor TIP60. By binding to the extracellular CAPPD of APP, SPON1 inhibits APP processing and thereby impairs APP-dependent transcriptional transactivation.

### PLCB4

In the phosphoinositide (PI) cycle, phospholipase C (PLC) catalyzes hydrolysis of a plasma membrane phospholipid, phosphatidylinositol 4,5-biphosphate, generating 2 second messengers, the water soluble 1,4,5-inositol trisphosphate and the membrane-associated 1,2-diacyl glycerol. In mammalian tissues, 3 groups of PLCs have been characterized, termed beta (e.g., PLCB3), gamma (e.g., PLCG1), and delta, (e.g., PLCD1) and each group consists of at least 3 isoforms. These proteins are single polypeptides, ranging in molecular mass from 65 to 154 kD. Several lines of evidence suggested signal transduction via the PI cycle plays a role in the light response in vertebrate and invertebrate retinas. Defects in the Drosophila norpA (‘no receptor potential A’) gene encoding a phosphoinositide-specific PLC block invertebrate phototransduction and lead to retinal degeneration. Phospholipase C beta-4 is expressed in the suprachiasmatic nucleus (SCN) in the mouse. PLCB4 −/− mice had a pronounced loss of persistent circadian rhythm under constant darkness and a significantly decreased spontaneous firing rate of suprachiasmatic neurons during the subjective day. Antagonist studies showed that PLCB4 is coupled to metabotropic glutamate receptors in the SCN, and that this signaling pathway is involved in translating circadian oscillations of the molecular clock into rhythmic outputs of SCN neurons.

### Aute Phase Motif

### ORM1

This serum protein, also called orosomucoid, is a monomer about 210 amino acid residues long; the amino acid sequence has been determined through 192 amino acids. The genomic DNA segment encoding orosomucoid contains 3 adjacent coding regions termed AGP-A, B, and B-prime (AGP=acid-glycoprotein). The regions were identical in exon-intron organization but had slightly different coding potentials. These results accounted for the heterogeneity observed by protein sequencing. Southern blot analysis indicated that the cloned cluster contains all the orosomucoid coding sequences present in the human genome. Most of the alpha-AGP mRNA in human liver is transcribed from AGP-A, whose promoter and cap site have been determined, while the level of AGP-B and B-prime mRNA in human liver is very low. The regulation of AGP-A was investigated by transfecting cell lines and preparing transgenic mice with constructs including the entire AGP gene. The AGP constructs were expressed with comparable efficiency in hepatoma and HeLa cells; however, these same constructs were expressed in transgenic mice in a tissue-specific manner. The mRNA was found solely in the liver. These authors found that a 6,6-kb segment consisting of the entire coding region plus 1.2 kbs of 5-prime-flanking and 2 kbs of 3-prime-flanking DNA contained sufficient information for tissue-specific, regulated expression of the gene

Variants have been demonstrated in the blood of normal Caucasians and Japanese. Data on gene frequencies of allelic variants reported a total of 57 different alleles at the ORM1 and ORM2 loci. Twenty-seven were assigned to the ORM1 locus and 30 to the ORM2 locus. In plasma, ORM proteins are presented as a mixture of ORM1 and ORM2 proteins in a molar ratio of 3:1, respectively. Classic genetic polymorphism occurs in the more abundant ORM1, which is controlled by the ORM1 locus. ORM1*F, the ‘fast’ allele, is divided into 2 subtype alleles, ORM1*F1 and ORM1*F2. ORM1*F1 and ORM1*S are observed worldwide and ORM1*F2 is also common in European populations. The ORM2 locus is monomorphic in most populations. About 30 rare variant alleles had been distinguished electrophoretically at each of the loci. The tandemly arranged genes at the ORM1 and ORM2 loci (also designated AGP1 and AGP2, respectively) span about 11.5 kb. Each gene consists of 6 exons and 5 introns and encodes a 183-amino acid polypeptide.

### APCS

In 1985 the cDNA for the P component of human serum amyloid and determined the complete sequence of the precursor has been isolated. The APCS gene is probably closely situated to that for C-reactive protein (CRP) with which it shows homology. A genetic marker for susceptibility to amyloidosis in juvenile arthritis: an 8,8-kb RFLP band determined by a polymorphic DNA site 5-prime to the APCS gene. Homozygosity for the alternative 5.6-kb band was found in none of 28 amyloid patients. Among 19 juvenile arthritic patients without amyloidosis, the distribution of the polymorphism was the same as that in the normal group. It might be significant that this region includes CRP, APCS, and histone genes, all of which have products that interact with DNA. Induction of reactive amyloidosis was retarded in mice lacking APCS, demonstrating the participation of APCS in pathogenesis of amyloidosis in vivo and confirming that inhibition of APCS binding to amyloid fibrils is an attractive therapeutic target. A drug that is a competitive inhibitor of APCS binding to amyloid fibrils has been developed. This palindromic compound also crosslinks and dimerizes APCS molecules, leading to their very rapid clearance by the liver and thus producing a marked depletion of circulating human APCS. This mechanism of drug action potently removes APCS from human amyloid deposits in tissues and may provide a new therapeutic approach to both systemic amyloidosis and diseases associated with local amyloid, including Alzheimer disease and type 2 diabetes. As for SPON 1 there could be a link to APP and Alzheimer disease.

### Polynucleotides

A “CANCER GENE” polynucleotide can be single- or double-stranded and comprises a coding sequence or the complement of a coding sequence for a “CANCER GENE” polypeptide. Degenerate nucleotide sequences encoding human “CANCER GENE” polypeptides, as well as homologous nucleotide sequences which are at least about 50, 55, 60, 65, 70, preferably about 75, 90, 96, or 98% identical to the nucleotide sequences of Table 1 also are “CANCER GENE” polynucleotides.

### Identification of Differential Expression

Transcripts within the collected RNA samples which represent RNA produced by differentially expressed genes may be identified by utilizing a variety of methods which are ell known to those of skill in the art. For example, differential screening [Tedder, T. F. et al., 1988], subtractive hybridization [Hedrick, S. M. et ah, 1984] and, preferably, differential display (Liang, P., and Pardee, A. B., 1993, U.S. Pat. No. 5,262,311, which is incorporated herein by reference in its entirety), may be utilized to identify polynucleotide sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type derived from a control subject, while the second cDNA probe may correspond to a total cell cDNA probe of the same cell type derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue, the hybridization of the mRNA or single-stranded cDNA reverse-transcribed from the Isolated mRNA, and the removal of all hybridized, and therefore double-stranded, sequences. The remaining non-hybridized, single-stranded cDNA, potentially represent clones derived from genes that are differentially expressed in the two mRNA sources. Such single-stranded cDNA is then used as the starting material for the construction of a library comprising clones derived from differentially expressed genes.

The differential display technique describes a procedure, utilizing the well known polymerase chain reaction (PCR; the experimental embodiment set forth in Mullis, K, B., 1987, U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotide described below. Next, this technique uses pairs of PCR primers, as described below, which allow for the amplification of clones representing a random subset of the RNA transcripts present within any given cell.

Utilizing different pairs of primers allows each of the mRNA transcripts present in a cell to be amplified. Among such amplified transcripts may be identified those which have been produced from differentially expressed genes.

The reverse oligonucleotide primer of the primer pairs may contain an oligo dT stretch of nucleotides, preferably eleven nucleotides long, at its 5′ end, which hybridizes to the poly(A) tail of mRNA or to the complement of a cDNA reverse transcribed from an mRNA poly(A) tail. Second, in order to increase the specificity of the reverse primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The forward primer may contain a nucleotide sequence expected, statistically, to have the ability to hybridize to cDNA sequences derived from the tissues of interest. The nucleotide sequence may be an arbitrary one, and the length of the forward oligonucleotide primer may range from about 9 to about 13 nucleotides, with about 10 nucleotides being preferred. Arbitrary primer sequences cause the lengths of the amplified partial cDNAs produced to be variable, thus allowing different clones to be separated by using standard denaturing sequencing gel electrophoresis. PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times. The pattern of clones resulting from the reverse transcription and amplification of the mRNA of two different cell types is displayed via sequencing gel electrophoresis and compared. Differences in the two banding patterns indicate potentially differentially expressed genes.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ nontranscribed regulatory regions.

Commercially available capillary electrophoresis systems can be used to analyze the size or confirm the nucleotide sequence of PGR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer; ABI), and the entire process from loading of samples to computer analysis and electronic data display can be computer controlled. Capillary electrophoresis is especially preferable for the sequencing of small pieces of DNA which might be present in limited amounts in a particular sample.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis and/or RT-PCR. Upon corroboration, the differentially expressed genes may be further characterized, and may be identified as target and/or marker genes, as discussed, below.

Also, amplified sequences of differentially expressed genes obtained through, for example, differential display may be used to isolate full length clones of the corresponding gene. The full length coding portion of the gene may readily be isolated, without undue experimentation, by molecular biological techniques Well known in the art. For example, the isolated differentially expressed amplified fragment may be labeled and used to screen a cDNA library. Alternatively, the labeled fragment may be used to screen a genomic library.

An analysis of the tissue distribution of the mRNA produced by the identified genes may be conducted, utilizing standard techniques well known to those of skill in the art. Such techniques may include, for example, Northern analyses and RT-PCR. Such analyses provide information as to whether the identified genes are expressed in tissues expected to contribute to cancer. Such analyses may also provide quantitative information regarding steady state mRNA regulation, yielding data concerning which of the identified genes exhibits a high level of regulation in, preferably, tissues which may be expected to contribute to cancer.

Such analyses may also be performed on an isolated cell population of a particular cell type derived from a given tissue. Additionally, standard in situ hybridization techniques may be utilized to provide information regarding which cells within a given tissue express the identified gene. Such analyses may provide information regarding the biological function of an identified gene relative to cancer in instances wherein only a subset of the cells within the tissue is thought to be relevant to cancer.

### Identification of Polynucleotide Variants and Homologues or Splice Variants

Variants and homologues of the “CANCER GENE” polynucleotides described above also are “CANCER GENE” polynucleotides. Typically, homologous “CANCER GENE” polynucleotide sequences can be identified by hybridization of candidate polynucleotides to known “CANCER GENE” polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50 EC once, 30 minutes; then 2×SSC, room temperature twice, 10 minutes each homologous sequences can be identified which contain at most about 25-30% basepair mismatches. More preferably, homologous polynucleotide strands contain 15-25% basepair mismatches, even more preferably 5-15% basepair mismatches.

Species homologues of the “CANCER GENE” polynucleotides disclosed herein also can be identified by making suitable probes or primers and screening cDNA expression libraries from other species, such as mice, monkeys, or yeast. Human variants of “CANCER GENE” polynucleotides can be identified, for example, by screening human cDNA expression libraries. It is well known that the Tₘ of a double-stranded DNA decreases by 1-1.5° C. with every 1% decrease in homology [Bonner et al., 1973]. Variants of human “CANCER GENE” polynucleotides or “CANCER GENE” polynucleotides of other species can therefore be identified by hybridizing a putative homologous “CANCER GENE” polynucleotide with a polynucleotide having a nucleotide sequence of one of the genes of the Table 1 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to “CANCER GENE” polynucleotides or their complements following stringent hybridization and/or wash conditions also are “CANCER GENE” polynucleotides. Stringent wash conditions are well known and understood in the art and are disclosed, for example, in Sambrook et al., (6), Ausubel (7). Typically, for stringent hybridization conditions a combination of temperature and salt concentration should be chosen that is approximately 12 to 20° C. below the calculated Tₘ of the hybrid under study. The Tₘ of a hybrid between a “CANCER GENE” polynucleotide having a nucleotide sequence of one of the sequences of Table 1 or the complement thereof and a polynucleotide sequence which is at least about 50, preferably about 75, 90, 96, or 98% identical to one of those nucleotide sequences can be calculated, for example, using the equation below [Bolton and McCarthy, 1962];

Tₘ=81.5° C.−16.6(log₁₀[Na⁺])+0.41(% G+C)−0.63(% formamide)−600/l),

where 1=the length of the hybrid in basepairs.

Stringent wash conditions include, for example, 4×SSC at 65° C., or 50% formamide, 4×SSC at 28° C. or 0.5×SSC, 0.1% SDS alt 65° C. Highly stringent wash conditions include, for example, 0.2×SSC at 65° C.

### Polypeptides

“CANCER GENE” polypeptides according to the invention comprise a polypeptide of Table 1 or derivatives, fragments, analogues and homologues thereof. A “CANCER GENE” polypeptide of the invention therefore can be a portion, a full-length, or a fusion protein comprising all or a portion of a “CANCER GENE” polypeptide.

### Biologically Active Variants

“CANCER GENE” polypeptide variants which are biologically active, i.e., retain an “CANCER GENE” activity, can be also regarded as “CANCER GENE” polypeptides. Preferably, naturally or non-naturally occurring “CANCER GENE” polypeptide variants have amino acid sequences which are at least about 60, 65, or 70, preferably about 75, 80, 85, 90, 92, 94, 96, or 98% identical to any of the amino acid sequences of the polypeptides of encoded by the genes in Table 1 or the polypeptides encoded by any of the polynucleotides of Table 1 or a fragment thereof.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a “CANCER GENE” polypeptide can be found using computer programs well known in the art, such as DNASTAR software. Whether an amino acid change results in a biologically active “CANCER GENE” polypeptide can readily be determined by assaying for “CANCER GENE” activity, as described for example, in the specific Examples, below. Larger insertions or deletions can also be caused by alternative splicing. Protein domains can be inserted or deleted without altering the main activity of the protein.

### Detecting Expression and Gene Product

Although the presence of marker gene expression suggests that the “CANCER GENE” polynucleotide is also present, its presence and expression may need to be confirmed. For example, if a sequence encoding a “CANCER GENE” polypeptide is inserted within a marker gene sequence, transformed cells containing sequences which encode a “CANCER GENE” polypeptide can be identified by the absence of marker gene function. Alternatively, a marker gene can be placed in tandem with a sequence encoding a “CANCER GENE” polypeptide under the control of a single promoter. Expression of the marker gene in response to induction or selection usually indicates expression of the “CANCER GENE” polynucleotide.

Alternatively, host cells which contain a “CANCER GENE” polynucleotide and which express a “CANCER GENE” polypeptide can be identified by a variety of procedures known to those of skill in the art. These procedures include, but are not limited to, DNA-DNA or DNA-RNA hybridization and protein bioassay or immunoassay techniques which include membrane, solution, or chip-based technologies for the detection and/or quantification of polynucleotide or protein. For example, the presence of a polynucleotide sequence encoding a “CANCER GENE” polypeptide can be detected by DNA-DNA or DNA-RNA hybridization or amplification using probes or fragments or fragments of polynucleotides encoding a “CANCER GENE” polypeptide. Nucleic acid amplification-based assays involve the use of oligonucleotides selected from sequences encoding a “CANCER GENE” polypeptide to detect transformants which contain a “CANCER GENE” polynucleotide.

A variety of protocols for detecting and measuring the expression of a “CANCER GENE” polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site monoclonal-based immunoassay using monoclonal antibodies reactive to two non-interfering epitopes on a “CANCER GENE” polypeptide can be used, or a competitive binding assay can be employed. These and other assays are described in Hampton et al.

A wide variety of labels and conjugation techniques are known by those skilled in the art and can be used in various nucleic acid and amino acid assays. Means for producing labeled hybridization or PCR probes for detecting sequences related to polynucleotides encoding “CANCER GENE” polypeptides include oligo labeling, nick translation, end-labeling, or PCR amplification using a labeled nucleotide. Alternatively, sequences encoding a “CANCER GENE” polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radio-nuclides, enzymes, and fluorescent, chemiluminescent, or chromogenic agents, as well as substrates, cofactors, inhibitors, magnetic particles, and the like.

### Predictive, Diagnostic and Prognostic Assays

The present invention provides compositions, methods, and kits for determining the probability of successful application of a given mode of treatment in a subject having cancer in particular by detecting the disclosed biomarkers, i.e., the disclosed polynucleotide markers of Table 1.

In clinical applications, biological samples can be screened for the presence and/or absence of the biomarkers identified herein. Such samples are for example needle biopsy cores, surgical resection samples, or body fluids like serum, thin needle nipple aspirates and urine. For example, these methods include obtaining a biopsy, which is optionally fractionated by cryostat sectioning to enrich diseases cells to about 80% of the total cell population. In certain embodiments, polynucleotides extracted from these samples may be amplified using techniques well known in the art. The expression levels of selected markers detected would be compared with statistically valid groups of diseased and healthy samples.

In one embodiment the compositions, methods, and kits comprises determining whether a subject has an abnormal mRNA and/or protein level of the disclosed markers, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the levels of the disclosed biomarkers, protein or mRNA level, is determined and compared to the level of these markers in a healthy subject. An abnormal level of the biomarker polypeptide or mRNA levels is likely to be indicative of malignant neoplasia such as lung, ovarian, cervix, head and neck, stomach, pancreas, colon or breast cancer.

In another embodiment the compositions, methods, and kits comprises determining whether a subject has an abnormal DNA content of said genes or said genomic loci, such as by Southern blot analysis, dot blot analysis, Fluorescence or Colorimetric In Situ Hybridization, Comparative Genomic Hybridization or quantitative PCR. In general these assays comprise the usage of probes from representative genomic regions. The probes contain at least pans of said genomic regions or sequences complementary or analogous to said regions. In particular intra- or intergenic regions of said genes or genomic regions. The probes can consist of nucleotide sequences or sequences of analogous functions (e.g. PNAs, Morpholino oligomers) being able to bind to target regions by hybridization. In general genomic regions being altered in said patient samples are compared with unaffected control samples (normal tissue from the same or different patients, surrounding unaffected tissue, peripheral blood) or with genomic regions of the same sample that don't have said alterations and can therefore serve as internal controls. In a preferred embodiment regions located on the same chromosome are used. Alternatively, gonosomal regions and/or regions with defined varying amount in the sample are used. In one favored embodiment the DNA content, structure, composition or modification is compared that lie within distinct genomic regions. Especially favored are methods that detect the DNA content of said samples, where the amount of target regions are altered by amplification and or deletions. In another embodiment the target regions are analyzed for the presence of polymorphisms (e.g. Single Nucleotide Polymorphisms or mutations) that affect or predispose the cells in said samples with regard to clinical aspects, being of diagnostic, prognostic or therapeutic value. Preferably, the identification of sequence variations is used to define haplotypes that result in characteristic behavior of said samples with said clinical aspects.

### DNA Array Technology

In one embodiment, the present invention also provides a method wherein polynucleotide probes are immobilized an a DNA chip in an organized array. Oligonucleotides can be bound to a solid support by a variety of processes, including lithography. For example a chip can hold up to 410.000 oligonucleotides (GeneChip, Affymetrix). The present invention provides significant advantages over the available tests for malignant neoplasia, such as lung, ovarian, cervix, head and neck, stomach, pancreas, colon or breast cancer, because it increases the reliability of the test by providing an array of polynucleotide markers an a single chip.

The method includes obtaining a biological sample which can be a biopsy of an affected person, which is optionally fractionated by cryostat sectioning to enrich diseased cells to about 80% of the total cell population and the use of body fluids such as serum or urine, serum or cell containing liquids (e.g. derived from fine needle aspirates). The DNA or RNA is then extracted, amplified, and analyzed with a DNA chip to determine the presence of absence of the marker polynucleotide sequences. In one embodiment, the polynucleotide probes are spotted onto a substrate in a two-dimensional matrix or array, samples of polynucleotides can be labeled and then hybridized to the probes. Double-stranded polynucleotides, comprising the labeled sample polynucleotides bound to probe polynucleotides, can be detected once the unbound portion of the sample is washed away.

The probe polynucleotides can be spotted on substrates including glass, nitrocellulose, etc. The probes can be bound to the substrate by either covalent bonds or by non-specific interactions, such as hydrophobic interactions. The sample polynucleotides can be labeled using radioactive labels, fluorophores, chromophores, etc. Techniques for constructing arrays and methods of using these arrays are described in EP0 799 897; WO 97/29212; WO 97/27317; EP 0 785 280; WO 97/02357; U.S. Pat. No. 5,593,839; U.S. Pat. No. 5,578,832; EP 0 728 520; U.S. Pat. No. 5,599,695; EP 0 721 016; U.S. Pat. No. 5,556,752; WO 95/22058; and U.S. Pat. No. 5,631,734. Further, arrays can be used to examine differential expression of genes and can be used to determine gene function. For example, arrays of the instant polynucleotide sequences can be used to determine if any of the polynucleotide sequences are differentially expressed between normal cells and diseased cells, for example. High expression of a particular message in a diseased sample, which is not observed in a corresponding normal sample, can indicate a cancer specific protein.

Accordingly, in one aspect, the invention provides probes and primers that are specific to the polynucleotide sequences of Table 1.

In one embodiment, the composition, method, and kit comprise using a polynucleotide probe to determine the presence of malignant or cancer cells in particular in a tissue from a patient. Specifically, the method comprises;
1) providing a polynucleotide probe comprising a nucleotide sequence at least 12 nucleotides in length, preferably at least 15 nucleotides, more preferably, 25 nucleotides, and most preferably at least 40 nucleotides, and up to all or nearly all of the coding sequence which is complementary to a portion of the coding sequence of a polynucleotide selected from the polynucleotides of Table 1 or a sequence complementary thereto;
2) obtaining a tissue sample from a patient with malignant neoplasia;
3) providing a second tissue sample from a patient with no malignant neoplasia;
4) contacting the polynucleotide probe under stringent conditions with RNA of each of said first and second tissue samples (e.g., in a Northern blot or in situ hybridization assay); and
5) comparing (a) the amount of hybridization of the probe with RNA of the first tissue sample, with (b) the amount of hybridization of the probe with RNA of the second tissue sample;
   wherein a statistically significant difference in the amount of hybridization with the RNA of the first tissue sample as compared to the amount of hybridization with the RNA of the second tissue sample is indicative of malignant neoplasia and cancer in particular in the first tissue sample.

### Data Analysis Methods

Comparison of the expression levels of one or more “CANCER GENES” with reference expression levels, e.g., expression levels in diseased cells of cancer or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one “CANCER GENE” in a diseased cell. The values can be mRNA expression levels obtained from experiments, e.g., microarray analysis. The values can also be mRNA levels normalised relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data can be in the form of a table, such as an Excel table. The data can be alone, or it can be part of a larger database, e.g., comprising other expression profiles. For example, the expression profile data of the invention can be part of a public database. The computer readable form can be in a computer. In another embodiment, the invention provides a computer displaying the gene expression profile data.

In one embodiment, the invention provides a method for determining the similarity between the level of expression of one or more “CANCER GENES” in a first cell, e.g., a cell of a subject, and that in a second cell, comprising obtaining the level of expression of one or more “CANCER GENES” in a first cell and entering these values into a computer comprising a database including records comprising values corresponding to levels of expression of one or more “CANCER GENES” in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of “CANCER GENES” are entered into a computer system, comprising one or more databases with reference expression levels obtained from more than one cell. For example, the computer comprises expression data of diseased and normal cells. Instructions are provided to the computer, and the computer is capable of comparing the data entered with the data in the computer to determine whether the data entered is more similar to that of a normal cell or of a diseased cell.

In another embodiment, the computer comprises values of expression levels in cells of subjects at different stages of cancer, and the computer is capable of comparing expression data entered into the computer with the data stored, and produce results indicating to which of the expression profiles in the computer, the one entered is most similar, such as to determine the stage of cancer in the subject.

In yet another embodiment, the reference expression profiles in the computer are expression profiles from cells of cancer of one or more subjects, which cells are treated in vivo or in vitro with a drug used for therapy of cancer. Upon entering of expression data of a cell of a subject treated in vitro or in vivo with the drug, the computer is instructed to compare the data entered to the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In addition we challenged a classical PCA algorithm with the identification of the major components separating the samples and the two therapeutic outcomes.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes characteristic of cancer in a query cell with a database including records comprising reference expression or expression profile data of one or more reference cells and an annotation of the type of cell; and (ii) indicating to which cell the query cell is most similar based on similarities of expression profiles. The reference cells can be cells from subjects at different stages of cancer. The reference cells can also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides a method for selecting a therapy for a patient having cancer, the method comprising: (i) providing the level of expression of one or more genes characteristic of cancer in a diseased cell of the patient; (ii) providing a plurality of reference profiles, each associated with a therapy, wherein the subject expression profile and each reference profile has a plurality of values, each value representing the level of expression of a gene characteristic of cancer; and (iii) selecting the reference profile most similar to the subject expression profile, to thereby select a therapy for said patient. In a preferred embodiment step (iii) is performed by a computer. The most similar reference profile may be selected by weighing a comparison value of the plurality using a weight value associated with the corresponding expression data.

The relative abundance of an mRNA in two biological samples can be scored as a perturbation and its magnitude determined (i.e., the abundance is different in the two sources of mRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations can be used by a computer for calculating and expression comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of a stage of cancer or to a treatment for cancer.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory). Such hard disks are typically of 1 GB or greater storage capacity. Other external components include a user interface device, which can be a monitor, together with an inputting device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, IIL.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes characteristic of cancer. The database may contain one or more expression profiles of genes characteristic of cancer in different cells.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

### In Situ Hybridization

In one aspect, the method comprises in situ hybridization with a probe derived from a given marker polynucleotide, which sequence is selected from any of the polynucleotide sequences of the genes listed in Table 1 or a sequence complementary thereto. The method comprises contacting the labeled hybridization probe with a sample of a given type of tissue from a patient potentially having malignant neoplasia and cancer in particular as well as normal tissue from a person with no malignant neoplasia, and determining whether the probe labels tissue of the patient to a degree significantly different (e.g., by at least a factor of two, or at least a factor of five, or at least a factor of twenty, or at least a factor of fifty) than the degree to which normal tissue is labelled. In situ hybridization may be performed either to DNA in the nucleus of said cell in tissues or to the mRNA in the cytoplasm to stain for transcriptional activity.

### Polypeptide Detection

The subject invention further provides a method of determining whether a cell sample obtained from a subject possesses an abnormal amount of marker polypeptide which comprises (a) obtaining a cell sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide. Such marker polypeptides may be detected by immunohistochemical assays, dot-blot assays, ELISA and the like.

### Antibodies

Any type of antibody known in the art can be generated to bind specifically to an epitope of a “CANCER GENE” polypeptide. An antibody as used herein includes intact immunoglobulin molecules, as well as fragments thereof, such as Fab, F(ab)₂, and Fv, which are capable of binding an epitope of a “CANCER GENE” polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a “CANCER GENE” polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a “CANCER GENE” polypeptide provides a detection signal at least 5-, 10- or 20-fold higher than a detection signal provided with other proteins when used in an immunochemical assay. Preferably, antibodies which specifically bind to “CANCER GENE” polypeptides do not detect other proteins in immunochemical assays and can immunoprecipitate a “CANCER GENE” polypeptide from solution.

“CANCER GENE” polypeptides can be used to immunize a mammal, such as a mouse, rat, rabbit, guinea pig, monkey, or human, to produce polyclonal antibodies. If desired, a “CANCER GENE” polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a “CANCER GENE” polypeptide can be prepared using any technique which provides for the production of antibody molecules by continuous cell lines in culture. These techniques include, but are not limited to, the hybridoma technique, the human B cell hybridoma technique, and the EBV hybridoma technique [Kohler et al., 1985].

In addition, techniques developed for the production of chimeric antibodies, the splicing of mouse antibody genes to human antibody genes to obtain a molecule with appropriate antigen specificity and biological activity, can be used [Takeda et al., 1985]. Monoclonal and other antibodies also can be humanized to prevent a patient from mourning an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638 B. Antibodies which specifically bind to a “CANCER GENE” polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to “CANCER GENE” polypeptides. Antibodies with related specificity, but of distinct idiotypic composition, can be generated by chain shuffling from random combinatorial immunoglobulin libraries [Burton, 1991].

Single-chain antibodies also can be constructed using a DNA amplification method, such as PCR, using hybridoma cDNA as a template [Thirion et ah, 1996]. Single-chain antibodies can be mono- or bispecific, and can be bivalent or tetravalent. Construction of tetravalent, bispecific single-chain antibodies is taught, for example, in Coloma & Morrison. Construction of bivalent, bispecific single-chain antibodies is taught in Mallender & Voss.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology [Verhaar et al., 3995].

Antibodies which specifically bind to “CANCER GENE” polypeptides also can be produced by inducing in vivo production in the lymphocyte population or by screening immunoglobulin libraries or panels of highly specific binding reagents as disclosed in the literature [Orlandi et al, 1989].

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the antibodies described in WO 94/13804, also can be prepared.

Antibodies according to the invention can be purified by methods well known in the art. For example, antibodies can be affinity purified by passage over a column to which a “CANCER GENE” polypeptide is bound. The bound antibodies can then be eluted from the column using a buffer with a high salt concentration.

Immunoassays are commonly used to quantify the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarisation immunoassay (FPIA), fluorescence immunoassay (FLA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Other methods to quantify the level of a particular protein, or a protein fragment, or modified protein in a particular sample are based on flow-cytometric methods. Flow cytometry allows the identification of proteins on the cell surface as well as of intracellular proteins using fluorochrome labeled, protein specific antibodies or non-labeled antibodies in combination with fluorochrome labeled secondary antibodies. General techniques to be used in performing flow cytometric assays noted above are known to those of ordinary skill in the art. A special method based on the same principles is the microsphere-based flow cytometric. Microsphere beads are labeled with precise quantities of fluorescent dye and particular antibodies. Such techniques are provided by Luminex Inc. WO 97/14028. In another embodiment the level of a particular protein or a protein fragment, or modified protein in a particular sample may be determined by 2D gel-electrophoresis and/or mass spectrometry. Determination of protein nature, sequence, molecular mass as well charge can be achieved in one detection step. Mass spectrometry can be performed with methods known to those with skills in the art as MALDI, TOF, or combinations of these.

In another embodiment, the level of the encoded product, i.e., the product encoded by any of the polynucleotide sequences of the genes listed in Table 1 or a sequence complementary thereto, in a biological fluid (e.g., blood or urine) of a patient may be determined as a way of monitoring the level of expression of the marker polynucleotide sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of the disorder, e.g., plaque formation. The level of the marker polypeptide can be used predictively to evaluate whether a sample of cells contains cells which are, or are predisposed towards becoming, plaque associated cells. The observation of marker polypeptide level can be utilized in decisions regarding, e.g., the use of more stringent therapies.

As set out above, one aspect of the present invention relates to diagnostic assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

Of particular importance to the subject invention is the ability to quantify the level of marker polypeptide as determined by the number of cells associated with a normal or abnormal marker polypeptide level. The number of cells with a particular marker polypeptide phenotype may then be correlated with patient prognosis. In one embodiment of the invention, the marker polypeptide phenotype of the lesion is determined as a percentage of cells in a biopsy which are found to have abnormally high/low levels of the marker polypeptide. Such expression may be detected by immunohistochemical assays, dot-blot assays, ELISA and the like.

### Immunohistochemistry

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissues samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a Label for subsequent detection of binding, samples are incubated for a time Sufficient for formation of the immunocomplexes. Binding of the antibody is then detected by virtue of a Label conjugated to this antibody. Where the antibody is unlabeled, a second labeled antibody may be employed, e.g., which is specific for the isotype of the anti-marker polypeptide antibody. Examples of labels which may be employed include radionuclides, fluorescence, chemo luminescence, and enzymes.

Where enzymes are employed, the Substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular application where tissue samples are employed as it allows determination of the average amount of the marker polypeptide associated with a Single cell by correlating the amount of marker polypeptide in a cell-free extract produced from a predetermined number of cells.

In yet another embodiment, the invention contemplates using a panel of antibodies which are generated against the marker polypeptides of this invention, which polypeptides are encoded by any of the polynucleotide sequences of the genes from Table 1. Such a panel of antibodies may be used as a reliable diagnostic probe for cancer. The assay of the present invention comprises contacting a biopsy sample containing cells, e.g., macrophages, with a panel of antibodies to one or more of the encoded products to determine the presence or absence of the marker polypeptides.

The diagnostic methods of the subject invention may also be employed as follow-up to treatment, e.g., quantification of the level of marker polypeptides may be indicative of the effectiveness of current or previously employed therapies for malignant neoplasia and cancer in particular as well as the effect of these therapies upon patient prognosis.

The diagnostic assays described above can be adapted to be used as prognostic assays, as well. Such an application takes advantage of the sensitivity of the assays of the Invention to events which take place at characteristic stages in the progression of plaque generation in case of malignant neoplasia. For example, a given marker gene may be up- or down-regulated at a very early stage, perhaps before the cell is developing into a foam cell, while another marker gene may be characteristically up or down regulated only at a much later stage. Such a method could involve the steps of contacting the mRNA of a test cell with a polynucleotide probe derived from a given marker polynucleotide which is expressed at different characteristic levels in cancer tissue cells at different stages of malignant neoplasia progression, and determining the approximate amount of hybridization of the probe to the mRNA of the cell, such amount being an indication of the level of expression of the gene in the cell, and thus an indication of the stage of disease progression of the cell; alternatively, the assay can be carried out with an antibody specific for the gene product of the given marker polynucleotide, contacted with the proteins of the test cell. A battery of such tests will disclose not only the existence of a certain neoplastic lesion, but also will allow the clinician to select the mode of treatment roost appropriate for the disease, and to predict the likelihood of success of that treatment.

The methods of the invention can also be used to follow the clinical course of a given cancer predisposition. For example, the assay of the Invention can be applied to a blood sample from a patient; following treatment of the patient for CANCER, another blood sample is taken and the test repeated. Successful treatment will result in removal of demonstrate differential expression, characteristic of the cancer tissue cells, perhaps approaching or even surpassing normal levels. Modulation of Gene Expression

In another embodiment, test compounds which increase or decrease “CANCER GENE” expression are identified. A “CANCER GENE” polynucleotide is contacted with a test compound in an appropriate expression test system as described below or in a cell system, and the expression of an RNA or polypeptide product of the “CANCER GENE” polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of “CANCER GENE” mRNA or polypeptide expression in the cells can be determined by methods well known in the art for detecting mRNA or polypeptide. Either qualitative or quantitative methods can be used. The presence of polypeptide products of a “CANCER GENE” polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a “CANCER GENE” polypeptide.

Such screening can be carried out either in a cell-free assay system or in an intact cell. Any cell which expresses a “CANCER GENE” polynucleotide can be used in a cell-based assay system. A “CANCER GENE” polynucleotide can be naturally occurring in the cell or can be introduced using techniques such as those described above. Either a primary culture or an established cell line, such as CHO or human embryonic kidney 293 cells, can be used.

One strategy for identifying genes that are involved in cancer is to detect genes that are expressed differentially under conditions associated with the disease versus non-disease or in the context of therapy response conditions. The sub-sections below describe a number of experimental systems which can be used to detect such differentially expressed genes. In general, these experimental systems include at least one experimental condition in which subjects or samples are treated in a manner associated with cancer, in addition to at least one experimental control condition lacking such disease associated treatment or does not respond to such treatment. Differentially expressed genes are detected, as described below, by comparing the pattern of gene expression between the experimental and control conditions.

Once a particular gene has been identified through the use of one such experiment, its expression pattern may be further characterized by studying its expression in a different experiment and the findings may be validated by an independent technique. Such use of multiple experiments may be useful in distinguishing the roles and relative importance of particular genes in cancer and the treatment thereof. A combined approach, comparing gene expression pattern in cells derived from cancer patients to those of in vitro cell culture models can give substantial hints on the pathways involved in development and/or progression of cancer. It can also elucidate the role of such genes in the development of resistance or insensitivity to certain therapeutic agents (e.g. chemotherapeutic drugs).

Among the experiments which may be utilized for the identification of differentially expressed genes involved in malignant neoplasia and cancer in particular, are experiments designed 10 analyze those genes which are involved in signal transduction. Such experiments may serve to identify genes involved in the proliferation of cells.

Below are methods described for the identification of genes which are involved in cancer. Such represent genes which are differentially expressed in cancer conditions relative to their expression in normal, or non-cancer conditions or upon experimental manipulation based on clinical observations. Such differentially expressed genes represent “target” and/or “marker” genes. Methods for the further characterization of such differentially expressed genes, and for their identification as target and/or marker genes, are presented below.

Alternatively, a differentially expressed gene may have its expression modulated, i.e., quantitatively increased or decreased, in normal versus cancer states, or under control versus experimental conditions. The degree to which expression differs in normal versus cancer or control versus experimental states need only be large enough to be visualized via standard characterization techniques, such as, for example, the differential display technique described below. Other such standard characterization techniques by which expression differences may be visualized include but are not limited to quantitative RT-PCR and Northern analyses, which are well known to those of skill in the art.

In Addition to the experiments described above the following describes algorithms and statistical analyses which can be utilized for data evaluation and for the classification as well as response prediction for a so far not classified biological sample in the context of control samples. Predictive algorithms and equations described below have already shown their power to subdivide individual cancers.

### EXAMPLE 1

### Patient and Tumor Characteristics

The ethics committee of the University of Erlangen-Nuremberg approved the study protocols describing sample collection and gene profiling. Written consent was obtained from eligible patients, the research was conducted in accordance with the principles of the Declaration of Helsinki.

Biopsy samples from the primary tumor and one or more synchronous liver metastases were collected intraoperatively from 19 patients with UICC stage IV colorectal carcinoma at the time of resection of the primary tumor. Primary carcinoma was confirmed histologically. Histological confirmation was also obtained for synchronous liver metastasis. When metachronous liver metastasis was identified, histological confirmation was only pursued when imaging techniques (spiral computerized tomography (CT) of the abdomen or MRT of the liver) did not show clear results.

Patients received first-line chemotherapy, consisting of a weekly 1-2 hour infusion of folinic acid (500 mg m⁻²) followed by a 24-hour infusion of 5-fluorouracil (2600 mg m⁻²). One cycle comprised six weekly infusions followed by 2 weeks of rest. A total of 23 patients received additional biweekly oxaliplatin (85 mg m⁻²) and three patients also received irinotecan once per week (80 mg m⁻²). Treatment response was monitored every 8 weeks by spiral CT and antitumor activity was evaluated in accordance with WHO criteria. Median treatment duration was 7 months.

### Sample Preparation

Intraoperatively obtained biopsies were shock-frozen immediately (within one minute after removal) and stored at −80° C. The frozen tissues were cut into 8 μm sections using a cryostat and then stained with hematoxylin and eosin for histological examination. Laser capture microdissection (LCM) was performed immediately after staining and dehydration. Tumor areas of interest were selected with the help of an experienced pathologist (T.P.) and excised using a 0.6 mm laser beam (32 mW, 30 Hz, 0.8 sec pulse). Each sample yielded approximately 10.000 cells. Captured cells were dissolved in RLT buffer (RNeasy Mini Kit, Qiagen, Hilden, Germany) and RNA was extracted as described below

### RNA Extraction

Total RNA was isolated with the use of commercial kits (RNeasy-Mini Kit; Qiagen, Hilden, Germany) according to the manufacturer's instructions As part of this procedure, DNAse digestion (Qiagen, Hilden, Germany) was included before elution from the columns. The quantity and quality of the purified total RNA was measured with the use of the RNA Nano 6000 Assay Chip (Bioanalyzer 2100; Agilent Technologies, Palo Alto, Calif.).

### Gene Amplification

Each biopsy yielded up to 800 μg of total RNA. After several rounds of T7 promotor-based RNA amplification, each sample typically provided a final yield of 50-100 μg of amplified RNA (aRNA). We did reverse transcription with the MessageAmp aRNA Kit (Ambion, Huntingdon, United Kingdom) followed by in vitro transcription. During this later step a biotin label was added. The overall quality of the aRNA was assessed using the RNA Nano 6000 Assay Chip.

### Expression Profiling Utilizing DNA Microarrays

In brief, samples were hybridised to Affymetrix HG U133-A high-density oligonucleotide-based arrays (Affymetrix, Santa Clara/CA, USA) targeting 22,230 human genes and expressed sequence tags (EST). From each biopsy, 15 μg of either cRNA or aRNA was loaded onto an array following the recommended procedures for prehybridization, hybridization, washing and staining with streptavidin-phycoerythrin. The arrays were scanned on an Affymetrix GeneChip Scanner (Agilent, Palo Alto, Calif.). The fluorescence intensity was measured for each microarray and normalised to the average fluorescence intensity of the entire microarray.

General procedure: Expression profiling can be carried out using the Affymetrix Array Technology. By hybridization of mRNA to such a DNA-array or DNA-Chip, it is possible to identify the expression value of each transcripts due to signal intensity at certain position of the array. Usually these DNA-arrays are produced by spotting of cDNA, oligonucleotides or subcloned DNA fragments. In case of Affymetrix technology app. 400.000 individual oligonucleotide sequences were synthesized on the surface of a silicon wafer at distinct positions. The minimal length of oligomers is 12 nucleotides, preferable 25 nucleotides or full length of the questioned transcript. Expression profiling may also be carried out by hybridization to nylon or nitro-cellulose membrane bound DNA or oligonucleotides. Detection of signals derived from hybridization may be obtained by either colorimetric, fluorescent, electrochemical, electronic, optic or by radioactive readout. Detailed description of array construction have been mentioned above and in other patents cited. To determine the quantitative and qualitative changes in the gene expression of certain cancer specimens, RNA from tumor tissue extracted prior to any chemotherapy has to be compared among each other individually and/or to RNA extracted from benign tissue (e.g. epithelial tissue, or micro dissected ductal tissue) on the basis of expression profiles for the whole transcriptome. With minor modifications, the sample preparation protocol followed the Affymetrix GeneChip Expression Analysis Manual (Santa Clara, Calif.). Total RNA extraction and isolation from tumor or benign tissues, biopsies, cell isolates or cell containing body fluids can be performed by using TRIzol (Life Technologies, Rockville., MD) and Oligotex mRNA Midi kit (Qiagen, Hilden, Germany), and an ethanol precipitation step should be carried out to bring the concentration to 1 mg/ml. Using 5-10 mg of mRNA to create double stranded cDNA by the Superscript system (Life Technologies). First strand cDNA synthesis was primed with a T7-(dT24) oligonucleotide. The cDNA can be extracted with phenol/chloroform and precipitated with ethanol to a final concentration of 1 mg/ml. From the generated cDNA, cRNA can be synthesized using Enzo's (Enzo Diagnostics Inc., Farmingdale, N.Y.) in vitro Transcription Kit. Within the same step the cRNA can be labeled with biotin nucleotides Bio-11-CTP and Bio-16-UTP (Enzo Diagnostics Inc., Farmingdale, N.Y.). After labeling and cleanup (Qiagen, Hilden (Germany) the cRNA then should be fragmented in an appropriated fragmentation buffer (e.g., 40 mM Tris-Acetate, pH 8,1, 100 mM KOAc, 30 mM MgOAc, for 35 minutes at 94° C.). As per the Affymetrix protocol, fragmented cRNA should be hybridized on the HG_U133 arrays (as used herein), comprising app. 40.000 probed transcripts each, for 24 hours at 60 rpm in a 45° C. hybridization oven. After Hybridization step the chip surfaces have to be washed and stained with streptavidin phycoerythrin (SAPE; Molecular Probes, Eugene, Oreg.) in Affymetrix fluidics stations. To amplify staining, a second labeling step can be introduced, which is recommended but not compulsive. Here one should add SAPE solution twice with an antistreptavidin biotinylated antibody. Hybridization to the probe arrays may be detected by fluorometric scanning (Hewlett Packard Gene Array Scanner; Hewlett Packard Corporation, Palo Alto, Calif.). 1

After hybridization and scanning, the microarray images can be analyzed for quality control, looking for major chip defects or abnormalities in hybridization signal. Therefor either Affymetrix GeneChip MAS 5.0 Software or other microarray image analysis software can be utilized. Primary data analysis should be carried out by software provided by the manufacturer. In case of the genes analyses in one embodiment of this invention the primary data have been analyzed by further bioinformatic tools and additional filter criteria as described in examples.

Data Analysis from Expression Profiling Experiments

In brief, the raw, unnormalized data-sets were analyzed by MicroArray Suite (Affymetrix) for normalization and expression estimation. Signal intensities, detection calls, sample comparison by statistical analysis (t-Test, Welch, Kolmogorov-Smimov, Wilcoxon), hierarchical clustering, summary statistical analysis (principal component analysis, MOPS analysis, Fishers Exact Test), gene ranking, classification analysis and cross validation (K nearest neighbors, support vector machine, Sparse linear Discriminant Analysis, Fisher linear Discriminant Analysis), were determined using the GeneChip 5.0 software (Affymetrix) and Expressionist™ software (Genedata). Kaplan Meier Statistics was performed by using GraphPad Prism 4 ® (GraphPad Software Inc.). Significance levels of microarray results for primary colorectal cancer vs. synchronous liver metastases were calculated using the Welch, Kolmogorov-Smirnov, Wilcoxon and t-Test. A p value of < 0.05 was regarded as significant

According to Affymetrix measurement technique (Affymetrix GeneChip Expression Analysis Manual, Santa Clara, Calif.) a single gene expression measurement on one chip yields the average difference value and the absolute call. Each chip contains 16-20 oligonucleotide probe pairs per gene or cDNA clone. These probe pairs include perfectly matched sets and mismatched sets, both of which are necessary for the calculation of the average difference, or expression value, a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. The average difference is a numeric value supposed to represent the expression value of that gene. The absolute call can take the values ‘A’ (absent), ‘M’ (marginal), or ‘P’ (present) and denotes the quality of a single hybridization. We used both the quantitative information given by the average difference and the qualitative information given by the absolute call to identify the genes which are differentially expressed in biological samples from individuals with cancer versus biological samples from the normal population. With other algorithms than the Affymetrix one we have obtained different numerical values representing the same expression values and expression differences upon comparison.

The differential expression E in one of the cancer groups compared to the normal population is calculated as follows. Given n average difference values d1, d2, dn in the cancer population and m average difference values c1, c2, . . . , cm in the population of normal individuals, it is computed by the equation;

$$E ≡ exp ( \frac{1}{m} {\sum }_{i = 1}^{m} ln ( {c}_{i} ) - \frac{1}{n} {\sum }_{i = 1}^{n} ln ( {d}_{i} ) ) \qquad ( Equation 1 )$$

If dj<50 or ci<50 for one or more values of i and j, these particular values ci and/or dj are set to an “artificial” expression value of 50. These particular computation of E allows for a correct comparison to TaqMan results. A gene is called up-regulated in cancer of good or bad outcome, if E>=average change factor if the number of absolute calls equal to ‘P’ in the cancer population is greater than n/2.

Table 1 depicts the genes, whose varying gene expression levels can be used to predict clinical outcome of cancer patients. Gene Symbol, gene description, ref, sewunce, Unigene ID and OMIM number are displayed.

TABLE 1
Genes differentially expressed and capable of predicting therapeutic success.

| Gene Symbol | Gene Description | Ref. Sequences | dna_seq | prot_seq | Unigene ID | OMIM |
|---|---|---|---|---|---|---|
| HOXA6 | homeobox protein A6 | NM_024014 | 1 | 40 | Hs.248073 | 142851 |
| HOXA7 | homeobox protein A7 | NM_006896 | 2 | 41 | Hs.70954 | 142950 |
| HOXA9 | homeobox protein A9 isoform b | NM_002142 | 3 | 42 | Hs.127428 | 142956 |
| HOXA10 | homeobox protein A10 isoform a | NM_018951 | 4 | 43 | Hs.110637 | 142957 |
| HOXB2 | homeo box B2 | NM_002145 | 5 | 44 | Hs.2733 | 142967 |
| HOXB6 | homeo box B6 isoform 1 | NM_018952 | 6 | 45 | Hs.98428 | 142951 |
| HOXC4 | homeo box C4 | NM_014620 | 7 | 46 | Hs.50895 | 142974 |
| HOXC10 | homeo box C10 | NM_017409 | 8 | 47 | Hs.44276 | 605960 |
| HOXD4 | homeo box D4 | NM_014621 | 9 | 48 | Hs.278255 | 142981 |
| HOXD9 | homeo box D9 | NM_014213 | 10 | 49 | Hs.236846 | 142982 |
| HOXD11 | homeo box D11 | NM_021192 | 11 | 50 | Hs.421136 | 142988 |
| HOXD12 | homeo box D12 | NM_021193 | 12 | 51 | Hs.263958 | 142988 |
| MMP1 | matrix metalloproteinase 1 preproprotein | NM_002421 | 13 | 52 | Hs.83τ69 | 120353 |
| MMP2 | matrix metalloproteinase 2 preproprotein | NM_004530 | 14 | 53 | Hs.11τ301 | 120360 |
| MMP3 | matrix metalloproteinase 3 preproprotein | NM_002422 | 15 | 54 | Hs.83326 | 185250 |
| MMP7 | matrix metalloproteinase 7 preproprotein | NM_002423 | 16 | 55 | Hs.2256 | 178990 |
| MMP9 | matrix metalloproteinase 9 preproprotein | NM_004994 | 17 | 56 | Hs.151738 | 120361 |
| MMP12 | matrix metalloproteinase 12 preproprotein | NM_002426 | 18 | 57 | Hs.1695 | 601046 |
| TIMP1 | tissue inhibitor of metalloproteinase 1 precursor | NM_003254 | 19 | 58 | Hs.5831 | 305370 |
| TIMP2 | tissue inhibitor of metalloproteinase 2 precursor | NM_003255 | 20 | 59 | Hs.325495 | 188825 |
| ME1 | cytosolic malic enzyme 1 | MM_002395 | 21 | 60 | Hs.14732 | 154250 |
| ME2 | malic enzyme 2, NAD(+)-dependent, mitochondrial | NM_002396 | 22 | 61 | Hs.75342 | 154270 |
| KDR | kinase insert domain receptor (a type III receptor tyrosine kinase) | NM_002253 | 23 | 62 | Hs.12337 | 191306 |
| FLT3 | fms-related tyrosine kinase 3 | NM_004119 | 24 | 63 | Hs.385 | 136351 |
| FLT4 | fms-related tyrosine kinase 4 | NM_002020 | 25 | 64 | Hs.74049 | 136352 |
| VEGF | vascular endothelial growth factor | NM_003375 | 26 | 65 | Hs.73793 | 192240 |
| VEGFB | vascular endothelial growth factor B | NM_003377 | 27 | 66 | Hs.78781 | 601398 |
| VEGFC | vascular endothelial growth factor C preproprotein | NM_005429 | 28 | 67 | Hs.79141 | 601528 |
| EGFR | epidermal growth factor receptor 1 | NM_005226 | 29 | 68 | Hs.77432 | 131550 |
| ERBB2 | v-erb-b2 erythroblastic leukemia viral oncogene homolog 2, | NM_004448 | 30 | 69 | Hs.323910 | 164870 |
| ERBB3 | v-erb-b2 erythroblastic leukemia viral oncogene homolog 3 | NM_001982 | 31 | 70 | Hs.199067 | 190151 |
| ERBB4 | v-erb-a erythroblastic leukemia viral oncogene homolog 4 | NM_005235 | 32 | 71 | Hs.1939 | 600543 |
| PPARG | peroxisome proliferative activated receptor gamma isoform 1 | NM_005037 | 33 | 72 | Hs.100724 | 601487 |
| AKR1C1 | aido-keto reductase family 1, member C1 | NM_001353 | 34 | 73 | Hs.306098 | 600449 |
| PLCB4 | Homo sapiens phospholipase C, beta 4 (PLCB4) | NM_182797 | 35 | 74 | Hs.293006 | 600810 |
| MAP3KS | MAP/ERK kinase kinase 5 | NM_005923 | 36 | 75 | Hs.151988 | 602448 |
| SPON1 | spondin 1, (f-spondin) extracellular matrix protein | NM_006108 | 37 | 76 | Hs.5378 | 604988 |
| ORM1 | orosomucoid 1 precursor | NM_000607 | 38 | 77 | Hs.572 | 138600 |
| APCS | serum amyloid P component precursor | NM_001839 | 39 | 78 | Hs.1957 | 104770 |
| FIGF/VE [symbol] | vascular endothelial growth factor D preproprotein | NM_004469 | 40 | 79 | Hs.11392 | 300091 |
| HOXA4 | homeobox protein A4 | NM_002141 | 41 | 80 | Hs.77637 | 142953 |
| HOXA5 | homeobox protein A5 | NM_019102 | 42 | 81 | Hs.37034 | 142952 |
| HOXB5 | homeo box B5 | NM_002147 | 43 | 82 | Hs.22554 | 142960 |
| HOXC6 | homeo box C6 isoform 1 | NM_004503 | 44 | 83 | Hs.820 | 142972 |
| MMP10 | matrix metalloproteinase 10 preproprotein | NM_002425 | 45 | 84 | Hs.2258 | 185260 |
| TIMP3 | tissue inhibitor of metalloproteinase 3 precursor | NM_000362 | 46 | 85 | Hs.245188 | 188826 |
| HOXA9 | homeobox protein A9 isoform a | NM_152739 | 47 | 86 | Hs.127428 | 142956 |
| HOXA10 | homeobox protein A10 isoform b | NM_153715 | 48 | 87 | Hs.110637 | 142957 |

[symbol]

indicates data missing or illegible when filed

A gene is called up-regulated in cancer of good or bad outcome, if E>=average change factor given in Table 2 and if the number of absolute calls equal to ‘P’ in the cancer population is greater than n/2. The average change factor of candidate gene expression in primary tumor and/or metastatic lesion is depicted as ratio of medians in Table 2 for those patients suffering a tumor responding (sample group 1) or non responding to 5′ FU based anti-cancer regimen (sample group 2).

Table 2 A depicts the genes the ratio of medians when comparing gene expression levels of the depicted candidate genes between responding and non-responding tumors. The respective analysis did contain primary tumors and metastatic lesions. Therefore some genes whose discriminative power is more prominent in primary tumors (e.g. MMP family, EGFR family) do display relatively low ratio of medians (fresh tissue analysis by Affymetrix profiling). Gene Symbol, biological function, molecular function and ratio of medians are displayed.

TABLE 2 A
Ratio of medians of candicate genes when comparing responding and non-responding tumors (containing both primary tumors and metastasis).

| Gene Symbol | Biological Function [C] | Ref. Sequence | Ratio of Medians |
|---|---|---|---|
| ORM1 | acute-phase response(experimental evidence) inflammatory response(experimental evidence) | NM_000607 | 14.14 |
| SPON1 | inhibition of APP processing thereby impairing APP signalling | NM_006108 | 9.41 |
| PLOB4 | regulation of phosphoinositol signalling | — | 9.36 |
| MMP3 | proteolysis and peptidolysis(experimental evidence) | NM_002422 | 4.82 |
| APCS | pathogenesis(not recorded) protein folding, DNA packaging, protein complex assembly | NM_001639 | 4.06 |
| HOXD11 | embryogenesis and morphogenesis(experimental evidence) | NM_021192 | 3.82 |
| HOXA9 | developmental processes(predicted/computed) oncogenesis(predicted/computed) | NM_002142 | 3.79 |
| PPARG | signal transduction, lipid metabolism, nutritional response pathway, energy pathways(predicted) | NM_005037 | 3.69 |
| HOXD9 | embryogenesis and morphogenesis(experimental evidence) | NM_014213 | 3.11 |
| HOXA10 | oncogenesis, developmental processes, spermatogenesis(predicted/computed) | NM_018951 | 2.67 |
| HOXD4 | embryogenesis and morphogenesis(experimental evidence) | NM_014621 | 2.64 |
| AKR1C1 | xenobiotic metabolism(predicted/computed) | NM_001353 | 2.49 |
| MMP1 | proteolysis and peptidolysis(predicted/computed) | NM_002421 | 2.38 |
| TIMP2 | metalloprotease inhibitor | NM_003255 | 2.21 |
| MAP3K5 | induction of apoptosis by extracellular signals, MAPKKK cascade, activation of JUN kinase | NM_005923 | 2.13 |
| ME1 | malate metabolism(not recorded), carbohydrate metabolism(not recorded) | NM_002395 | 2.12 |
| ERBB2 | transmembrane receptor protein tyrosine kinase signalling pathway(experimental evidence) | NM_004446 | 1.88 |
| HOXA7 | embryogenesis and morphogenesis | NM_006896 | 1.65 |
| FLT3 | positive control of cell proliferation, receptor protein tyrosine kinase signalling pathway | NM_004119 | 1.52 |
| VEGF | positive control of cell proliferation, stress response, homophilic cell adhesion, signal transduction | NM_003376 | 1.46 |
| TIMP1 | positive control of cell proliferation (experimental evidence) | NM_003254 | 1.45 |
| VEGFC | signal transduction, positive control of cell proliferation. substrate-bound cell migration | NM_005429 | 1.44 |
| MMP2 | proteolysis and peptidolysis | NM_004530 | 1.43 |
| MMP9 | proteolysis and peptidolysis(experimental evidence) | NM_004994 | 1.38 |
| ERBB4 | cell proliferation, oncogenesis, developmental processes | NM_005235 | 1.35 |
| FLT4 | transmembrane receptor protein tyrosine kinase signalling pathway | NM_002020 | 1.33 |
| MMP12 | proteolysis and peptidolysis(experimental evidence) cell motility(not recorded) | NM_002425 | 1.25 |
| KDR | transmembrane receptor protein tyrosine kinase signalling pathway(experimental evidence) | NM_002255 | 1.25 |
| MMP7 | proteolysis and peptidolysis(predicted/computed) | NM_002423 | 1.21 |
| ERBB3 | protein phosphorylation(experimental evidence) | NM_001982 | 1.14 |
| ME2 | pyruvate metabolism(not recorded) | NM_002396 | 1.13 |
| EGFR | cell proliferation, cell shape and cell size control, EGF receptor signalling pathway | NM_005228 | 1.05 |
| VEGFB | signal transduction(experimental evidence) positive control of cell proliferation | NM_003377 | 1.04 |

Table 2 B depicts the genes the ratio of medians when comparing gene expression levels of the depicted candidate genes in primary tumors, whose corresponding metastatic lesions did or did not respond to anti-cancer regimen. The respective analysis did contain only primary tumors. Therefore the discriminative power of some genes as determined by fresh tissue analysis by Affymetrix profiling is more prominent in primary tumors than in the corresponding metastatic lesions (e.g. compare MMP family members in table 2A and table 2B). Gene Symbol, biological function, and ratio of medians are displayed.

TABLE 2 B
Ratio of medians of candicate genes when comparing responding
and non-responding tumors (containing only primary tumors).

| Gene Symbol | Molecular Function | Ratio of Medians |
|---|---|---|
| PLCB4 | phospholipase C | 8.07 |
| SPON1 | APP binding | 6.96 |
| MMP12 | macrophage elastase; zinc binding | 6.22 |
| MMP1 | zinc binding: collagenase | 5.57 |
| PPARG | ligand-dependent nuclear receptor: transcription factor: recep [symbol] | 4.74 |
| MMP3 | stromelysin | 3.75 |
| HOXD12 | transcription factor | 3.59 |
| VEGFC | vascular endothelial growth factor receptor ligand | 3.38 |
| HOXA10 | transcription factor | 3.09 |
| MMP9 | zinc binding: collagenase | 2.92 |
| HOXA9 | | 2.86 |
| HOXC10 | RNA polymerase II transcription factor | 2.77 |
| HOXA5 | transcription factor | 2.43 |
| ERBB2 | Neu/ErbB- receptor: receptor signalling protein tyrosine kinas [symbol] | 2.28 |
| TIMP2 | metalloprotease inhibitor | 2.25 |
| KDR | vascular endothelial growth factor receptor | 2.20 |
| MMP2 | gelatinase A: zinc binding | 2.19 |
| HOXB2 | transcription factor | 2.13 |
| MMP10 | metalloendopeptidase: zinc binding | 2.12 |
| ORM1 | acute-phase response protein: plasma glycoprotein | 2.08 |
| HOXC6 | transcription co-repressor | 2.01 |
| HOXD9 | RNA polymerase II transcription factor | 1.95 |
| HOXC4 | transcription factor | 1.87 |
| ME1 | malate dehydrogenase: electron transporter: | 1.82 |
| HOXB6 | transcription factor | 1.75 |
| TIMP1 | metalloprotease inhibitor | 1.72 |
| MAP3K5 | MAP kinase kinase kinase | 1.71 |
| HOXA4 | transcription factor | 1.70 |
| VEGF | vascular endothelial growth factor receptor ligand | 1.58 |
| FIGF | ligand: platelet-derived growth factor receptor ligand | 1.50 |
| MMP7 | metalloendopeptidase | 1.44 |
| VEGFB | vascular endothelial growth factor receptor ligand | 1.43 |
| HOXD11 | transcription factor | 1.39 |
| HOXB5 | transcription factor | 1.33 |
| APCS | amyloid protein: chaperone: plasma glycoprotein: acute-pha [symbol] | 1.27 |
| HOXB5 | transcription factor | 1.27 |
| HOXB6 | transcription factor | 1.25 |
| HOXA7 | transcription factor | 1.24 |
| EGFR | epidermal growth factor receptor | 1.23 |
| HOXA6 | transcription factor | 1.21 |
| FLT3 | vascular endothelial growth factor receptor | 1.19 |
| ERBB4 | transmembrane receptor protein tyrosine kinase | 1.15 |
| FLT4 | transmembrane receptor protein tyrosine kinase: vascular en [symbol] | 1.13 |
| ERBB3 | transmembrane receptor protein tyrosine kinase: epidermal gr [symbol] | 1.03 |
| AKR1C1 | enzyme: ligand binding or carrier: bile acid transporter: aldo [symbol] | 1.02 |
| HOXD4 | transcription factor | 1.01 |
| ME2 | malate dehydrogenase: electron transporter | 1.01 |

[symbol]

indicates data missing or illegible when filed

Table 2 C depicts the genes the ratio of medians when comparing gene expression levels of the depicted candidate genes in primary tumors, on basis of the overall survival of the patients (OAS>16 month vs<10 month). The respective analysis did contain only primary tumors. Therefore the discriminative power of some genes as determined by fresh tissue analysis by Affymetrix profiting is more prominent in primary tumors than in the corresponding metastatic lesions (e.g. compare MMP family members in table 2A and table 2B). Gene Symbol, molecular function and ratio of medians are displayed.

TABLE 2 C
Ratio of medians of candicate genes expressed in
primary tumors when comparing overall survival of
patients (OAS >16 month vs <10 month).

| Gene Symbol | Ratio of Medians |
|---|---|
| PLCB4 | 7.83 |
| SPON1 | 4.52 |
| HOXA9 | 3.62 |
| HOXD12 | 3.59 |
| HOXA10 | 2.66 |
| HOXC10 | 2.48 |
| PPARG | 2.46 |
| ERBB4 | 2.42 |
| HOXB2 | 2.31 |
| MMP2 | 2.29 |
| MMP9 | 2.21 |
| HOXB6 | 2.20 |
| TIMP2 | 2.20 |
| HOXC4 | 2.18 |
| ME1 | 2.11 |
| MMP10 | 2.06 |
| MMP3 | 2.01 |
| MMP1 | 2.00 |
| HOXA5 | 2.00 |
| EGFR | 1.88 |
| AKR1C1 | 1.88 |
| VEGFC | 1.87 |
| HOXD11 | 1.76 |
| ORM1 | 1.78 |
| TIMP1 | 1.70 |
| HOXA4 | 1.56 |
| FIGF | 1.55 |
| MAP3K5 | 1.55 |
| HOXB5 | 1.43 |
| HOXC6 | 1.39 |
| VEGFB | 1.38 |
| HOXB5 | 1.27 |
| VEGF | 1.27 |
| KDR | 1.22 |
| HOXA6 | 1.22 |
| HOXA7 | 1.21 |
| APCS | 1.19 |
| HOXB5 | 1.19 |
| ME2 | 1.17 |
| FLT3 | 1.17 |
| MMP12 | 1.17 |
| ERBB3 | 1.16 |
| ERBB2 | 1.15 |
| HOXD9 | 1.10 |
| HOXD4 | 1.06 |
| FLT4 | 1.05 |
| MMP7 | 1.04 |

Fold changes greater than 1 refers to a difference in gene expression between the sample cohorts. This regulation factors are median values and may differ individually, here the combined profiles genes listed in Table 1 in a cluster analysis or a principle component analysis (PCA) will indicate the classification group for such sample (see below for representative PCA with multiple genes and multiple classes). By a PCA one will identify the major components (Eigengenes or Eigenvectors) which do discriminate the samples analyzed.

### Data Filtering:

Raw data of the qRT-PCR were normalized to one or combinations of the housekeeping genes RPL37A, GAPDH, RPL9 and CD63 by using the comparative ΔΔCT method, known to those with skills in the art. In brief, all experiments were normalized by adjusting the respective housekeeping gene to a CT value of 25. “Copy numbers” of each gene were then calculated by 2^{(40-gene×mormalized CT value)}. Raw data of gene array analysis were acquired using Microsuite 5.0 software of Affymetrix and normalized following a standard practice of scaling the average of all gene signal intensities to a common arbitrary value. 59 Genes corresponding to Affymetrix controls (housekeeping genes, etc.) were removed from the analysis. The only exception has been done for the genes for GAPDH and Beta-actin, which expression levels were used for the normalization purposes. One hundred genes, which expression levels are routinely used in order to normalized between HG-U133A and HG-U133B GeneChips, were also removed from the analysis. Genes with potentially high levels of noise (81 probe sets), which is observed for genes with low absolute expression values (genes, which expression levels did not achieve 30 RLU (TGT=100) through all experiments), were removed from the data set. The remaining genes were preprocessed to eliminate the genes (3196 probe sets) whose signal intensities were not significantly different from their background levels and thus labeled as “Absent” by Affymetrix MicroSuite 5.0 in all experiments. We eliminated genes that were not present in at least 10% of samples (3841 probe sets). Data for remaining 15,006 probe sets were subsequently analysed by statistical methods.

### Statistical Analysis;

In order to optimize prediction of outcome one may use this class from the training cohort and run multiple statistical rests, suitable for group comparison including nonparametric Wilcoxon rank sum test, two-sample independent Students' t-test, Welch test, Kolmogorov-Smirnov test (for variance), and SUM-Rank test. We could identify such genes with a differential expression in the responding group vs. the non responding group and a significance level (p-value) below 0.05 as exemplified in Table 3. Hereby we verified statistical significance of the selected candidate genes displayed in Table 1.

Table 3A depicts the results of statistical analysis of candidate genes (p-values of different statistical methods), when comparing responding and non responding tumors (fresh tissue analysis by Affymetrix profiling). Gene Symbol, Ref. Sequence, Unigene ID, OMIM, T-test, Welch test, Kohnogorov-Smirnov and SUM-Rank test are displayed.

TABLE 3A
Statistical analysis of genes discriminating between responding and non-responding tumors (primary tumors and metastasis).

| Gene Symbol | Ref. Sequenc [symbol] | Unigene ID | OMIM | T-Test | Welch | Kolmogorov-S [symbol] | Wilcoxon | Rank Sum |
|---|---|---|---|---|---|---|---|---|
| HOXD9 | NM_014213 | 236646 | 142982 | 1.799E−06 | 7.082E−06 | 8.227E−05 | 8.227E−05 | 1 |
| HOXD11 | NM_021192 | 421136 | 142986 | 1.486E−06 | 2.036E−05 | 8.227E−05 | 8.227E−05 | 2 |
| ORM1 | NM_000607 | 572 | 138600 | 1.514E−05 | 1.789E−05 | 8.227E−05 | 8.227E−05 | 3 |
| PLCB4 | — | 283006 | — | 0.0003416 | 0.001089 | 8.227E−05 | 8.227E−05 | 4 |
| HOXD4 | NM_014621 | 278255 | 142981 | 0.000262 | 0.0003595 | 0.004278 | 0.0005759 | 5 |
| APCS | NM_001639 | 1957 | 104770 | 0.001297 | 0.001223 | 0.004278 | 0.003702 | 6 |
| PPARG | NM_005037 | 100724 | 601487 | 0.001403 | 0.001286 | 0.01119 | 0.002468 | 7 |
| AKR1C1 | NM_001353 | 306098 | 600449 | 0.001878 | 0.001973 | 0.008309 | 0.002468 | 8 |
| SPON1 | NM_006108 | 5378 | 604989 | 0.004005 | 0.004605 | 0.02024 | 0.005512 | 9 |
| TIMP2 | NM_003255 | 325495 | 188625 | 0.01158 | 0.01481 | 0.02024 | 0.01522 | 10 |
| MAP3K5 | NM_005923 | 151988 | 602448 | 0.006586 | 0.007552 | 0.04689 | 0.01522 | 11 |
| HOXA9 | NM_002142 | 127428 | 142956 | 0.05402 | 0.05343 | 0.008309 | 0.01522 | 12 |
| HOXA7 | NM_006896 | 70954 | 142950 | 0.0299 | 0.02805 | 0.03357 | 0.01522 | 13 |

[symbol]

indicates data missing or illegible when filed

Table 3B depicts the results of statistical analysis of candidate genes (p-values of different statistical methods) expressed in primary tumors, whose synchronous metastasis did respond or did not respond to 5′ FU based regimen (fresh tissue analysis by Affymetrix profiling). Gene Symbol, Ref. Sequence, Unigene ID, OMIM, T-test and SUM-Rank test are displayed.

TABLE 3B
Statistical analysis of genes discriminating between responding
and non-responding tumors (primary tumors and metastasis).

| Gene Symbol | Ref. Sequences | Unigene ID | OMIM | T-Test | Welch | Rank Sum |
|---|---|---|---|---|---|---|
| MMP12 | NM_002426 | 1695 | 601046 | 0.001187 | 0.01453 | 1 |
| PPARG | NM_005037 | 100724 | 601487 | 0.008442 | 0.009102 | 2 |
| VEGFC | NM_005429 | 79141 | 601528 | 0.002556 | 0.02242 | 3 |
| SPON1 | NM_006108 | 5376 | 604989 | 0.008706 | 0.01599 | 4 |
| HOXA9 | NM_002142 | 127428 | 142956 | 0.003921 | 0.042010002 | 5 |
| MMP2 | NM_004530 | 111301 | 120360 | 0.01136 | 0.02139 | 6 |
| MMP1 | NM_002421 | 63169 | 120353 | 0.028279999 | 0.0222 | 7 |
| HOXD12 | NM_021193 | 283958 | 142988 | 0.01278 | 0.051890001 | 8 |
| HOXA10 | NM_018951 | 110637 | 142957 | 0.01555 | 0.068510003 | 9 |
| MMP3 | NM_002422 | 83326 | 185250 | 0.046709999 | 0.051109999 | 10 |
| APCS | NM_001639 | 1957 | 104770 | 0.059319999 | 0.047699999 | 11 |
| HOXD11 | NM_021192 | 421136 | 142986 | 0.061069999 | 0.068570003 | 12 |
| TIMP2 | NM_003255 | 325495 | 188825 | 0.042350002 | 0.128700003 | 13 |
| MMP10 | NM_002425 | 2258 | 185260 | 0.05926 | 0.152899995 | 14 |

Table 3C depicts the results of statistical analysis of candidate genes (p-values of different statistical methods) expressed in primary tumors, on basis of the overall survival of the patients (OAS>16 month vs<10 month; comparison of fresh tissue analysis by Affymetrix profiling). Gene Symbol, Ref. Sequence, Unigene ID, OMIM, T-test and SUM-Rank test are displayed.

TABLE 3C
Statistical analysis of genes discriminating between long term and short term survivors
of patients suffering mCRC and receiving 5′ FU based chemotherapy based on fresh
tissue expression profiling by Affymetrix genechips of primary tumors.

| Gene Symbol | T-Test | Welch | Kolmogorov-Smimc [symbol] | Wilcoxon | Rank Sum |
|---|---|---|---|---|---|
| MMP3 | 0.018750001 | 0.01991 | 0.015869999 | 0.015869999 | 1 |
| ME1 | 0.009264 | 0.03334 | 0.015869999 | 0.015869999 | 2 |
| PLCB4 | 0.004658 | 0.003926 | 0.079369998 | 0.031750001 | 3 |
| HOXB2 | 0.024870001 | 0.02263 | 0.079369998 | 0.031750001 | 4 |
| FIGF | 0.038660001 | 0.081589997 | 0.015889999 | 0.015869999 | 5 |
| HOXA6 | 0.036660001 | 0.03101 | 0.079369999 | 0.031750001 | 6 |
| MMP2 | 0.031720001 | 0.029270001 | 0.079369999 | 0.063490003 | 7 |
| AKR1C1 | 0.080710001 | 0.077639997 | 0.079369999 | 0.031750001 | 8 |
| HOXD12 | 0.029759999 | 0.02581 | 0.142900005 | 0.063490003 | 9 |
| HOXC4 | 0.052850001 | 0.080600001 | 0.079369999 | 0.063490003 | 10 |
| MMP1 | 0.071180001 | 0.08946 | 0.079369999 | 0.063490003 | 11 |
| TIMP2 | 0.08642 | 0.07418 | 0.142900005 | 0.063490003 | 12 |
| HOXB6 | 0.096649997 | 0.102700002 | 0.079369999 | 0.063490003 | 13 |
| SPON1 | 0.067280002 | 0.07739 | 0.142900005 | 0.111100003 | 14 |
| MMP10 | 0.097649999 | 0.084119998 | 0.142900005 | 0.063490003 | 15 |
| HOXC10 | 0.1162 | 0.109800003 | 0.079369999 | 0.111100003 | 16 |
| HOXD11 | 0.089649998 | 0.093840003 | 0.142900005 | 0.111100003 | 17 |
| HOXA10 | 0.115199998 | 0.107799999 | 0.079369999 | 0.190500006 | 18 |
| HOXA9 | 0.1347 | 0.124899998 | 0.079369999 | 0.111100003 | 19 |

[symbol]

indicates data missing or illegible when filed

Additionally one may apply correction for multiple testing errors such as Benjamini-Hochberg and may apply tests for False Discovery Detection such as permutations with Bootstrap or Jack-knife algorithms.

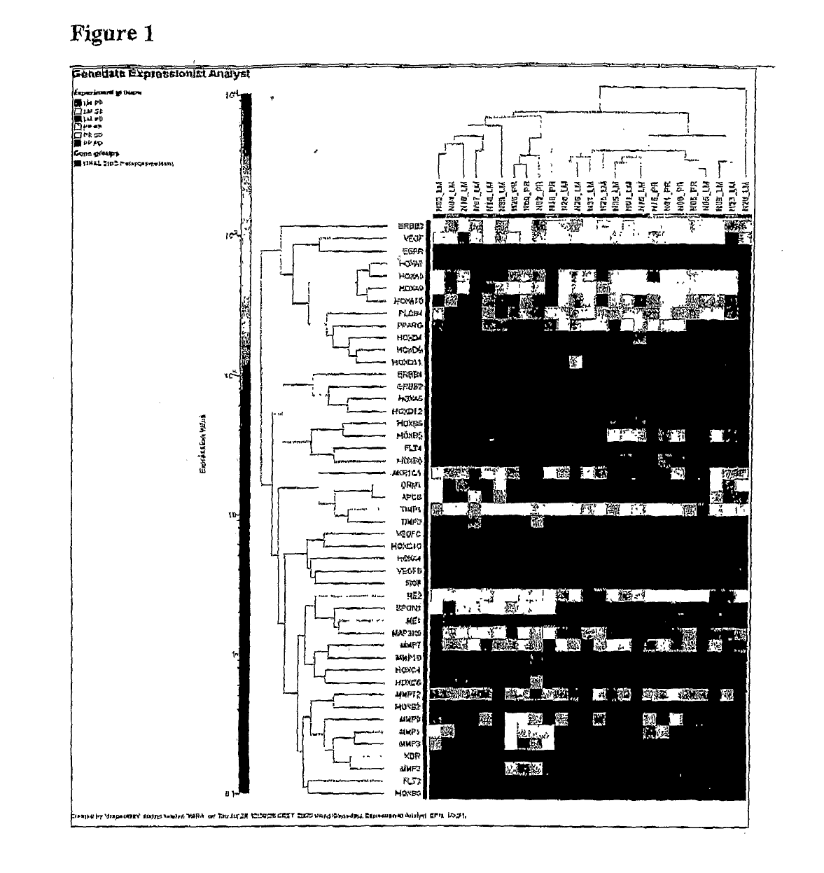

As can be seen in FIG. 1 the relative expression of the candidate genes depicted in table 1 discriminates between responding and non responding tumors. Interestingly, the liver metastasis of patient N09 and N20 cluster differently than their corresponding primary tumors. However, this is mainly due to the different expression of MMP family members. Still the primary tumors cluster in the correct group of tumors. The normalized expression of several genes is comparably low. This is due to the limited sensitivity and dynamic range of the Affymetrix platform. Subsequent experiments demonstrated that these genes can be detected by more sensitive methods (e.g., quantitative RT-PCR of RNA from FFPE tissues as shown for HOXD11; see below).

While not wishing to be bound by any theory, it was found that specific biological motifs are of predictive/prognostic value in cancer diagnostics. Of particular interest are differentiation, proliferation, invasion, apoptosis (including stress response), metabolism shift, detoxification, stroma interaction (including invasion, inflammation, acute phase marker, reorganization of ECM). By way of illustration and not by limitation these motifs are represented as follows: differentiation (HOX gene family, EGFR family), proliferation (EGFR family, MMP family), invasion (MMP family, TIMP family, EGFR family), apoptosis (MAP3K5, SPON1), metabolism shift (ME family, PPAR family), detoxification (AKR1C1), stroma interaction (MMP family, TIMP family, ORM family, VEGFR family, VEGF ligand family, SPON1, APCS).

Moreover certain signaling pathways were directly or indirectly represented by the discriminating genes depicted in table 1 (such as growth factor signaling pathways leading to MAPK-erk activation (e.g. EGFR family), pathways leading to JNK activation (e.g. MAP3K5), WNT signaling pathway (e.g. MMP7), hedgehog pathway (e.g. MMP9), APP signaling pathway (e.g. SPON1). Moreover several candidate genes are interconnected according to their biological functions (e.g. HOXA10 and PPARG are affected or part of the hormonal regulation of cellular function; PLCB4 and HOXA9 are interconnected via PKC; MMP family members affect EGFR family member function by cleaving extracellular portions; MMP family members affect growth factor ligand family members by releasing growth factors in the ECM; TIMP family members balance MMP function; APCS and SPON are involved in APP function). Another interesting aspect of the depicted candidate genes and their implication tumor response to treatment is that HOXA10 is responsible for gender specific effects within the tumor development and response to treatment.

Several genes depicted in table 1 are members of gene families and to somewhat extent coregulated. However in several cases this is due to due to non-overlapping signaling activities (e.g. MMP7 and MMP9). As these signaling activities are associated with tumor cell characteristics, the simultaneous expression of several of these genes provides improved specificity with regard to the analysis of tumor characteristics. In other cases, the expression of gene family members is normally tightly regulated excluding the simultaneous presence of multiple members of one gene family in one cell (e.g. HOX gene family members). However due to the tumor associated deregulation of the otherwise tight gene expression control, the simultaneous expression of multiple gene family members is indicative of tumor cell specific activities. Yet in other cases the simultaneous expression of more than one family member above a certain threshold level within one cell to tissue alters the biological function of the individual family members (e.g. EGFR family members; presence of EGFR alone correlates with worse outcome). It is one embodiment of this invention, that the simultaneous analysis of multiple members provides improved specificity, enables enhanced sensitivity and/or gives additional information. In summary the analysis of gene family members improves the robustness of the diagnostic methods provided within this invention.

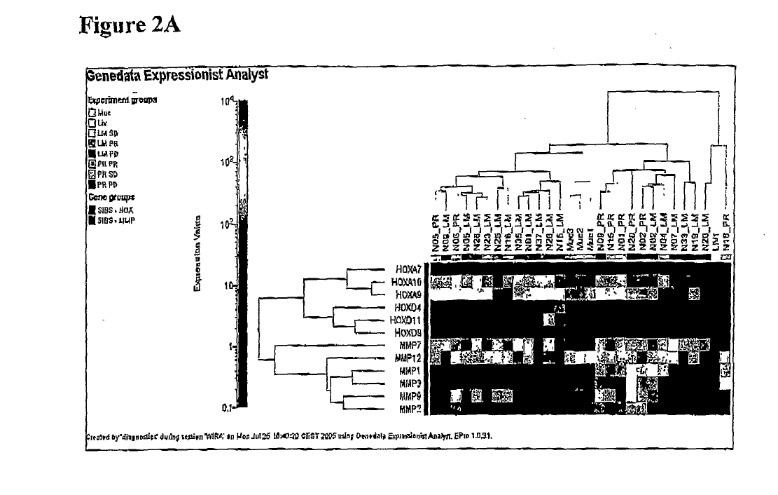

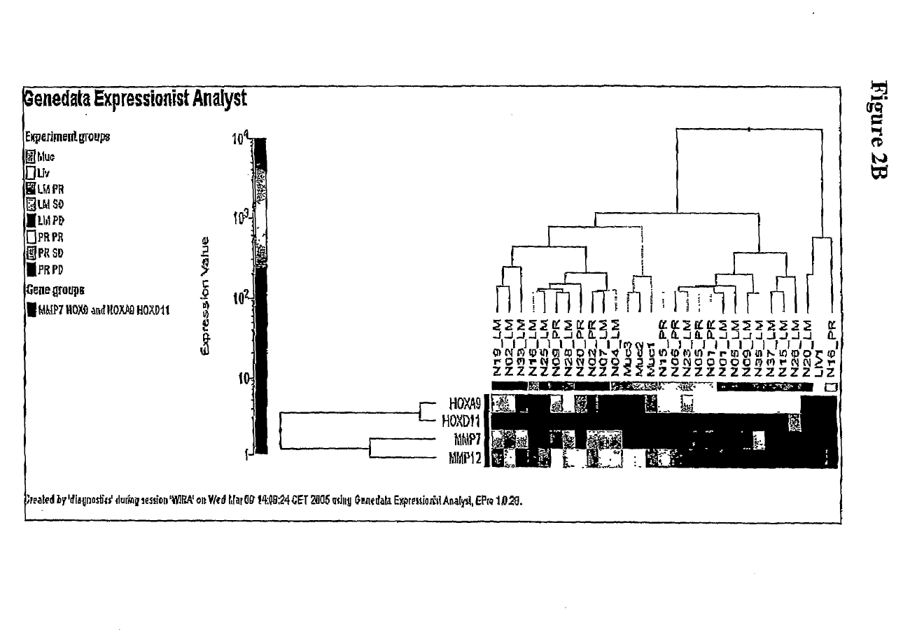

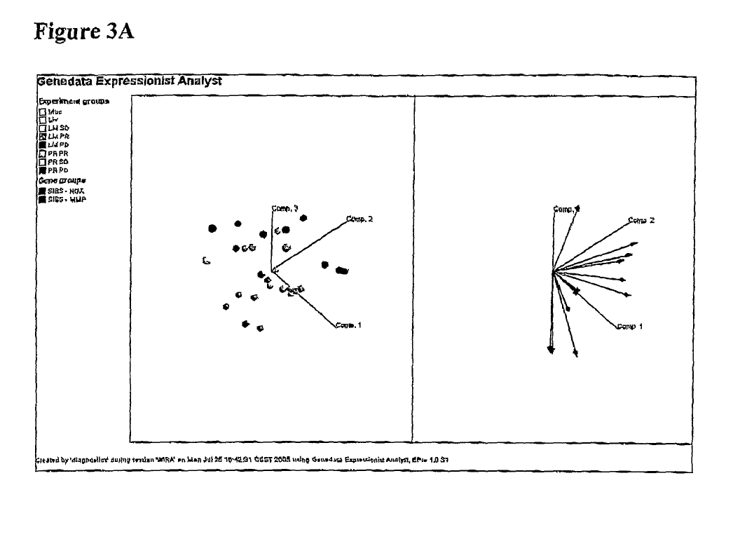

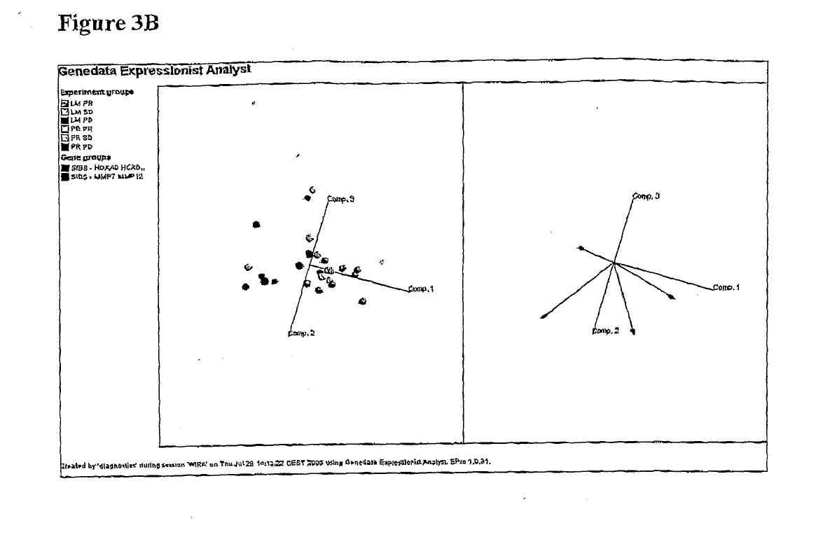

While not wishing to be bound by any theory, we have found that the analysis of more than one candidate gene exhibiting a similar expression signature across the different tumors and having related biological function as being part of one gene family improved the robustness of the prediction and prognosis. This allowed the identification of surprisingly very limited numbers of candidate genes being capable of predicting clinical outcome. As the respective candidate genes were “siblings” of one gene family (e.g. HOXA9 and HOXD11) we named the usage of the combined analysis of expression signatures “SIBS” analysis (“Smallest Informative Biological Signature”). Examples of such SIBS are presented within this invention (see e.g. FIG. 2 and FIG. 3). SIBS are meant to be representatives of defined biological motifs or activities. For example, we have found the HOX gene family to be involved in the shift between differentiation and proliferation. The degree of differentiation influences the proliferation activity of tumor cells and thereby affect the anti tumor effect of anti-proliferative substances. As another example, we have found the MMP gene family to be predictive for tumor response to treatment. Individual MMP family members are involved in tissue remodeling, migration, metastasis, growth factor receptor shedding and growth factor release from extracellular reservoirs. Both gene families have a major influence on cellular behaviour. Hox genes regulate multiple genes and central biological functions. MMP genes are regulated by multiple signaling activities (e.g. Wnt signaling, hedgehog signalling) and are of importance for cellular behaviour in the context of cell migration/metastasis, but also accessibility for anti-tumor drugs. Surprisingly the combined analysis of just these two motifs by doing SIBS analysis was sufficient for prediction of clinical outcome. As can be seen the individual members of the gene families have non-overlapping expression. We have found that the analysis of more than one gene family members (e.g. MMP J, MMP3, MMP7 and MMP12 or HOXA9, HOXA10, HOXD4, HOXD9 and HOXD11) and paired analysis of co-regulated family members improves the usefulness of the individual markers. This is depicted in FIG. 1 the combined analysis of multiple members of HOX and MMP gene family members was superior to the analysis of just one gene family or singular genes and enabled the discrimination of the tumor response to anti tumor treatment.

As can be seen in FIG. 2A the relative expression of multiple members of each gene family is similar but not identical. The combined analysis of multiple members therefore provides additional information (e.g. compare expression of HOXD4 and HOXD11). Moreover the combined SIBS analysis improves the robustness and specificity of the test. The MMP and HOX gene families are inversely regulated.

As can be seen in FIG. 2B the number of candidate genes can be reduced substantially while Still providing a very similar result, demonstrating the robustness of the SIBS analysis.

As can be seen in FIG. 3A the analysis of just two gene families is sufficient to discriminate the response of the tumors to treatment.

As can be seen in FIG. 3B the analysis of two genes of two inversely related gene families (reflecting to some extent the opposite relationship of two distinct biological motifs, i.e. differentiation vs. proliferation) is sufficient to discriminate the response of the tumors to treatment.

### EXAMPLE 2

### Expression Analysis of Primary and Metastatic Tumor Tissue by Analysis of Paraffin-Embedded Tumor Tissue

### SUMMARY

Paraffin embedded, Formalin-fixed tissues of surgical resectates of patient as described in Example 1 were analyzed and neoplastic disease marker level values were determined by qRT-PCR techniques and correlated with patient survival.

### Expression Profiling Utilizing Quantitative Kinetic RT-PCR

RNA was isolated from paraffin-embedded, formalin-fixed tissues (=FFPE tissues). Those skilled in the art are able to perform RNA extraction procedures. For example, total RNA from a 5 to 10 μm curl of FFPE tumor tissue can be extracted using the High Pure RNA Paraffin Kit (Roche, Basel, Switzerland), quantified by the Ribogreen RNA Quantitation Assay (Molecular Probes, Eugene, Oreg.) and qualified by real-time fluorescence RT-PCR of a fragment of RPL37A. In general 0.5 to 2 ng RNA of each qualified RNA extraction was assayed by qRT-PCR as described below. For a detailed analysis of gene expression by quantitative PCR methods, one will utilize primers flanking the genomic region of interest and a fluorescent labeled probe hybridizing in-between. Using the PRISM 7700 or 7900 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, one can perform such a expression measurement. Amplification of the probe-specific product causes cleavage of the probe, generating an increase in reporter fluorescence. Primers and probes were selected using the Primer Express software and localized mostly across exon/intron borders and large intervening non-transcriped sequences (> 800 bp) to guarantee RNA-specificity or with in the 3′ region of the coding sequence or in the 3′ untranslated region. Primer design and selection of an appropriate target region is well known to those with skills in the art. Predefined primer and probes for the genes listed in Table 1 can also be obtained from suppliers e.g. PE Applied Biosystems. All primer pairs were checked for specificity by conventional PCR reactions and gel electrophoresis. To standardize the amount of sample RNA, GAPDH, RPL37A, RPL9 and CD63 were selected as references, since they were not differentially regulated in the samples analyzed. To perform such an expression analysis of genes within a biological samples the respective primer/probes are prepared by mixing 25 μl of the 100 μM stock solution “Upper Primer”, 25 μl of the 100 μM stock solution “Lower Primer” with 12.5 μl of the 100 μM stock solution TaqMan-probe (FAM/Tamra) and adjusted to 500 μl with aqua dest (Primer/probe-mix). For each reaction 1.25 μl cDNA of the patient samples were mixed with 8.75 μl nuclease-free water and added to one well of a 96 Well-Optical Reaction Plate (Applied Biosystems Part No. 4306737). 1.5 μl of the Primer/Probe-mix described above, 12.5 μl Taq Man Universal-PCR-mix (2×) (Applied Biosystems Part No. 4318157) and 1 μl Water are then added. The 96 well plates are closed with 8 Caps/Strips (Applied Biosystems Part Number 4323032) and centrifuged for 3 minutes. Measurements of the PCR reaction are done according to the instructions of the manufacturer with a TaqMan 7700 from Applied Biosystems (No. 20114) under appropriate conditions (2 min. 50° C., 10 rain. 95° C., 0.15 mm. 95° C., 1 min. 60° C.; 40 cycles). Prior to the measurement of so far unclassified biological samples control experiments will e.g. cell lines, healthy control samples, samples of defined therapy response could be used for standardization of the experimental conditions.

TaqMan validation experiments were performed showing that the efficiencies of the target and the control amplifications are approximately equal which is a prerequisite for the relative quantification of gene expression by the comparative ΔΔCT method, known to those with skills in the art. Herefor the SoftwareSDS 2.0 from Applied Biosystems can be used according to the respective instructions. CT-values are then further analyzed with appropriate software (Microsoft Excel™) of statistical software packages (SAS). As well as the technology described above, provided by Perkin Elmer, one may use other technique implementations like Lightcycler™ from Roche Inc. or iCycler from Stratagene Inc. capable of real time detection of an RT-PCR reaction.

The transfer of candidate gene analysis from fresh tissue expression profiling as described in example 1 to the fixed tissue expression profiling described in example 2 has to cope with major technical differences between the test systems. These demands for the marker transfer and validation included: fresh tissue vs. fixed tissue, metastasis vs. primary tumor, microdissected material vs. whole tissue specimen, two step linear amplification vs. two step qRT-PCR, probe design against exon boundaries within the target mRNA vs. 3′ probes. Therefore the primary gene selection leading to the genes depicted in table 1 had also to refer to these technical aspects for the appropriate gene selection.

Moreover, according to the fact, that the relative expression of tumor specific candidate gene when normalized to housekeeping genes is influenced by the total amount of tumor cells within the original preparation, the tumor content of the individual FFPE tissues was estimated (table 4):

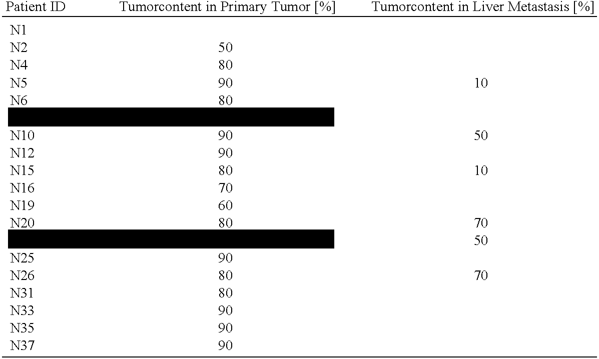

As can be seen in table 4 the analysis of two tumors (N9 and N23) is expected to be critical, as the tumor content is clearly below 30%. This is of particular interest, as the original finding of the candidate genes was done in microdissected tumor cells, which therefore contain almost exclusively tumor cells (as mentioned above).

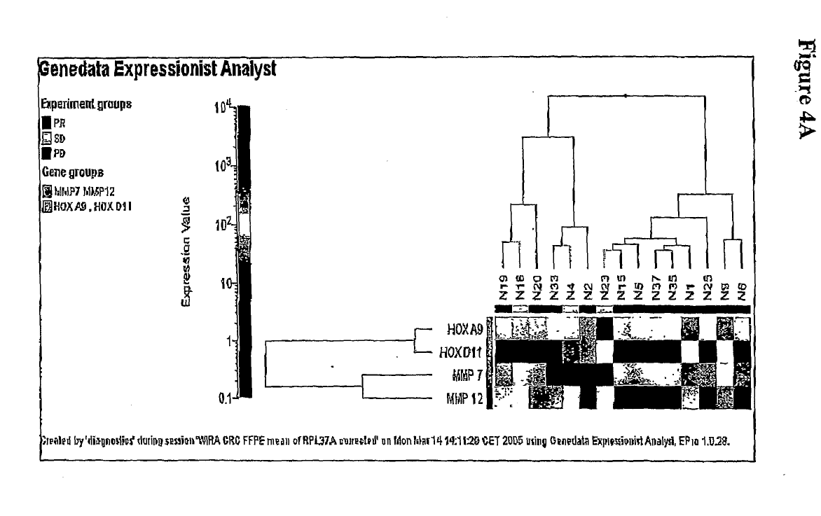

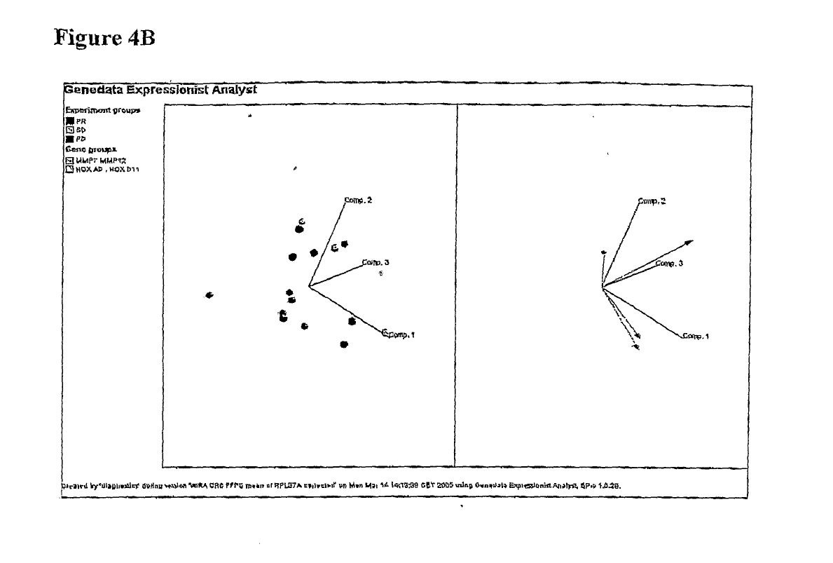

As can be seen in FIG. 4A the SIBS analysis of two genes from two different gene families resulted in a very comparable result as depicted in FIG. 2B. N9 and N23 cluster in the false groups most probably due to the low tumor content of the FFPE tissue as depicted in table 4.

As can be seen in FIG. 4A the SIBS analysis of two genes from two different gene families resulted in a very comparable result as depicted in FIG. 3B. N9 and N23 cluster in the false groups most probably due to the low tumor content of the FFPE tissue as depicted in table 4.

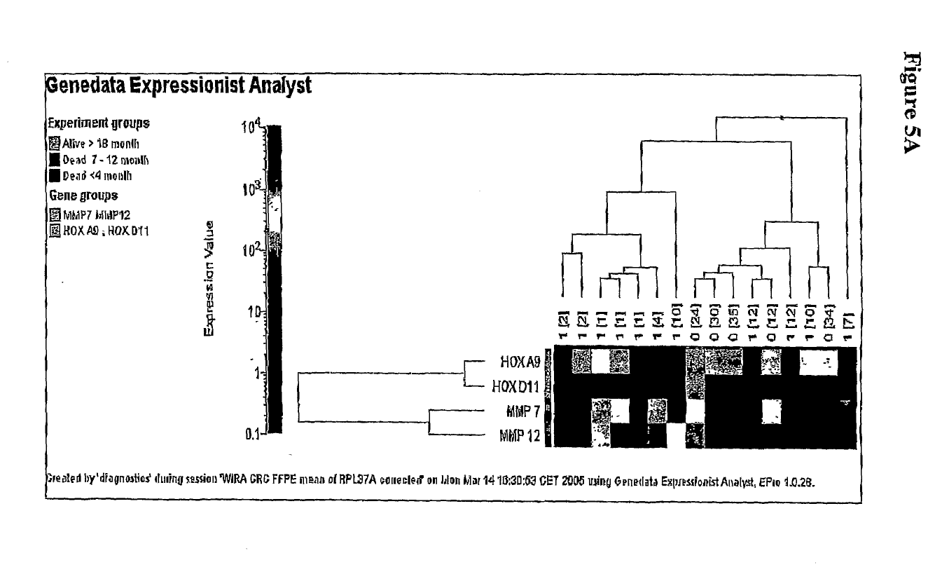

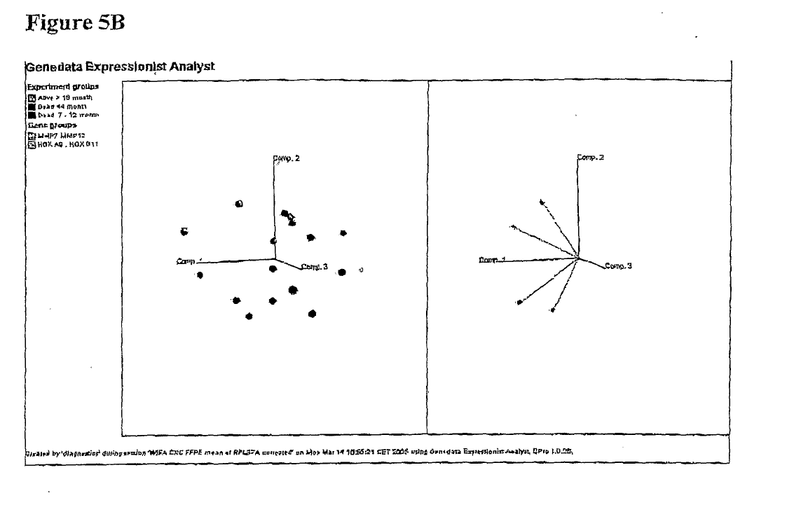

As can be seen in FIG. 5A the SIBS analysis of two genes from two different gene families resulted can distinguish between patients having a comparably good or worse prognosis due to unresponsiveness of the tumor to anti-cancer treatment. N9 and N23 cluster in the false groups most probably due to the low tumor content of the FFPE tissue as depicted in table 4.

As can be seen in FIG. 5B the SIBS analysis of two genes from two different gene families resulted can distinguish between patients having a comparably good or worse prognosis due to unresponsiveness of the tumor to anti-cancer treatment. N9 and N23 are displayed in the false groups most probably due to the low tumor content of the FFPE tissue as depicted in table A.

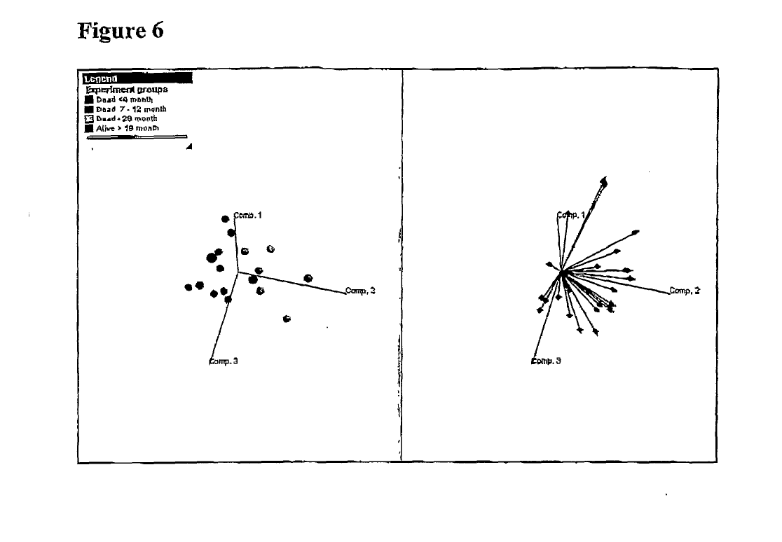

As can be seen in FIG. 6 the analysis solely the HOX gene family can distinguish between patients having a comparably good or worse prognosis due to unresponsiveness of the tumor to anti-cancer treatment. This demonstrates the biological importance of the HOX gene function with regard to prognosis and response to treatment of cancer patients.

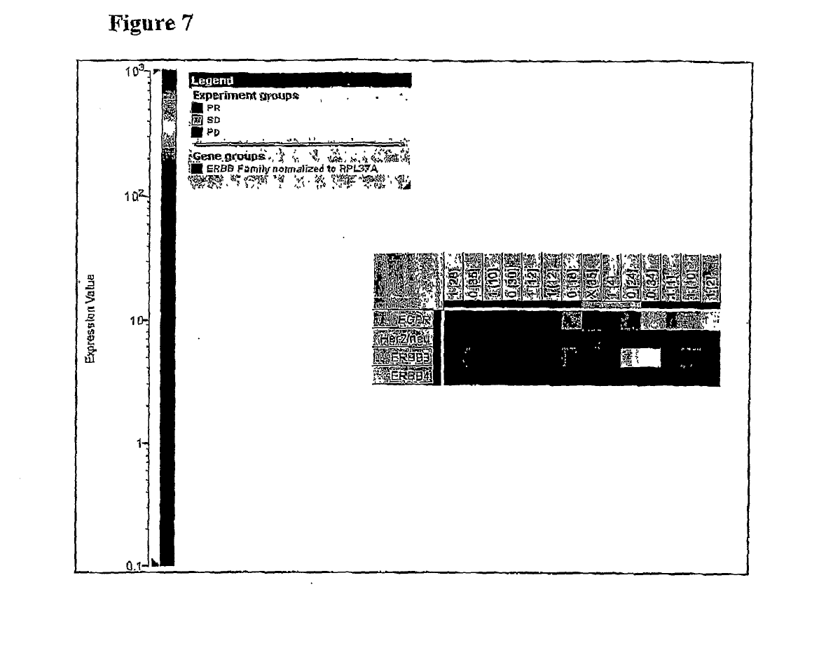

As depicted in FIG. 7, expression of EGFR family members correlates with clinical response of liver metastasis of CRC patients being treated with 5′FU based regimen as determined by CT determinations of the metastatic lesions. Clinical Response is denoted as “Partial Response” (=PR or green color bar on top), “Stable Disease” (=SD or orange color bar on top) and “Progressive Disease” (=PD or dark red color bar on top). Survival is depicted for each patient above each column (survival=0 or death=1 followed by month of survival in brackets [× month]). Clearly overexpression of at least one ERB family member is evident in the bad prognosis group, i.e. the non responding SD and PD patient cohort. Particularly high expression of EGFR in the primary tumor correlates with non-favorable response to anti-tumor treatment. This was further demonstrated by doing multiple statistical tests as depicted in Table 4 (independent of normalization method).

Elevated expression of EGFR in the bad prognosis patient cohort is of critical importance for therapeutic strategies targeted anti EGF receptor family members (like e.g. Iressa®, Erbitux® or Herceptin®), which are unexpectedly in particular useful in patients with low levels of serum EGFr. In addition, according to the data depicted in FIG. 7, the organization of the ERB family member network is of pivotal importance for the clinical outcome. Colorectal tumors expressing high levels of EGFR and simultaneously low levels of Her-2/neu do have a significantly shorter overall survival, than patients with high EGFR and Her-2/neu levels. This seems to reflect very different biological impacts of hetero- or homodimerized ERB receptors on tumorigenesis and clinical outcome of anti cancer therapies. Putatively, the composition of the ERB network influences inter alias proliferation rate thereby being of major importance for anti proliferative chemotherapeutic agents such as 5′ FU based regimens. This would explain in part the surprising finding, that Her-2/neu positive CRC tumors do have a better prognosis than Her-2/neu negative tumors.

### EXAMPLE 3

### Statistical Relevance of Candidate Genes Differentially Expressed in Cancers for Overall Survival Discrimination

While as those algorithms described can be implemented in a certain kernel to classify samples according to their specific gene expression into two classes another approach can be taken to predict class membership by implementation of a k-NN classification. The method of k-Nearest Neighbors (k-NN), proposed by T. M. Cover and P. E. Hart, an important approach to nonparametric classification, is quite easy and efficient. Partly because of its perfect mathematical theory, NN method develops into several variations. As we know, if we have infinitely many sample points, then the density estimates converge to the actual density function. The classifier becomes the Bayesian classifier if the large-scale sample is provided. But in practice, given a small sample, the Bayesian classifier usually fails in the estimation of the Bayes error especially in a high-dimensional space, which is called the disaster of dimension. Therefore, the method of k-NN has a great pity that the sample space must be large enough.

In k-nearest-neighbor classification, the training data set is used to classify each member of a “target” data set. The structure of the data is that there is a classification (categorical) variable of interest (e.g. “long-term survivors” (sample group 2) or “short-term survivors” (sample group 1)), and a number of additional predictor variables (gene expression values). Generally speaking, the algorithm is as follows:

1. For each sample in the data set to be classified, locate the k nearest neighbors of the training data set. A Euclidean distance measure or a correlation analysis can be used to calculate how close each member of the training set is to the target sample that is being examined.
2. Examine the k nearest neighbors—which classification do most of them belong to
3. Assign this category to the sample being examined.
4. Repeat this procedure steps 1 to 3 for the remaining samples in the target set.

Of course the computing time goes up as k goes up, but the advantage is that higher values of k provide smoothing that reduces vulnerability to noise in the training data. In practical applications, typically, k is in units or tens rather than in hundreds or thousands. In this disclosure we have used a k=3.

The “nearest neighbors” are determined if given the considered the vector and the distance measurement. Given a training set of expression values for a certain number of samples T={(x1, y1), (x2, y2), . . . , (xm, ym)}, to determine the class of the input vector x.

The most special case is the k-NN method, while k=1, which just searches the one nearest neighbor:

j=argmin//x−xi//

then, (x, yj) is the solution.

For estimation on the error rate of this classification the following considerations could be made:

A training set T={(x1, y1), (x2, y2), . . . , (xm, ym)} is called (k, d %)-stable if the error rate of k-NN method is d %, where d % is the empirical error rate from independent experiments. If the clustering of data are quite distinct (the class distance is the crucial standard of classification), then the k must be small. The key idea is we prefer the least k in the case that d % is bigger the threshold value.

The k-NN method gathers the nearest k neighbors and let them vote—the class of most neighbors wins. Theoretically, the more neighbors we consider, the smaller error rate it takes place. The general case is a little more complex. But by imagination, it is true to be the more k the lower upper bound asymptotic to PBayes(e) if N is fixed.

One can use such algorithm to classify and cross validate a given cohort of samples based on the genes presented by this invention in Table 1. Most preferably the classification shall be performed based on the expression levels of the genes presented in Table 1 but may also combined with clinicopathological data as fare a they are measured in a continous manner (e.g. immune histo chemistry data, scoring date such as TNM status or biochemical properties of such tumor tissue.

With k=3 and > 100 iteration one can get classifications as depicted below for a cross-validation experiment with the two classes “long-term survivors” (sample group 2) or “short-term survivors”.

The misclassification of some samples or not classifiable samples may be due to low tumor amount in specimen.

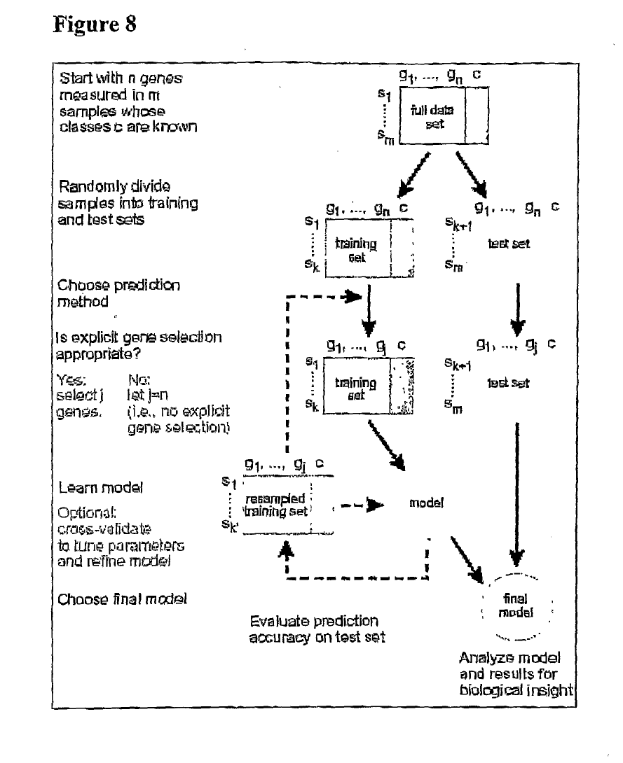

The process of model generation and cross-validation of predictive gene sets may follow the path outlined in FIG. 8 wherein a given cohort of samples is subdivided into two sets a so called training and a test set. Based on such training set genes can be picked and a preliminary model can be evaluated, further such model can be validated with the sample taken from the test set cohort. These two independent classifications of samples will lead to a final model (e.g. KNN algorithm and matrix) which can be further applied to new independent tumor samples.

In order to get the most accurate prognosis/prediction for overall survival of cancer patients based on the expression levels of genes listed in Table 1. One can implement a step wise classification model (e.g. decision tree) identifying first those individuals (tumor tissues) with the highest affinity (e.g. by k-NN classification) to the class of long term survivors tumors (good prognosis group, alive>50 month). If an so far unclassified tumor sample did not belong to this class on may perform a second classification step for this sample using the expression levels of the genes from Table 1 and some of the established clinicopathological parameters such as TNM classification. Nevertheless a classification by the genes listed in Table 1 is sufficient to identify patients not being at risk for early death or those who should receive additional treatment (e.g. Avastin, Iressa, Sorafenib, SU 11248) as being at high risk of early death (within first 27 month).

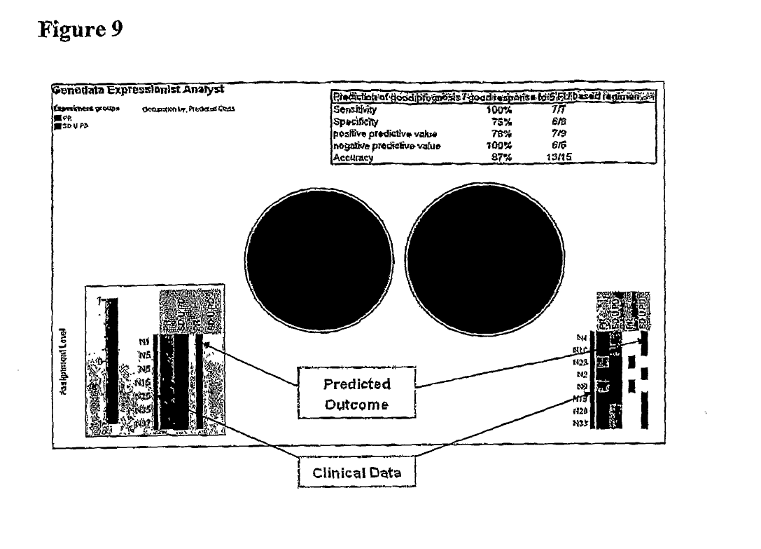

As can be seen in FIG. 9 classification based on the expression of HOXA9, HOXD11, MMP7 and MMP12 as determined by qRT-PCR and after normalization to one housekeeping gene (RPL37A), all responders can be classified correctly as having a benefit from the regimen, N9 and N23 are misclassified most probably due to the low tumor content of the FFPE tissue as depicted in table 4.

### EXAMPLE 4

In view of the small data set a linear regression model with a priori undefined and unrestricted polynomial kernels has been applied. In its basic variant this approach leads to the same combinatorial explosion as other data mining strategies. To overcome this pitfall a special strategy has been applied to select in an iterative approach optimised polynomial terms avoiding combinatorial explosion. It could be shown, that this approach leads to a stable model structure for the given data set with the functional form

Y=a+Σbᵢfᵢ(x₁, . . . , x₄),i=1 . . . 3

with multinomial function fᵢ depending on the logarithmic expression rates xᵢ of the four target genes. The parameters a and b₁ . . . b₃ have been calculated by linear regression with respect to the tumour response classification (PR=: 1, SD & PD=: 2) on the training set. The validation of the identified model has been performed using crossvalidation with splitting in training and test data set (70-75% training, 30-25% test data). In each bootstrap run the performance has been checked on the test set.

To classify the patients a cutoff value Y_{c} has to be defined, such that for all patients with

a+Σbᵢfᵢ(x₁, . . . , x₄)<Y_{c}

the classification to PR and for all patients with

a+Σbᵢfᵢ(x₁, . . . , x₄)>Y_{c}

the classification as SD or PD is done. Y_{c} has to be chosen such that the probability of misclassification will be minimised.

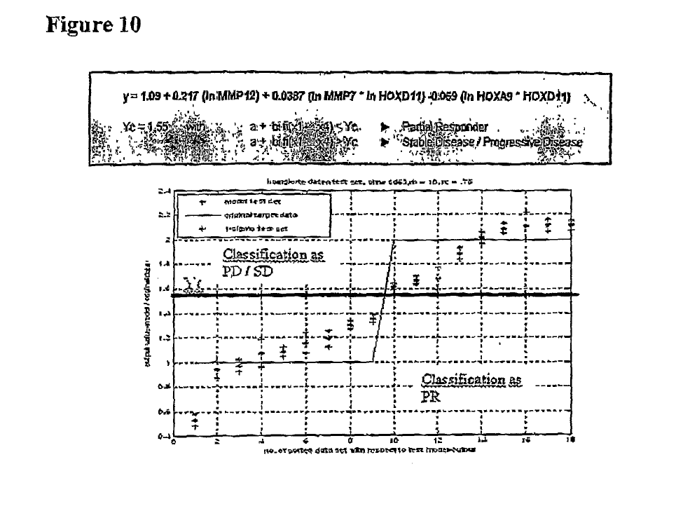

The blue stars in FIG. 10 represent the means for the model outputs for each patient (in test set), whereas the blue crosses represent the 1-sigma standard deviation of the crossvalidation—model outputs (in test set). The patients in FIG. 9 are sorted according to their mean model output. The red line depicts the respective tumour response outcomes. FIG. 9 shows that the classification of the tumour response outcomes can be predicted without misclassification in the mean of 1000 crossvalidation runs. The probability of 1 misclassification is less than 5%.

Surprisingly the algorithm allows a prediction of the misclassification probability according to the convergence properties of the iterative model identification procedure without knowledge of the true outcomes (FIG. 10).

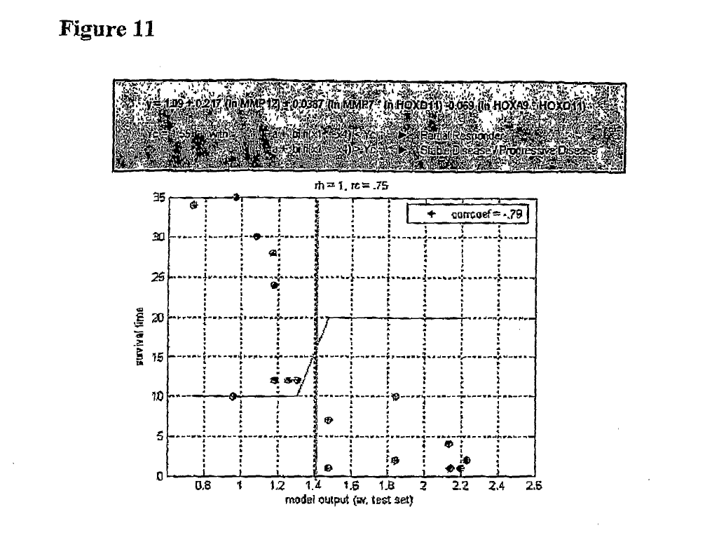

FIG. 11 depicts that the model output correlates reasonably well with the survival time (r=−0.79), although the model has been identified on tumour response classification only. Inside each tumour response classes, no information about survival times have been available to the model during the identification procedure. Therefore it is surprising that the model output correlates with the model output significantly better than expected. This result may be interpreted as follows:
the selected genes are indeed representative for the clinical outcome
the model identification procedure leads to a model structure which maps biological issues properly although the number of the available data sets is relatively small.

The data provided by SIBS analysis allow to model predictive the clinical outcome of the colon cancer therapy, tested on the basis of the available data set. The model allows to predict the survival times without retrofitting.

### REFERENCES

### Patents Cited

U.S. Pat. No. 4,683,202
U.S. Pat. No. 5,593,839
U.S. Pat. No. 5,578,832
U.S. Pat. No. 5,556,752
U.S. Pat. No. 5,631,734
U.S. Pat. No. 5,599,695
U.S. Pat. No. 4,683,195
U.S. Pat. No. 6,203,987
WO 97/29212
WO 97/27317
WO 95/22058
WO 97/02357
WO 94/13804
WO 97/14028
EP 0 785 280
EP 0 799 897
EP 0 728 520
EP 0 721 016

### OTHER REFERENCES CITED

Publications cited: WHO. International Classification of Diseases, 10^{th} edition (ICD-10). WHO
Sabin, L. H., Wittekind, C. (eds): TNM Classification of Malignant Tumors. Wiley, New York, 1997
Sorlie et al. Proc Natl Acad Sci USA. 2001 Sep. 11; 98(19): 10869-74 (3);
van t Veer et al., Nature. 2002 Jan. 31; 415(6871):530-6. (4).
Perez, E. A.: Current Managment of Metastatic Cancer. Semin. Oncol., 1999; 26 (Suppl.12): 1-10
Sambrook et al., MOLECULAR CLONING: A LABORATORY MANUAL, 2d ed., 1989
Ausubel et al., CURRENT PROTOCOLS IN MOLECULAR BIOLOGY, John Wiley & Sons, New York, N.Y., 1989.
Tedder, T. F. et al., Proc. Natl. Acad. Sci. U.S.A. 85:208-212, 1988
Hedrick, S. M. et al., Nature 308:149-153, 1984
Bonner et al., J. Mol. Biol. 81, 123 1973
Bolton and McCarthy, Proc. Natl. Acad. Sci. U.S.A. 48, 1390 1962
Hampton et al., SEROLOGICAL METHODS: A LABORATORY MANUAL, APS Press, St. Paul, Minn., 1990
Kohler et al., Nature 256,495-497, 1985
Takeda et al., Nature 314, 452-454, 1985
Burton, Proc. Natl. Acad. Sci. 88, 11120-11123, 1991
Thirion et al., Eur. J. Cancer Prev. 5, 507-11, 1996
Coloma & Morrison, Nat. Biotechnol. 15, 159-63, 1997
Mallender & Voss, L Biol. Chem. Xno9, 199-206, 1994
Verhaar et al., Int. J. Cancer 61,497-501, 1995
Orlandl et al., Proc, Natl. Acad. Sci. 86, 3833-3837, 1989
Faneyte et al., Br J Cancer, 88:406-412, 2003.
Perou et al. Nature, 406:747-752. 2000.
Sorlie et al. Proc Natl Acad Sci USA, 100:8418-8423,
Pusztai et al., Clin Cancer Res, 9:2406-2415, 2003.
Ahr et al., J. Pathol, 195:312-320, 2001.
Martin et al. Cancer Res., 60; 2232-2238, 2000.
van de Rijn et al., Am J Pathol, 161:1991-1996, 2002.
Huang et al. Lancet, 361:1590-1596, 2003.
West et al, Proc Natl Acad Sci USA, 98:11462-11467, 2001
van de Vijver et al., N Engl J. Med. 347; 1999-2009, 2002.
Sotiriou et al., Cancer Res., 4:R3, Epub 2002 Mar. 20.
Chang et al. Lancet, 362:362-369, 2003.
Korn et al, Br J Cancer, 86:1093-1096, 2002.
Adachi et al. Gut, 45,252-8, 1999
Adachi, et al. Int J Cancer, 95,290-4, 2001
An et al. Clin Exp Metastasis, 15, 184-95, 1997
Aparicio et al. Carcinogenesis, 20, 1445-53, 1999.
Balaz et al. Ann Surg, 235,519-27, 2002
Bendardaf et al. Oncology, 65, 337-46, 2003.
Bergers et al., Nat Cell Biol, 2, 737-44, 2000.
Boulay et al., Cancer Res, 61, 2189-93, 2001
Chan et al. Int J Colorectal Dis, 16, 133-40, 2001.
Coussens et al., Science, 295, 2387-92, 2002.
Crabbe et al., FEBS Lett, 345, 14-6, 1994.
Dhauasekaran et al., Nature, 412, 822-6, 2003.
Dong et al., Cell. 88, 801-10, 1997.
Fabra al., Differentiation, 52,101-10, 1992.
Giannelli al. Science, 277, 225-8, 1997.
Grau al., Clin Chem, 51, 93-101, 2005.
Hasegawa al., Int J Cancer, 76, 812-6, 1998.
Horiuchi al., J Pathol, 200,568-76, 2003.
Imai al., J Biol Chem, 270,6691-7, 1995.
Itoh, al. Cancer Res, 58,1048-51, 1998.
LaTulippe al., Cancer Res, 62, 4499-506, 2002.
Laurent al., J Am Coll Surg, 198, 884-91, 2004.
Leeman al., J Pathol, 201, 528-34, 2003.
Liabakk al., Cancer Res, 56, 190-6, 1996.
Lochter al., J Cell Biol, 139,1861-72, 1997.
Lozonschi al., Cancer Res, 59, 1252-8, 1999
Masaki al., Br J Cancer, 84,1317-21, 2001.
Masuda al., Dis Colon Rectum, 42, 393-7, 1999.
Matsuyama al., J Surg Oncol, 80,105-10, 2002.
McDonnell al., Mol Carcinog, 4, 527-33, 1991.
Murray al., Nat Med, 2, 461-2, 1996,
Newell al., Mol Carcinog, 10,199-206, 1994.
Parsons, al., Br J Cancer, 78, 1495-502, 1998.
Ramos-DeSimone al., J Biol Chem, 274,13066-76, 1999.
Roeb al., Cancer, 92,2680-91, 2001.
Roeb al., Int J Colorectal Dis, 19,518-24, 2004.
SaueraL, N Engl J Med, 351, 1731-40, 2004.
Shah al., In Vivo, 8,321-6, 1994.
Shiozawa al., Mod Pathol, 13, 925-33, 2000.
Sunami al., Oncologist, 5, 108-14, 2000.
Visse al, Circ Res, 92, 827-39, 2003.
Wagenaar-Miller al., Cancer Metastasis Rev, 23,119-35, 2004.
Wilson al., Int J Biochem Cell Biol, 28, 123-36, 1996.
Yang al. Cancer, 91, 1277-83, 2001.
Zeng al., J Clin Oncol, 14, 3133-40, 1996.
Zeng al., Br J Cancer, 78, 349-53, 1998.
Zeng al., Carcinogenesis, 20, 749-55, 1999.
Zeng al. Clin Cancer Res, 8, 144-8, 2002.
Nunes F. D. et al., Pesqui Odontol Bras. 17(1):94-8. Epub 2003 Aug. 5.
Cillo C., Invasion Metastasis.; 14(1-6):38-49, 1994-95.
De Vita et al., Eur J Cancer 29A(6):887-93, 1993.
Zakany et al. Nature 401: 761-762, 1999.
Greer et al. Nature 403: 661-665, 2000.
Scott (Letter) Cell 71: 551-553, 1992.
Folkman, J., Nature Med 1: 27-31, 1995.

1. A method for predicting therapeutic success of a given mode of treatment in a subject having cancer, comprising:
(i) determining the pattern of expression levels of at least one marker genes, comprised in selected from the group of marker genes listed in Table 1;
(ii) comparing the pattern of expression levels determined in (i) with one or several reference pattern(s) of expression levels; and
(iii) predicting therapeutic success for said given mode of treatment in said subject from the outcome of the comparison in step (ii).

2. A method for adapting therapeutic regimen based on individualized risk assessment for a subject having cancer, comprising:
(i) determining the pattern of expression levels of at least one marker genes, comprised in selected from the group of marker genes listed in Table 1;
(ii) comparing the pattern of expression levels determined in (i) with one or several reference pattern(s) of expression levels; and
(iii) implementing a therapeutic regimen targeting said marker genes in said subject from the outcome of the comparison in step (ii).

3. The method of claim 1, wherein said given mode of treatment comprises a treatment selected from the group consisting of:
(i) a treatment that acts on recruitment of lymphatic vessels;
(ii) a treatment that acts on cell proliferation;
iii) a treatment that acts on cellular differentiation;
iv) a treatment that acts on cell motility;
v) a treatment that acts on cell survival;
vi) a treatment that acts on cellular metabolism;
vii) a treatment that acts on detoxification;
viii) a treatment that comprises administration of a chemotherapeutic agent; and combinations thereof.

4. The method of claim 1, wherein said given mode of treatment comprises a treatment selected from the group consisting of:
chemotherapy;
treatment with small molecule inhibitors;
an antibody based regimen (Trastuzumab, avastin);
an anti-proliferation regimen;
a pro-apoptotic regimen;
a pro-differentiation regimen;
radiation;
surgical therapy;
and combinations thereof.

5. The method of claim 1, wherein a predictive algorithm is used.

6. A method of treatment of a neoplastic disease in a subject, comprising;
i) predicting a therapeutically successful mode of treatment in a subject having cancer by the method of claim 1; and
ii) treating said neoplastic disease in said subject by said predicted therapeutically successful mode.

7. A method of selecting a therapy modality for a subject afflicted with a neoplastic disease, comprising;
i) obtaining a biological sample from said subject;
ii) predicting from said sample, by the method of claim 1, therapeutic success in a subject having cancer for a plurality of individual modes of treatment; and
iii) selecting a mode of treatment which is predicted to be successful in step (ii).

8. The method of any of claim 1, wherein the expression level is determined by a method selected from the group consisting of:
i) a hybridization based method;
ii) a hybridization based method utilizing arrayed probes;
iii) a hybridization based method utilizing individually labeled probes;
iv) real time PCR;
v) assessing the expression of polypeptides, proteins or derivatives thereof;
vi) assessing the amount of polypeptides, proteins or derivatives thereof;
and combinations thereof.

9. A kit comprising;
at least one primer pair, wherein each primer pair is specific to a marker gene listed in Table 1;
and at least one probe, each probe having a sequence complementary to the sequence of a marker gene listed in Table 1.

10. A kit comprising at least one individually labeled probe, each probe having a sequence complementary to the sequence of a marker gene listed in Table 1.

11. A kit comprising at least one arrayed probe, each probe having a sequence complementary to the sequence of a marker gene listed in Table 1.

12. The method of claim 1, wherein the at least one marker gene comprises a set of marker genes selected from the group consisting of: at least 1, at least 2, at least 3, at least 4, at least 5, at least 10, at least 15, at least 20, at least 25, at least 30, at least 35 or at least 48 marker genes listed in Table 1.

13. The method of claim 2, wherein the at least one marker gene comprises a set of marker genes selected from the group consisting of: at least 1, at least 2, at least 3, at least 4, at least 5, at least 10, at least 15, at least 20, at least 25, at least 30, at least 35 or at least 48 marker genes listed in Table 1.

14. The kit of claim 10, wherein the at least one individually labeled probe comprises a set of individually labeled probes selected from the group consisting of: at least 1, at least 2, at least 3, at least 4, at least 5, at least 10, at least 15, at least 20, at least 25, at least 30, at least 35 or at least 48 individually labeled probes;
wherein each probe of the set has a sequence complementary to the sequence of a marker gene listed in Table 1.

15. The kit of claim 11, wherein the at least one arrayed probe comprises a set of arrayed probes selected from the group consisting of: at least 1, at least 2, at least 3, at least 4, at least 5, at least 10, at least 15, at least 20, at least 25, at least 30, at least 35 or at least 48 arrayed probes;
wherein each probe of the set has a sequence complementary to the sequence of a marker gene listed in Table 1.

16. The method of claim 3, wherein said given mode of treatment comprises a treatment selected from the group consisting of:
chemotherapy;
treatment with small molecule inhibitors;
an antibody based regimen;
an anti-proliferation regimen;
a pro-apoptotic regimen;
a pro-differentiation regimen;
radiation;
surgical therapy;
and combinations thereof.

17. The method of claim 2, wherein a predictive algorithm is used.

18. The method of claim 1, wherein the expression level is determined by a method selected from the group consisting of:
i) a hybridization based method;
ii) a hybridization based method utilizing arrayed probes;
iii) a hybridization based method utilizing individually labeled probes;
iv) real time PCR;
v) assessing the expression of polypeptides, proteins or derivatives thereof;
vi) assessing the amount of polypeptides, proteins or derivatives thereof; and combinations thereof.

19. The method of claim 2, wherein the expression level is determined by a method selected from the group consisting of:
i) a hybridization based method;
ii) a hybridization based method utilizing arrayed probes;
iii) a hybridization based method utilizing individually labeled probes;
iv) real time PCR;
v) assessing the expression of polypeptides, proteins or derivatives thereof;
vi) assessing the amount of polypeptides, proteins or derivatives thereof;
and combinations thereof.
